(19) United States
(12) Patent Application Publication
(10) Pub. No.: US 2026/0218419 A1
(43) Pub. Date: Jul. 30, 2026

(54) METHOD FOR CONSTRUCTING A DNA LIBRARY, ADAPTER ELEMENT, AND KIT

(71) Applicant: Nanodigmbio (Nanjing) Biotechnology Co., LTD., Jiangsu (CN)

(72) Inventors: Yugang HU, Jiangsu (CN); Mingye ZHANG, Jiangsu (CN); Biao WANG, Jiangsu (CN); Youyou WANG, Jiangsu (CN); Qiang WU, Jiangsu (CN)

(73) Assignee: Nanodigmbio (Nanjing) Biotechnology Co., LTD., Jiangsu (CN)

(21) Appl. No.: 19/299,616

(22) Filed: Aug. 14, 2025

(30) Foreign Application Priority Data
Jan. 26, 2025 (CN) 202510124654.3

(51) Int. Cl. C40B 50/06; C12Q 1/6806; C12Q 1/6855; C12Q 1/6876

(52) U.S. Cl. CPC C40B 50/06; C12Q 1/6806; C12Q 1/6855; C12Q 1/6876

(57) ABSTRACT

Provided in the present application is a method for constructing a DNA library, an adapter element, and a Kit. The method includes performing library construction on a target sample utilizing a primer with 5′ phosphorylation modification or an adapter with 5′ phosphorylation modification, obtaining a linear amplification library with 5′ phosphorylation modification, where the linear amplification library with 5′ phosphorylation modification is a linear library suitable for an Illumina sequencing platform; or further circularizing the linear amplification library with 5′ phosphorylation modification to obtain a circularized library suitable for an MGI sequencing platform.

### CROSS-REFERENCE TO RELATED APPLICATION(S)

This application claims priority to Chinese Patent Application No. 202510124654.3 filed on Jan. 26, 2025, the disclosure of which is hereby incorporated by reference in its entirety as part of this application.

### SEQUENCE LISTING

The present application includes a sequence listing submitted electronically in XML format, which is hereby incorporated by reference in its entirety. The XML file is named 45540_Amended SequenceListing.xml, created on Feb. 3, 2026, with a file size of 722, 659 bytes. The sequence listing contains 826 sequences numbered SEQ ID NO: 1 to 826, which is substantially the same as that disclosed in the Chinese patent application No. 2025101246543 and the Sequence Listing filed Aug. 1, 2025, except that the SEQ ID NO: 826 has been added. The sequence listing does not contain any new content.

### FIELD

The present disclosure relates to the field of DNA library construction, and specifically, to a method for constructing a DNA library, an adapter element, and a kit.

### BACKGROUND

With the development and maturation of second-generation DNA sequencing technology, second-generation sequencers have flourished under innovation-driven development. At present, the second-generation sequencers on market have formed a coexisting situation of two sequencing giants (Illumina Inc. and MGI Tech Co., Ltd) and a plurality of new entrants (Element Biosciences, Inc., GeneMind Biosciences Company, etc.). Since accurate medical diagnoses currently rely heavily on high-throughput second-generation sequencing, the throughput of sequencers is increasing in order to reduce sequencing costs and increase product competitiveness. The models and sequencing throughput of the latest sequencers from Illumina® sequencing platform respectively are NovaSeq™ 6000, which produces 6 Tb of data, and Novaseq™ X Plus, which produces 16 Tb of data. The models and sequencing throughput of the latest sequencers from MGI® sequencing platform respectively are MGI® DNBSEQ™-T7, which produces 6Tb of data, and DNBSEQ™-T20X2, which produces 72 Tb of data. The increase in sequencer throughput has proposed higher requirements on library index adapters, requiring them to offer advantages such as high quality and variety.

Although there are various sequencing platforms currently available on the market, these sequencing platforms may solve all loading problems using a universal library. For example, Patent CN113999893B solves the compatibility and base balance problems of Illumina® sequencing platform and MGI® sequencing platform. However, with the development of the sequencing technology and higher-throughput sequencers, the types and quality of library index adapters in the prior art still have certain limitations, such that developing various high-quality library index adapters is a key problem that needs to be solved urgently.

### SUMMARY

The present disclosure is mainly intended to provide a method for constructing a DNA library, an adapter element, and a kit, so as to solve the problem that the types and quality of library index adapters in the prior art do not meet requirements for high-throughput sequencing library construction.

In order to implement the above objective, a first aspect of the present disclosure provides a method for constructing a DNA library. The method includes: library construction is performed on a target sample utilizing a primer with 5′ phosphorylation modification or an adapter with 5′ phosphorylation modification, obtaining a linear amplification library with 5′ phosphorylation modification, where the above linear amplification library with 5′ phosphorylation modification is a linear library suitable for an Illumina® sequencing platform.

Alternatively, the linear amplification library with 5′ phosphorylation modification is further circularized to obtain a circularized library suitable for an MGI® sequencing platform.

The above primer with 5′ phosphorylation modification includes a P5 truncated amplification primer; the above adapter with 5′ phosphorylation modification includes a P5 full-length adapter and a P7 full-length adapter.

The above P5 truncated amplification primer has the following sequences: 5′-SEQ ID NO: 821-NNNNNNNNNN-SEQ ID NO: 819-3′, wherein a sequence of SEQ ID NO: 821 is AATGATACGGCGACCACCGAGATCTACAC, a sequence of SEQ ID NO: 819 is ACACTCTTTCCCTACACGAC, and N represents a P5-end index sequence; and a 5′ end of the above P5 truncated amplification primer is modified through phosphorylation.

A P7 truncated amplification primer has the following sequences: 5′-SEQ ID NO: 823-NNNNNNNNNN-SEQ ID NO: 820-3′, where a sequence of SEQ ID NO: 823 is CAAGCAGAAGACGGCATACGAGAT, a sequence of SEQ ID NO: 820 is GTGACTGGAGTTCAGACGTGT, and N represents a P7-end index sequence.

The above P5 full-length adapter has the following sequences: 5′-SEQ ID NO: 821-NNNNNNNNNN-SEQ ID NO: 817-3′, where a sequence of SEQ ID NO: 821 is AATGATACGGCGACCACCGAGATCTACAC, a sequence of SEQ ID NO: 817 is ACACTCTTTCCCTACACGACGCTCTTCCGATC*T, N represents the P5-end index sequence, and * represents thio modification; and a 5′ end of the above P5 full-length adapter is modified through phosphorylation.

The above P7 full-length adapter has the following sequences: 5′-SEQ ID NO: 818-NNNNNNNNNN-SEQ ID NO: 824-3′, wherein a sequence of SEQ ID NO: 818 is GATCGGAAGAGCACACGTCTGAACTCCAGTCAC, a sequence of SEQ ID NO: 824 is ATCTCGTATGCCGTCTTCTGCTTG, and N represents the P7-end index sequence; and a 5′ end of the above P7 full-length adapter is modified through phosphorylation.

The sequences, which are 1 bp from upstream and downstream of the above index sequence including the above P5-end index sequence or the above P7-end index sequence, have at least three edit distances.

A plurality of target samples are provided, and the above P5-end index sequences corresponding to the plurality of target samples are selected from P5-end index sequences in any one of loading combinations in Table 1; the above P7-end index sequence is selected from fixedly-matched P7-end index sequences corresponding to the above P5-end index sequences in Table 1, where the above P5-end index sequences and the above P7-end index sequences all meet the respective number of bases A, T, C, and G in the above loading combinations being ≥12.5%; and 8 indexes constitute a group of the above loading combinations, and Table 1 is as follows.

TABLE 1

| | SEQ ID | p5-end | P5-end | SEQ ID | p7-end | P7-end |
|---|---|---|---|---|---|---|
| Number | NO: | number | sequence | NO: | number | sequence |
| TY491 | 1 | p5-001 | GACCTCGGTT | 409 | p7-001 | ACCTTGTGTT |
| TY324 | 2 | p5-002 | CGGAAGCTGA | 410 | p7-002 | GGAAGTGACC |
| TY025 | 3 | p5-003 | ACGCGTTCAA | 411 | p7-003 | TATCCTCTGG |
| TY266 | 4 | p5-004 | GTTGCAAGTC | 412 | p7-004 | CTAGACCAAT |
| TY124 | 5 | p5-005 | TTCTGGATCG | 413 | p7-005 | AGCAGTACTA |
| TY197 | 6 | p5-006 | GCAGAACATT | 414 | p7-006 | AAGCCATGAG |
| TY370 | 7 | p5-007 | AATTCCTACC | 415 | p7-007 | TCTTAGCCGA |
| TY299 | 8 | p5-008 | TGAAGACCAA | 416 | p7-008 | GCGATAATAC |
| TY438 | 9 | p5-009 | CTTCTGAGTA | 417 | p7-009 | AGAGGCATGG |
| TY252 | 10 | p5-010 | AAGTGCCAGG | 418 | p7-010 | TTCTATCCTC |
| TY340 | 11 | p5-011 | GCAAGTATAC | 419 | p7-011 | TGTCTGGACT |
| TY164 | 12 | p5-012 | TGGCAATGGC | 420 | p7-012 | CAGCGGTGAA |
| TY486 | 13 | p5-013 | TTCGTTACCT | 421 | p7-013 | ACAACTACAG |
| TY344 | 14 | p5-014 | CTTGCGGTAA | 422 | p7-014 | GAGGAAGTAA |
| TY474 | 15 | p5-015 | GGAACACATT | 423 | p7-015 | ATTGTCAGCT |
| TY503 | 16 | p5-016 | ACATAGTCCT | 424 | p7-016 | TCGCCAGATC |
| TY444 | 17 | p5-017 | GAGATCTTGT | 425 | p7-017 | TCGACACAGA |
| TY169 | 18 | p5-018 | CGTGCTAACC | 426 | p7-018 | GAATCCACAG |
| TY391 | 19 | p5-019 | ATAAGGCCTC | 427 | p7-019 | CCTCTTGATT |
| TY182 | 20 | p5-020 | TCGTACGGAA | 428 | p7-020 | ATCTGGAGGT |
| TY082 | 21 | p5-021 | ATTCCACCGA | 429 | p7-021 | GGTGAGCTCA |
| TY092 | 22 | p5-022 | TACGTACTCT | 430 | p7-022 | CTCCAATTCC |
| TY381 | 23 | p5-023 | GGCCTTAGAG | 431 | p7-023 | TGACGTCAAT |
| TY481 | 24 | p5-024 | CCAAGTGAGA | 432 | p7-024 | AAGAATGGAC |
| TY513 | 25 | p5-025 | GGTTGTAACT | 433 | p7-025 | ATTCCACACA |
| TY276 | 26 | p5-026 | CTCCTCGCAA | 434 | p7-026 | TGGTTCTGAA |
| TY085 | 27 | p5-027 | TATGACTGTG | 435 | p7-027 | TACAATGCTC |
| TY200 | 28 | p5-028 | AGAACAGTCC | 436 | p7-028 | GTAGTGGTGC |
| TY103 | 29 | p5-029 | ATGCTGCGGA | 437 | p7-029 | CGATCCATTG |
| TY422 | 30 | p5-030 | GACTCACAGC | 438 | p7-030 | ACAAGACTGT |
| TY076 | 31 | p5-031 | TCTCGGTTAT | 439 | p7-031 | CGCGGTTCAT |
| TY505 | 32 | p5-032 | GGATATACCA | 440 | p7-032 | ATTCGGAACA |
| TY022 | 33 | p5-033 | CGCAGATCCA | 441 | p7-033 | ACACAAGAAC |
| TY421 | 34 | p5-034 | TAGCCTATGG | 442 | p7-034 | GTTACCAGCG |
| TY037 | 35 | p5-035 | ACTGACCATA | 443 | p7-035 | GAGTTGGCTC |
| TY349 | 36 | p5-036 | GGCTACTGCT | 444 | p7-036 | TTCGGTTAGT |
| TY177 | 37 | p5-037 | CTAGTGTACC | 445 | p7-037 | CGAGCCTCAA |
| TY242 | 38 | p5-038 | TCTTCGGAGG | 446 | p7-038 | AGCAGGCTCA |
| TY502 | 39 | p5-039 | TTGACCAGAC | 447 | p7-039 | CAGTATAGAG |
| TY188 | 40 | p5-040 | CAACTTGCAA | 448 | p7-040 | GGTTCACCGT |
| TY396 | 41 | p5-041 | GGTTGATCGA | 449 | p7-041 | TGTTCGTTCG |
| TY168 | 42 | p5-042 | ATAATGGTCG | 450 | p7-042 | ACAGTCCATT |
| TY466 | 43 | p5-043 | GACAACCATT | 451 | p7-043 | CTGTGAAGAT |
| TY017 | 44 | p5-044 | ACGGTCACAC | 452 | p7-044 | GCTATTCTGA |
| TY105 | 45 | p5-045 | CGGCCAATGT | 453 | p7-045 | GACGAGGAAT |
| TY130 | 46 | p5-046 | TGGAATTGTC | 454 | p7-046 | TTGACAGCGC |
| TY309 | 47 | p5-047 | CAACGAGTCG | 455 | p7-047 | AGCCGATGTC |
| TY056 | 48 | p5-048 | ACCTCCACAA | 456 | p7-048 | TAACACAGTG |
| TY251 | 49 | p5-049 | GGAATCTCCT | 457 | p7-049 | ACTAGCGGAC |
| TY480 | 50 | p5-050 | AAGCGAGGAA | 458 | p7-050 | TTAGCAACGT |
| TY132 | 51 | p5-051 | CATTAGATCG | 459 | p7-051 | CACAATATCG |
| TY475 | 52 | p5-052 | TTCTTCTAGC | 460 | p7-052 | ACCTGGTATA |
| TY399 | 53 | p5-053 | ATTGCTCCTA | 461 | p7-053 | ACACTTCTTC |
| TY222 | 54 | p5-054 | GCGATAATCT | 462 | p7-054 | TGGTCAGCAG |
| TY295 | 55 | p5-055 | TGTCGTAGTG | 463 | p7-055 | GTATTCCAGA |
| TY523 | 56 | p5-056 | AAGTCCGAAC | 464 | p7-056 | CGTGACTGCT |
| TY180 | 57 | p5-057 | CAAGTCACAA | 465 | p7-057 | AAGCCTCCAT |
| TY174 | 58 | p5-058 | GCCTCATGGT | 466 | p7-058 | ATCTGAATGC |
| TY429 | 59 | p5-059 | TGGCGTCTGT | 467 | p7-059 | GGTAACAGAG |
| TY265 | 60 | p5-060 | ACTCAGGACG | 468 | p7-060 | CCGACTTCCA |
| TY282 | 61 | p5-061 | TAGAGAGGTC | 469 | p7-061 | TCAGTGGAAC |
| TY482 | 62 | p5-062 | GTCTCGCTTC | 470 | p7-062 | TGTCTCTGTT |
| TY109 | 63 | p5-063 | CTTAGCTCAA | 471 | p7-063 | AATTACCGGA |
| TY277 | 64 | p5-064 | GCACAGAACT | 472 | p7-064 | GTCATAATCG |
| TY394 | 65 | p5-065 | CGTTGCATCT | 473 | p7-065 | TTAAGCTGGA |
| TY424 | 66 | p5-066 | ATGAATGCGA | 474 | p7-066 | TATGCTGCAC |
| TY112 | 67 | p5-067 | ACACTATGTG | 475 | p7-067 | CCGATAACTG |
| TY061 | 68 | p5-068 | TGCTAGAGCC | 476 | p7-068 | GGCCTGCATT |
| TY387 | 69 | p5-069 | CAGCTACCAC | 477 | p7-069 | AAGTACGTAC |
| TY118 | 70 | p5-070 | GTAGCCAATA | 478 | p7-070 | TCCTTATGCC |
| TY501 | 71 | p5-071 | GATGGATTGT | 479 | p7-071 | CTACGGCCTA |
| TY400 | 72 | p5-072 | TCCATGGAGC | 480 | p7-072 | ACCTATGAGA |
| TY291 | 73 | p5-073 | GAATAAGCCA | 481 | p7-073 | AGCCGCGTAT |
| TY011 | 74 | p5-074 | AGTATGCTGA | 482 | p7-074 | TTGTTGTCTG |
| TY013 | 75 | p5-075 | TTCTACTGTC | 483 | p7-075 | CCAGAACGGA |
| TY001 | 76 | p5-076 | CCGGAATATT | 484 | p7-076 | GGAACGATCC |
| TY060 | 77 | p5-077 | GAGCGGTAAG | 485 | p7-077 | CATCCTGCAG |
| TY303 | 78 | p5-078 | GTAGCTAGGC | 486 | p7-078 | AACGCTTAGA |
| TY035 | 79 | p5-079 | CACATTACGT | 487 | p7-079 | TTGATTGACC |
| TY134 | 80 | p5-080 | TCCTTCGTTA | 488 | p7-080 | GCCTTACGTT |
| TY055 | 81 | p5-081 | CGTGGTGCTA | 489 | p7-081 | ATATCAGTCC |
| TY048 | 82 | p5-082 | ACGACAAGAC | 490 | p7-082 | TCCGACAGGA |
| TY077 | 83 | p5-083 | GAATACCTCG | 491 | p7-083 | AAGATTGCTG |
| TY245 | 84 | p5-084 | TGGCTGGTGT | 492 | p7-084 | TATCGGTACG |
| TY258 | 85 | p5-085 | CTTCAACGAC | 493 | p7-085 | CGAGAACTAT |
| TY392 | 86 | p5-086 | TCCACGTAGG | 494 | p7-086 | GACAGAAGCG |
| TY450 | 87 | p5-087 | GTCAGGCACA | 495 | p7-087 | GCATTGCCTC |
| TY373 | 88 | p5-088 | GGATTCACTT | 496 | p7-088 | AGTACTGAAG |
| TY343 | 89 | p5-089 | AAGCTCCATA | 497 | p7-089 | ACATCGCCTA |
| TY256 | 90 | p5-090 | GTCAATATCC | 498 | p7-090 | TTCCAAGTCG |
| TY545 | 91 | p5-091 | CTATCGGCCT | 499 | p7-091 | AGGACTAATC |
| TY005 | 92 | p5-092 | GGTCAAGGTG | 500 | p7-092 | CCTGGATGGA |
| TY506 | 93 | p5-093 | CAGGTGCAGT | 501 | p7-093 | GATAGCTGAT |
| TY154 | 94 | p5-094 | TCACGACTAA | 502 | p7-094 | CGCGTTAGGT |
| TY407 | 95 | p5-095 | ATCGGTTGAA | 503 | p7-095 | TACCAGCTAT |
| TY354 | 96 | p5-096 | ACTTCATTGC | 504 | p7-096 | CTCTACGTCG |
| TY047 | 97 | p5-097 | GGCGAGACAA | 505 | p7-097 | TTATGAGGCC |
| TY041 | 98 | p5-098 | CTGCCTTGTG | 506 | p7-098 | ACCAAGCAGG |
| TY551 | 99 | p5-099 | ACCTTCAAGT | 507 | p7-099 | GTGGACAGAA |
| TY111 | 100 | p5-100 | ATAACGCGCA | 508 | p7-100 | CGACCTTCTG |
| TY075 | 101 | p5-101 | TGTGGAGTCC | 509 | p7-101 | TTACACTCGT |
| TY552 | 102 | p5-102 | GAACTTCGGA | 510 | p7-102 | TACGTTGTTC |
| TY101 | 103 | p5-103 | CATAACTTCG | 511 | p7-103 | AATTCAGGCA |
| TY224 | 104 | p5-104 | TCCTGGCATT | 512 | p7-104 | TCGATGCTAA |
| TY404 | 105 | p5-105 | CGATGCACGA | 513 | p7-105 | AATTCTCACG |
| TY098 | 106 | p5-106 | ACTATACACC | 514 | p7-106 | TCGCAGTGAC |
| TY087 | 107 | p5-107 | CGACCTGTGT | 515 | p7-107 | GGCAGAGTTA |
| TY538 | 108 | p5-108 | GACAGTTGTA | 516 | p7-108 | TTCTTAACGG |
| TY131 | 109 | p5-109 | TTGGCTGCCT | 517 | p7-109 | CAATGCCAAT |
| TY031 | 110 | p5-110 | CACAACCGAG | 518 | p7-110 | ACAGGAGCCA |
| TY008 | 111 | p5-111 | ACTGAGTAAC | 519 | p7-111 | CGTTCTACTT |
| TY360 | 112 | p5-112 | GTCCTAGTAA | 520 | p7-112 | ATGCAGATTC |
| TY199 | 113 | p5-113 | GATCTCGATA | 521 | p7-113 | TCCATGGCTG |
| TY345 | 114 | p5-114 | TGATCTTCGC | 522 | p7-114 | GTACCAAGAA |
| TY193 | 115 | p5-115 | TCTCAGCGCT | 523 | p7-115 | AGTGGTCAAT |
| TY346 | 116 | p5-116 | ACGAGAGTGG | 524 | p7-116 | CGTTACGTGG |
| TY323 | 117 | p5-117 | CGCGTCAGAA | 525 | p7-117 | TACTTACGCA |
| TY389 | 118 | p5-118 | GTCTCTGGAT | 526 | p7-118 | TGGCGTTAAC |
| TY382 | 119 | p5-119 | CAGTGGAATC | 527 | p7-119 | GAAGAGTACA |
| TY186 | 120 | p5-120 | ACTTGTCTGT | 528 | p7-120 | ATGACCATTC |
| TY415 | 121 | p5-121 | AGCTGTACAA | 529 | p7-121 | TGGTAGGAAG |
| TY057 | 122 | p5-122 | CTAGCATGAT | 530 | p7-122 | GTAGTTCGGA |
| TY059 | 123 | p5-123 | TAGATTCTCC | 531 | p7-123 | CCTAGTACAT |
| TY541 | 124 | p5-124 | ACATACGATG | 532 | p7-124 | CACTGCGTCT |
| TY218 | 125 | p5-125 | TGTCGGTGGA | 533 | p7-125 | TAACACCTTC |
| TY063 | 126 | p5-126 | CTGGATGCCA | 534 | p7-126 | ACGGCATTCT |
| TY442 | 127 | p5-127 | GCTAGACAGG | 535 | p7-127 | CTACTGTCTC |
| TY427 | 128 | p5-128 | AGCATGTTCC | 536 | p7-128 | AGGCGTAAGA |
| TY198 | 129 | p5-129 | TACGGAAGAA | 537 | p7-129 | TCATCCAGGA |
| TY465 | 130 | p5-130 | CGACTGACTC | 538 | p7-130 | AGCATTCTCG |
| TY493 | 131 | p5-131 | GCTTGCTTAT | 539 | p7-131 | CAACGTCCTC |
| TY136 | 132 | p5-132 | AGGCCACAGA | 540 | p7-132 | AGTGTGGAAT |
| TY183 | 133 | p5-133 | GACAATGAGT | 541 | p7-133 | GCGCAAGTCA |
| TY215 | 134 | p5-134 | ATCTCCGGTG | 542 | p7-134 | CTCAGCTCCA |
| TY359 | 135 | p5-135 | TCTACTCGCG | 543 | p7-135 | AACACATGGT |
| TY072 | 136 | p5-136 | AGAGTCTCTT | 544 | p7-136 | GATGCGAGAC |
| TY522 | 137 | p5-137 | CACTGCAATT | 545 | p7-137 | AATCGACCGA |
| TY520 | 138 | p5-138 | ACTGACTTGA | 546 | p7-138 | TCGGAGTTCC |
| TY024 | 139 | p5-139 | TAACCGGACC | 547 | p7-139 | CGATGGAGTG |
| TY240 | 140 | p5-140 | TCCATTCCAA | 548 | p7-140 | GTCTCAGTAG |
| TY470 | 141 | p5-141 | TTGTGAGGCG | 549 | p7-141 | TACACTTGCA |
| TY332 | 142 | p5-142 | GGAGAGTTAT | 550 | p7-142 | GTAATCGACG |
| TY398 | 143 | p5-143 | AAGGATAGCC | 551 | p7-143 | AGTGAGACGT |
| TY226 | 144 | p5-144 | TGTCCGGCTA | 552 | p7-144 | CACCTCCTAC |
| TY159 | 145 | p5-145 | CTGCTCGTCT | 553 | p7-145 | ACTTCCGTCC |
| TY137 | 146 | p5-146 | GCTAGTCGAA | 554 | p7-146 | AGCCAGTGGA |
| TY156 | 147 | p5-147 | TGAGAGTCGC | 555 | p7-147 | CAGTGATCGG |
| TY496 | 148 | p5-148 | AGATAACCTG | 556 | p7-148 | GTAACTAACC |
| TY498 | 149 | p5-149 | TTCGCGGAGT | 557 | p7-149 | TGTGCCTTAT |
| TY073 | 150 | p5-150 | CAGTATAGGA | 558 | p7-150 | TGAATACGTC |
| TY211 | 151 | p5-151 | TAGAGCATTC | 559 | p7-151 | GTCGGTAAGG |
| TY239 | 152 | p5-152 | ATCCTAACCT | 560 | p7-152 | CAAGTGAGTT |
| TY311 | 153 | p5-153 | GTTAAGTGGT | 561 | p7-153 | TAGGCCGATA |
| TY558 | 154 | p5-154 | CGGCTCCTTA | 562 | p7-154 | ACACTGTAGT |
| TY116 | 155 | p5-155 | TACTGAATCC | 563 | p7-155 | TGCGGTATTA |
| TY423 | 156 | p5-156 | ACAGCAACAA | 564 | p7-156 | TTGTAGTACG |
| TY220 | 157 | p5-157 | AGGAGTTAAC | 565 | p7-157 | AGTACAAGTC |
| TY326 | 158 | p5-158 | TAACAGGCTA | 566 | p7-158 | CTGTATCCAT |
| TY178 | 159 | p5-159 | CCTTGTCAGG | 567 | p7-159 | GACATCATTC |
| TY328 | 160 | p5-160 | TTGGTATGCT | 568 | p7-160 | GTTCTAAGAG |
| TY206 | 161 | p5-161 | AACGTAAGCA | 569 | p7-161 | AAGTCGAGAG |
| TY385 | 162 | p5-162 | CCACAGCTGT | 570 | p7-162 | TCCGGACTCA |
| TY067 | 163 | p5-163 | TATAGCGAAC | 571 | p7-163 | CTTAACACCT |
| TY210 | 164 | p5-164 | GGTCCTTCAC | 572 | p7-164 | GGTTATCTTC |
| TY355 | 165 | p5-165 | TTGCGAGATA | 573 | p7-165 | TGAGTAGATC |
| TY322 | 166 | p5-166 | GAATCCGACT | 574 | p7-166 | ATGCGGTGGT |
| TY213 | 167 | p5-167 | CTGATTCTAG | 575 | p7-167 | TAAGTTGCCT |
| TY160 | 168 | p5-168 | ACGGCGATTG | 576 | p7-168 | ACACACCATG |
| TY253 | 169 | p5-169 | TGACGGAGTA | 577 | p7-169 | AAGTGGTAGG |
| TY559 | 170 | p5-170 | ATTGATGAGC | 578 | p7-170 | TTGGACGGAC |
| TY091 | 171 | p5-171 | GGCTAGCCAT | 579 | p7-171 | GGACTTCGCT |
| TY093 | 172 | p5-172 | TATGCCTTCG | 580 | p7-172 | GATACAGCTA |
| TY549 | 173 | p5-173 | CCGATCCATT | 581 | p7-173 | CACACCGCAT |
| TY388 | 174 | p5-174 | GGTGTATAGA | 582 | p7-174 | CCTCCGATCT |
| TY196 | 175 | p5-175 | CTGTCTGCAC | 583 | p7-175 | AGTTGTCCTA |
| TY032 | 176 | p5-176 | TCACCAATCA | 584 | p7-176 | GTAGGCAATG |
| TY083 | 177 | p5-177 | TGAGAACGGT | 585 | p7-177 | TGACAACTCT |
| TY078 | 178 | p5-178 | GTCCTGAACA | 586 | p7-178 | ACGTGCAAGC |
| TY320 | 179 | p5-179 | ACGAGTTGCC | 587 | p7-179 | TATATCGGAG |
| TY432 | 180 | p5-180 | CATTAAGCTG | 588 | p7-180 | AATACTTCCG |
| TY403 | 181 | p5-181 | CTGCACCTAC | 589 | p7-181 | GTGGTATCAA |
| TY079 | 182 | p5-182 | CGCTCTGATG | 590 | p7-182 | CTCACAGATA |
| TY305 | 183 | p5-183 | GCTGACTATT | 591 | p7-183 | CGACTGATGT |
| TY142 | 184 | p5-184 | TACTTGGCTA | 592 | p7-184 | GGCTCTTGTC |
| TY536 | 185 | p5-185 | CATCAGCCAA | 593 | p7-185 | AACCTACAAC |
| TY296 | 186 | p5-186 | GCCTTGTATT | 594 | p7-186 | TGTTATTGCG |
| TY007 | 187 | p5-187 | TTGACTATCG | 595 | p7-187 | TACACGATCA |
| TY329 | 188 | p5-188 | AATTGTAGGC | 596 | p7-188 | ACTGACGCGT |
| TY306 | 189 | p5-189 | AGGTCCGTAG | 597 | p7-189 | GTATGCGGTC |
| TY225 | 190 | p5-190 | CAAGAACACC | 598 | p7-190 | CCTGACTACG |
| TY454 | 191 | p5-191 | CCTAGATGTA | 599 | p7-191 | CGGATACAGG |
| TY107 | 192 | p5-192 | TGCTTCGAAT | 600 | p7-192 | ATACGTAGGA |
| TY515 | 193 | p5-193 | AAGCTTGGAT | 601 | p7-193 | TGTATCAGAC |
| TY084 | 194 | p5-194 | TCCTGGTTCC | 602 | p7-194 | CTGCCTTCCT |
| TY126 | 195 | p5-195 | GCTATCAAGA | 603 | p7-195 | ACCTTCCAGG |
| TY066 | 196 | p5-196 | GTCGGACTGT | 604 | p7-196 | GAAGGATTGA |
| TY517 | 197 | p5-197 | CCATAACCTT | 605 | p7-197 | ATAAGGCTTG |
| TY113 | 198 | p5-198 | AGTACTCCGC | 606 | p7-198 | GATCAGGCCT |
| TY336 | 199 | p5-199 | TTGGCGGTTG | 607 | p7-199 | GAATGAAGAC |
| TY069 | 200 | p5-200 | AGTTATAGCG | 608 | p7-200 | ACGCTTGATG |
| TY146 | 201 | p5-201 | TGACGCGGAT | 609 | p7-201 | ATCATCACGT |
| TY089 | 202 | p5-202 | CAGAATAACC | 610 | p7-202 | CCAGGTTGAC |
| TY074 | 203 | p5-203 | ATGGCACGGA | 611 | p7-203 | GATCAGTTCA |
| TY268 | 204 | p5-204 | GGCAGCTTAA | 612 | p7-204 | AGGTGACATC |
| TY114 | 205 | p5-205 | CCTGTTGTGT | 613 | p7-205 | TTCTCGGAAG |
| TY353 | 206 | p5-206 | CACTCGACCA | 614 | p7-206 | GCACCACCAA |
| TY104 | 207 | p5-207 | ACACGTCGTG | 615 | p7-207 | TTGATGGCGG |
| TY418 | 208 | p5-208 | GGTATGCACC | 616 | p7-208 | GGACATTGTT |
| TY519 | 209 | p5-209 | TTGAACCGCT | 617 | p7-209 | ACAGCAGATG |
| TY356 | 210 | p5-210 | CGAACTAAGC | 618 | p7-210 | GGCCTTAGAA |
| TY405 | 211 | p5-211 | GGTTAAGCTT | 619 | p7-211 | TTGAGGTAAC |
| TY556 | 212 | p5-212 | GGCGGATGAA | 620 | p7-212 | AGATAACGCT |
| TY151 | 213 | p5-213 | TTGCTGATTG | 621 | p7-213 | TGTTGCATGC |
| TY463 | 214 | p5-214 | CCTCATTAGA | 622 | p7-214 | CAGCACACCT |
| TY361 | 215 | p5-215 | AAGGCGACCA | 623 | p7-215 | GAGAGGTCCA |
| TY464 | 216 | p5-216 | ATCTGTCCGC | 624 | p7-216 | ATGGTTGTGG |
| TY341 | 217 | p5-217 | CGGTAATGAT | 625 | p7-217 | TTCACCTGCT |
| TY284 | 218 | p5-218 | CTCGTTGCGA | 626 | p7-218 | AGATGTCAGA |
| TY294 | 219 | p5-219 | GCACACATGA | 627 | p7-219 | TAGGTGGCTG |
| TY308 | 220 | p5-220 | TACAATCCTC | 628 | p7-220 | GCTAAGTTAC |
| TY016 | 221 | p5-221 | TCTCCGTACC | 629 | p7-221 | CGCTCGAGTT |
| TY297 | 222 | p5-222 | AACGTCTAAG | 630 | p7-222 | CCTCGAATGA |
| TY386 | 223 | p5-223 | AGTGGCGTCT | 631 | p7-223 | ATAATCTCGG |
| TY397 | 224 | p5-224 | GTCTTAGGTT | 632 | p7-224 | TAGAACGAAC |
| TY546 | 225 | p5-225 | AATAGCTGCA | 633 | p7-225 | TCACTAACGA |
| TY018 | 226 | p5-226 | GTCTCAGAAG | 634 | p7-226 | ATCACTTGAC |
| TY201 | 227 | p5-227 | TCAGTGCTCC | 635 | p7-227 | GGTGACCAGT |
| TY202 | 228 | p5-228 | CGTCATACTT | 636 | p7-228 | GATCGTATTC |
| TY122 | 229 | p5-229 | TCGGATTGGC | 637 | p7-229 | TTGTCTGATG |
| TY528 | 230 | p5-230 | AATGGAGCAA | 638 | p7-230 | CATACGGACC |
| TY097 | 231 | p5-231 | CGGTTCATGT | 639 | p7-231 | GCCGGTCTAT |
| TY530 | 232 | p5-232 | GCCAGTTATT | 640 | p7-232 | AGAATACCGA |
| TY033 | 233 | p5-233 | CTGCGTTACA | 641 | p7-233 | AGTAAGATGG |
| TY469 | 234 | p5-234 | TGAGTAGCAT | 642 | p7-234 | TGAGGACCAA |
| TY166 | 235 | p5-235 | GACTCGTTGA | 643 | p7-235 | CTGTAATGTC |
| TY221 | 236 | p5-236 | CTACATCCGG | 644 | p7-236 | GACCTTGACT |
| TY207 | 237 | p5-237 | GATACCGGTC | 645 | p7-237 | ACCTACTCTA |
| TY257 | 238 | p5-238 | ACACCTAATG | 646 | p7-238 | CCAACGTATA |
| TY304 | 239 | p5-239 | ACCTTGTGCT | 647 | p7-239 | TTAGTTCACG |
| TY262 | 240 | p5-240 | TAGGTAGTAC | 648 | p7-240 | TGTTCCGAGC |
| TY472 | 241 | p5-241 | CACCGTCGAA | 649 | p7-241 | ATGTTGACGT |
| TY290 | 242 | p5-242 | TTGAAGGTTC | 650 | p7-242 | GCAAGATAAC |
| TY214 | 243 | p5-243 | GCCTTAACCA | 651 | p7-243 | TAGGCAGGAG |
| TY102 | 244 | p5-244 | TAAGCGTATC | 652 | p7-244 | CCTCATTCTT |
| TY283 | 245 | p5-245 | GATATGAAGG | 653 | p7-245 | AGCAACCGCA |
| TY045 | 246 | p5-246 | AGTGTCCATT | 654 | p7-246 | GATGTCTATC |
| TY509 | 247 | p5-247 | CCTGAACTGT | 655 | p7-247 | TGAACGCTTA |
| TY014 | 248 | p5-248 | CGATGTGCAA | 656 | p7-248 | TAGCGCGAAG |
| TY127 | 249 | p5-249 | CGAGACCAGT | 657 | p7-249 | AGTCGAAGCC |
| TY090 | 250 | p5-250 | ATTAGAGGAC | 658 | p7-250 | TCGTCGCCAA |
| TY456 | 251 | p5-251 | TACCTGACGC | 659 | p7-251 | ATCGCTGTCT |
| TY333 | 252 | p5-252 | CCGTCATCAA | 660 | p7-252 | TGCAACCATT |
| TY286 | 253 | p5-253 | GGACTCATAT | 661 | p7-253 | CAGGAGTTGG |
| TY507 | 254 | p5-254 | ATTCTTGGTG | 662 | p7-254 | TGAGTCTCGC |
| TY281 | 255 | p5-255 | AACAACTCCA | 663 | p7-255 | GTTACAACAC |
| TY144 | 256 | p5-256 | CTTGCACATT | 664 | p7-256 | GCAATAGATG |
| TY115 | 257 | p5-257 | TGCTTGGTCA | 665 | p7-257 | AAGAGCCTGA |
| TY471 | 258 | p5-258 | GCTCATTCAT | 666 | p7-258 | GAACAAGCCG |
| TY557 | 259 | p5-259 | CTCACAACTT | 667 | p7-259 | CGCACTTATT |
| TY162 | 260 | p5-260 | TCAGGCCATG | 668 | p7-260 | TGCATGACGC |
| TY175 | 261 | p5-261 | TACCATTGGA | 669 | p7-261 | GCTGAAGGAA |
| TY125 | 262 | p5-262 | AAGTTAGCTC | 670 | p7-262 | GTTGACGTTA |
| TY510 | 263 | p5-263 | GTTAGGATAC | 671 | p7-263 | CTCTATTGAG |
| TY260 | 264 | p5-264 | AGGAGCCACA | 672 | p7-264 | AAGTGCCGTC |
| TY307 | 265 | p5-265 | TGACGCACAA | 673 | p7-265 | ACGACAACCA |
| TY425 | 266 | p5-266 | CCTTACGATT | 674 | p7-266 | CTATTGCTAC |
| TY378 | 267 | p5-267 | ATTGTACTCC | 675 | p7-267 | TCGTTCTCGG |
| TY263 | 268 | p5-268 | TAGGACTCGA | 676 | p7-268 | GATGATAGGT |
| TY462 | 269 | p5-269 | TCCAGTTGCG | 677 | p7-269 | TACAAGGAGA |
| TY410 | 270 | p5-270 | GGAATGAACT | 678 | p7-270 | GTCCGCAGTT |
| TY128 | 271 | p5-271 | GAACCAAGAA | 679 | p7-271 | AGAGTATGCT |
| TY319 | 272 | p5-272 | CTCTTCCATT | 680 | p7-272 | CGACCGGTTA |
| TY099 | 273 | p5-273 | GGATACGCAA | 681 | p7-273 | ATATGGACGA |
| TY504 | 274 | p5-274 | AACCGTCATT | 682 | p7-274 | AGAATTGTCC |
| TY312 | 275 | p5-275 | TCGTCTTGCC | 683 | p7-275 | TATGTTCCAG |
| TY426 | 276 | p5-276 | AGAGTGCGCA | 684 | p7-276 | TGCCAATGTT |
| TY534 | 277 | p5-277 | GTCTTAATGG | 685 | p7-277 | ATGTCCGTGA |
| TY411 | 278 | p5-278 | CTTACCTTCT | 686 | p7-278 | CCGGTCACTA |
| TY026 | 279 | p5-279 | GAGTTAGAAG | 687 | p7-279 | GCCAGTTAAT |
| TY412 | 280 | p5-280 | CAACGACCTA | 688 | p7-280 | AATACAGACG |
| TY155 | 281 | p5-281 | GGTATTGGAA | 689 | p7-281 | AGCAATCAAC |
| TY514 | 282 | p5-282 | CAAGCGAATT | 690 | p7-282 | TATCGCGCGT |
| TY288 | 283 | p5-283 | TGGTGCCACT | 691 | p7-283 | CCTTAATTCG |
| TY255 | 284 | p5-284 | CTTGTGTTGA | 692 | p7-284 | GAAGTTAGCA |
| TY488 | 285 | p5-285 | ATGCAACGTT | 693 | p7-285 | TTGGCCTAGG |
| TY379 | 286 | p5-286 | TCGCATGCAC | 694 | p7-286 | TGTTAGGTAC |
| TY264 | 287 | p5-287 | TTCAGTGTGG | 695 | p7-287 | TTCCTTGTTG |
| TY038 | 288 | p5-288 | GAATGATCAC | 696 | p7-288 | ACTCCACGTA |
| TY272 | 289 | p5-289 | TTGAGACTGA | 697 | p7-289 | ATTCAGAGTG |
| TY106 | 290 | p5-290 | GCTTATGTCT | 698 | p7-290 | TCGACATTGC |
| TY227 | 291 | p5-291 | GAAGCCAGGT | 699 | p7-291 | GGATGCTCGT |
| TY495 | 292 | p5-292 | TACCAGCCAC | 700 | p7-292 | CAACTAGGAA |
| TY357 | 293 | p5-293 | CGAATCTGTT | 701 | p7-293 | CCTGTTCAGT |
| TY010 | 294 | p5-294 | ATAGCACACT | 702 | p7-294 | AACGTCTACC |
| TY368 | 295 | p5-295 | TTGCTAGCAG | 703 | p7-295 | TTGAGCATAC |
| TY279 | 296 | p5-296 | CGGTCTTATT | 704 | p7-296 | AACTGTGCTT |
| TY223 | 297 | p5-297 | ATGTTCGCAA | 705 | p7-297 | ATTGGCTGAA |
| TY161 | 298 | p5-298 | CATGACAACC | 706 | p7-298 | GAACCTATCT |
| TY452 | 299 | p5-299 | AGACTTCTGG | 707 | p7-299 | TCGGCGCATA |
| TY384 | 300 | p5-300 | TCCATCTGTA | 708 | p7-300 | GTCACTACTG |
| TY068 | 301 | p5-301 | GAACGGTTAT | 709 | p7-301 | CGGTGTGCAT |
| TY550 | 302 | p5-302 | TGTCAAGTTC | 710 | p7-302 | AGACAATTGG |
| TY065 | 303 | p5-303 | CCGTCAAGGA | 711 | p7-303 | TGACTGCGAA |
| TY139 | 304 | p5-304 | TGGAGGTCCT | 712 | p7-304 | TCAAGCCACC |
| TY363 | 305 | p5-305 | TTCCAAGCAA | 713 | p7-305 | TAATCAGCTG |
| TY374 | 306 | p5-306 | CAGAGGTATT | 714 | p7-306 | GCAGATTGCA |
| TY443 | 307 | p5-307 | AGATCTAGCA | 715 | p7-307 | ATGGAACTGT |
| TY406 | 308 | p5-308 | AAGGTACTGT | 716 | p7-308 | CGTACGACGA |
| TY096 | 309 | p5-309 | GCACTTGGTC | 717 | p7-309 | ATTATCGAGG |
| TY019 | 310 | p5-310 | CGTATCCTGG | 718 | p7-310 | TAATTCCTGC |
| TY030 | 311 | p5-311 | GTAGAATCAC | 719 | p7-311 | AGCCAACGAC |
| TY402 | 312 | p5-312 | CCTAGGAACT | 720 | p7-312 | CTAGGCGGTT |
| TY527 | 313 | p5-313 | TTACAGCGTT | 721 | p7-313 | TCCAGACGAG |
| TY364 | 314 | p5-314 | CGCTGTAAGC | 722 | p7-314 | CGTCTCAACC |
| TY219 | 315 | p5-315 | GAGGCCATAT | 723 | p7-315 | GTATGTACGT |
| TY478 | 316 | p5-316 | ATTGGAACCG | 724 | p7-316 | AACACGCTAA |
| TY163 | 317 | p5-317 | GCTATAGCTA | 725 | p7-317 | GGCGATGAAG |
| TY212 | 318 | p5-318 | AGAGGTTCGT | 726 | p7-318 | CGGATATGTA |
| TY512 | 319 | p5-319 | AACCTCTTCA | 727 | p7-319 | AAGGTTATGC |
| TY208 | 320 | p5-320 | CCATAAGAGT | 728 | p7-320 | ACATCGGTGC |
| TY165 | 321 | p5-321 | AGGTGTTGAT | 729 | p7-321 | TTCAGACCGT |
| TY408 | 322 | p5-322 | TCCGAGGATC | 730 | p7-322 | ACAGATCTCC |
| TY189 | 323 | p5-323 | GAAGCACTAT | 731 | p7-323 | TAACTCTGAG |
| TY537 | 324 | p5-324 | ATTGCAGCCA | 732 | p7-324 | GGTACGAATT |
| TY497 | 325 | p5-325 | CGGATTAAGA | 733 | p7-325 | ATGAGCTAGA |
| TY310 | 326 | p5-326 | CTACTACAGA | 734 | p7-326 | CGTTAAGCAT |
| TY413 | 327 | p5-327 | GGTCACATGG | 735 | p7-327 | TTGTGTTCCT |
| TY080 | 328 | p5-328 | GCGATCTCAC | 736 | p7-328 | ACTGTGAGCG |
| TY526 | 329 | p5-329 | GTCATGTCGT | 737 | p7-329 | ATATGTGTGG |
| TY524 | 330 | p5-330 | TGGCTTATCC | 738 | p7-330 | GACATGTCAT |
| TY046 | 331 | p5-331 | TATTGAGCGT | 739 | p7-331 | TAGCCACATA |
| TY039 | 332 | p5-332 | ATCGCCAGAA | 740 | p7-332 | CGAGGATCAC |
| TY216 | 333 | p5-333 | CCTCAACATT | 741 | p7-333 | ACGCAGTTAG |
| TY190 | 334 | p5-334 | TAAGTCAGTG | 742 | p7-334 | TCACGGAGCT |
| TY237 | 335 | p5-335 | CGCTAGGTAT | 743 | p7-335 | CTTCACAACG |
| TY484 | 336 | p5-336 | CCATGCCTCA | 744 | p7-336 | CGTTATGAGT |
| TY238 | 337 | p5-337 | CAGGAATTGA | 745 | p7-337 | AGAACAGCGT |
| TY070 | 338 | p5-338 | TCAATGCAAC | 746 | p7-338 | TCCTGCAAGG |
| TY532 | 339 | p5-339 | AGACCTATCT | 747 | p7-339 | CTTCAGTCAA |
| TY234 | 340 | p5-340 | TATTCCGATC | 748 | p7-340 | AATAAGCTCC |
| TY543 | 341 | p5-341 | GTCCGTAAGA | 749 | p7-341 | GAAGGCGGAA |
| TY232 | 342 | p5-342 | TCTTGTCCAA | 750 | p7-342 | ACGATTCGAA |
| TY235 | 343 | p5-343 | GTAACGAGCT | 751 | p7-343 | TCCTGCTCTT |
| TY203 | 344 | p5-344 | TGGTGCTTGG | 752 | p7-344 | CTCGAACACG |
| TY347 | 345 | p5-345 | TGCCACAATT | 753 | p7-345 | ACTGTTACAC |
| TY275 | 346 | p5-346 | GCTTCTATGA | 754 | p7-346 | TGACACCACA |
| TY492 | 347 | p5-347 | CAATTGTTCC | 755 | p7-347 | GGTAAGGTCG |
| TY380 | 348 | p5-348 | GTTGCCTAGA | 756 | p7-348 | CTGTCGAGGT |
| TY054 | 349 | p5-349 | TATCAAGCGG | 757 | p7-349 | AAGAGATAGC |
| TY249 | 350 | p5-350 | ATTAGTCGTC | 758 | p7-350 | CCATTACCAA |
| TY485 | 351 | p5-351 | GGTCTAACAT | 759 | p7-351 | CACGGACTTC |
| TY479 | 352 | p5-352 | TGGCAGTAAT | 760 | p7-352 | GTACATTACG |
| TY141 | 353 | p5-353 | CTCTAGTGAT | 761 | p7-353 | TAGGAGACAA |
| TY042 | 354 | p5-354 | TAGCTTGACC | 762 | p7-354 | AGATCTATCG |
| TY181 | 355 | p5-355 | GGAGCAACTG | 763 | p7-355 | TTACTGTGCG |
| TY334 | 356 | p5-356 | TATCACCTCA | 764 | p7-356 | CCGAATCCTC |
| TY233 | 357 | p5-357 | ACGGAACAGG | 765 | p7-357 | GCTGGATTAA |
| TY499 | 358 | p5-358 | GCTCGAGGTA | 766 | p7-358 | ACCGTCTCGT |
| TY292 | 359 | p5-359 | CTGATTAGCC | 767 | p7-359 | AGAGCGGAGA |
| TY006 | 360 | p5-360 | CACTGCGCAA | 768 | p7-360 | GAACACGGAG |
| TY365 | 361 | p5-361 | GGCATCTATT | 769 | p7-361 | AGTCTTGTGA |
| TY441 | 362 | p5-362 | CTATGAGGAA | 770 | p7-362 | TTAAGGCGAG |
| TY494 | 363 | p5-363 | ATGTCTACCA | 771 | p7-363 | TCGTCAATTC |
| TY036 | 364 | p5-364 | TGTTAGGTGA | 772 | p7-364 | CACCACTTGT |
| TY367 | 365 | p5-365 | CCTGATCGCA | 773 | p7-365 | TTCGCAGACT |
| TY273 | 366 | p5-366 | AACAAGAACG | 774 | p7-366 | GAGACTCCGT |
| TY108 | 367 | p5-367 | TACCGAACAC | 775 | p7-367 | TGCGGAGAAC |
| TY362 | 368 | p5-368 | CATTGCTTGC | 776 | p7-368 | ACATATCGCG |
| TY261 | 369 | p5-369 | CGATTGAGTT | 777 | p7-369 | ATCAGGACAG |
| TY371 | 370 | p5-370 | GAGCATGCAA | 778 | p7-370 | GAATTGCGTT |
| TY490 | 371 | p5-371 | TCAGGTTAGC | 779 | p7-371 | TGTGGAGCCT |
| TY521 | 372 | p5-372 | GGTTCAATCA | 780 | p7-372 | AGGAACAAGA |
| TY316 | 373 | p5-373 | AGCAAGCTGC | 781 | p7-373 | CCAAGCTTCT |
| TY236 | 374 | p5-374 | GAACGCTGTC | 782 | p7-374 | TGGCATTGGC |
| TY351 | 375 | p5-375 | ATTCGTCATG | 783 | p7-375 | ACAACGCGGT |
| TY395 | 376 | p5-376 | CAGATCCTAA | 784 | p7-376 | GAGCTACCAC |
| TY003 | 377 | p5-377 | TGCATCCTGA | 785 | p7-377 | TTGTAACCAG |
| TY110 | 378 | p5-378 | GAGGAGAATT | 786 | p7-378 | GCACTATTCT |
| TY376 | 379 | p5-379 | TCTCGATGAA | 787 | p7-379 | ATGGCCAACA |
| TY289 | 380 | p5-380 | AGCGCATAAC | 788 | p7-380 | CATACTACTC |
| TY348 | 381 | p5-381 | CCAATTACCA | 789 | p7-381 | AGCCTGTCCA |
| TY314 | 382 | p5-382 | AGTTCCGGAA | 790 | p7-382 | TCATGTCGGT |
| TY535 | 383 | p5-383 | TTAAGGAAGC | 791 | p7-383 | ACAGGTGGAG |
| TY229 | 384 | p5-384 | CGCAATGTGG | 792 | p7-384 | CAACTCAACT |
| TY217 | 385 | p5-385 | TAAGAAGGCC | 793 | p7-385 | ACTTAGTAGC |
| TY204 | 386 | p5-386 | ATGTGTTCGT | 794 | p7-386 | AGGATATCCA |
| TY458 | 387 | p5-387 | GTTCACCACT | 795 | p7-387 | GCATCAAGAT |
| TY473 | 388 | p5-388 | CCGACGATGA | 796 | p7-388 | AGGAATGTTC |
| TY062 | 389 | p5-389 | AGAATAGAGG | 797 | p7-389 | TTCTACTAGC |
| TY123 | 390 | p5-390 | CTCATTGTCA | 798 | p7-390 | CAGAGGCTAT |
| TY088 | 391 | p5-391 | GCATTCGCTA | 799 | p7-391 | ATTGTGAAGG |
| TY487 | 392 | p5-392 | TGTGAGCTAT | 800 | p7-392 | GTCCTTAACA |
| TY012 | 393 | p5-393 | GAACATAGGT | 801 | p7-393 | AGGCGAGCTT |
| TY034 | 394 | p5-394 | TGGTTGGATA | 802 | p7-394 | TGCTCTCGAT |
| TY483 | 395 | p5-395 | AACACCTGGT | 803 | p7-395 | GAATGGTTCA |
| TY317 | 396 | p5-396 | TATCGATTCG | 804 | p7-396 | ATCGAGAATC |
| TY414 | 397 | p5-397 | TCATTACAGC | 805 | p7-397 | CCAACATTGA |
| TY209 | 398 | p5-398 | TTAGAGCTCA | 806 | p7-398 | ATTCTCCAGT |
| TY315 | 399 | p5-399 | TTGGTGACAA | 807 | p7-399 | GGCATTATCA |
| TY187 | 400 | p5-400 | CTGTGATATC | 808 | p7-400 | ATCGTACATG |
| TY460 | 401 | p5-401 | CAAGTGGTCT | 809 | p7-401 | TGTCTACGGC |
| TY095 | 402 | p5-402 | ATGACTAGGA | 810 | p7-402 | AGAACCAATG |
| TY023 | 403 | p5-403 | TACTGTCGTA | 811 | p7-403 | GTCGACGACA |
| TY143 | 404 | p5-404 | ACACACAAGG | 812 | p7-404 | CTTGGTATGT |
| TY516 | 405 | p5-405 | TGCCTAGCGT | 813 | p7-405 | GACTTATCCT |
| TY058 | 406 | p5-406 | GCTATCCTCT | 814 | p7-406 | GCGTGAATCA |
| TY044 | 407 | p5-407 | TGCTAGTTGT | 815 | p7-407 | CTCCTGTTAT |
| TY138 | 408 | p5-408 | GTACACGGAC | 816 | p7-408 | ATATTAGCGC |

Further, performing library construction on the target sample utilizing the primer with 5′ phosphorylation modification, obtaining the linear amplification library with 5′ phosphorylation modification includes the following operations.

Adapter ligation is performed on a fragment derived from the above target sample utilizing truncated adapters shown in SEQ ID NO: 817 and SEQ ID NO: 818, obtaining a fragment with adapters.

The above fragment with adapters is amplified utilizing the above P5 truncated amplification primer and the above P7 truncated amplification primer, obtaining the above linear amplification library with 5′ phosphorylation modification.

a sequence of SEQ ID NO: 817 is
5′-ACACTCTTTCCCTACACGACGCTCTTCCGATC*T-3′, and * represents thio modification;
and
a sequence of SEQ ID NO: 818 is
5′-GATCGGAAGAGCACACGTCTGAACTCCAGTCAC-3′, and a 5′ end is modified through
phosphorylation.

Further, performing library construction on the target sample utilizing the above adapter with 5′ phosphorylation modification, obtaining the linear amplification library with 5′ phosphorylation modification includes the following operations.

Adapter ligation is performed on a fragment derived from the above target sample utilizing the above P5 full-length adapter and the above P7 full-length adapter, obtaining a fragment with adapters.

The above fragment with the adapters is amplified utilizing library amplification primers shown in SEQ ID NO: 822 and SEQ ID NO: 825, obtaining the above linear amplification library with 5′ phosphorylation modification.

a sequence of SEQ ID NO: 822 is
5′-AATGATACGGCGACCACCGAGAT-3′, and a 5′ end is modified through phosphorylation;
and
a sequence of SEQ ID NO: 825 is
5′-CAAGCAGAAGACGGCATACGA-3′.

Further, before circularization, the above method for constructing a DNA library further includes a step of performing targeted capture on the above linear amplification library.

Further, the captured library after targeted capture is amplified utilizing the above targeted library amplification primers, obtaining a linear amplified captured library; and the above linear amplified captured library is circularized to obtain the above circularized library suitable for the MGI® sequencing platform.

Further, the above targeted library amplification primers have nucleotide sequences shown in SEQ ID NO: 822 and SEQ ID NO: 825.

a sequence of SEQ ID NO: 822 is
5′-AATGATACGGCGACCACCGAGAT-3′, and a 5′ end is modified through phosphorylation;
and
a sequence of SEQ ID NO: 825 is
5′-CAAGCAGAAGACGGCATACGA-3′.

In order to implement the above objective, a second aspect of the present disclosure provides a kit for constructing a DNA library. The above kit for constructing a DNA library includes any one of the following combinations.
1) Combination 1: a P5 truncated amplification primer in the above method for constructing a DNA library and a P7 truncated amplification primer in the above method for constructing a DNA library.
2) Combination 2: a P5 full-length adapter in the above method for constructing a DNA library and a P7 full-length adapter in the above method for constructing a DNA library.

The above kit for constructing a DNA library includes 408 P5-end index sequences and 408 corresponding fixedly-matched P7-end index sequences, the above P5-end index sequences are shown in Table 1 in the above method for constructing a DNA library, and the above P7-end index sequences are shown in Table 1 in the above method for constructing a DNA library.

The above P5-end index sequence is used in conjunction with a group of 8-base balanced index sequences, and the above P7-end index sequence is used according to the fixedly-matched P7-end index sequences corresponding to the above P5-end index sequences.

Further, the kit for constructing a DNA library further includes library amplification primers shown in SEQ ID NO: 822 and SEQ ID NO: 825, and/or truncated adapters shown in SEQ ID NO: 817 and 818.

In order to implement the above objective, a third aspect of the present disclosure provides an adapter element compatible with dual-sequencing platforms. The above adapter element is selected from any one of the following combinations.
1) Combination 1: a P5 truncated amplification primer in the above method for constructing a DNA library and a P7 truncated amplification primer in the above method for constructing a DNA library.
2) Combination 2: a P5 full-length adapter in the above method for constructing a DNA library and a P7 full-length adapter in the above method for constructing a DNA library.

The sequences, which are 1 bp from upstream and downstream of the above index sequence including the above P5-end index sequence or the above P7-end index sequence, contain at least three edit distances.

The above adapter element is an amplification primer composition or an adapter composition. The above amplification primer composition includes a combination of the above P5 truncated amplification primers and/or the above P7 truncated amplification primers; the above P5 truncated amplification primers and the above P7 truncated amplification primers are each independently of a group or a plurality of groups; each group of the above P5 truncated amplification primers includes P5-end index sequences selected from any one of loading combinations in Table 1 in the above method for constructing a DNA library; each group of the above P7 truncated amplification primers includes fixedly-matched P7-end index sequences corresponding to the above P5-end index sequences selected from Table 1 in the above method for constructing a DNA library; the respective number of bases A, T, C, and G in the above loading combination is ≥12.5%.

The above adapter composition includes a plurality of groups of the above P5 full-length adapters and/or a plurality of groups of the above P7 full-length adapters; the above P5 full-length adapters and the above P7 full-length adapters are each independently of a group or a plurality of groups; each group of the above P5 full-length adapters includes P5-end index sequences selected from any one of loading combinations in Table 1 in the above method for constructing a DNA library; each group of the above P7 full-length adapters includes fixedly-matched P7-end index sequences corresponding to the above P5-end index sequences selected from Table 1 in the above method for constructing a DNA library; the respective number of bases A, T, C, and G in the above loading combination is ≥12.5%; and 8 indexes constitute a group of the above loading combinations. Utilizing the technical solutions of the present disclosure, based on three indicators, which are library output, whole-genome library sequencing data splitting, and captured library sequencing data splitting, all of which simultaneously meet the standard of differences in normalization less than +15%, double-ended fixedly-matched unique tag adapter combinations of various fixedly-matched P5 ends and P7 ends are screened out. Furthermore, in order to take low-throughput sequencing modes or the requirements for high sequencing volumes with small sample sizes into consideration, the present disclosure further arranges 51 loading combinations in groups of 8. The tag combination modes of the present disclosure can better meet the loading problem of the current dual sequencing platforms from Illumina® sequencing platform and MGI® sequencing platform and other sequencing platforms compatible with tags of the present disclosure, and meet the increasing requirements for the types and quality of sequencing adapters due to the ever-increasing throughput of current high-throughput sequencers, thereby facilitating balanced output control of simultaneous loading of a plurality of samples, thus further facilitating large-scale production.

### BRIEF DESCRIPTION OF DRAWINGS

The drawings, which form a part of the present disclosure, are used to provide a further understanding of the present disclosure. The exemplary embodiments of the present disclosure and the description thereof are used to explain the present disclosure, but do not constitute improper limitations to the present disclosure. In the drawings:

FIG. 1 is a schematic diagram of adverse factors affecting library output and data splitting, including an adapter as shown in SEQ ID NO: 826 producing a secondary structure that does not facilitate amplification.

FIG. 2 shows normalized values of 72 groups of 4-base balanced combinations provided in Patent CN113999893B based on data splitting of an Illumina® sequencing platform.

FIG. 3 is a schematic diagram of library output, whole-genome library sequencing data splitting, and captured library sequencing data splitting for 1-192 groups.

FIG. 4 is a schematic diagram of library output, whole-genome library sequencing data splitting, and captured library sequencing data splitting for 193-384 groups.

FIG. 5 is a schematic diagram of library output, whole-genome library sequencing data splitting, and captured library sequencing data splitting for 385-560 groups.

FIG. 6 is a schematic diagram of a sum of three pieces of normalized data (library output, whole-genome library sequencing data splitting, and captured library sequencing data splitting).

### DESCRIPTION OF EMBODIMENTS

It is to be noted that the embodiments in the disclosure and the features in the embodiments may be combined with one another without conflict. The present disclosure will be described below in detail with reference to the embodiments.

### Term Explanation:

Library output normalization: the same sample is broken into fragments with lengths of 200-400 bp through ultrasound, and 10 ng of broken DNA is introduced for library construction; except for index, all other operating conditions are consistent; and finally, a value obtained by dividing an output of a library by an average value of all libraries is referred to as a normalized value of the library, and it is ideal when the value is equal to 1.

Whole-genome sequencing data splitting normalization: libraries in which library inputs, amplification cycle numbers, and all operating conditions are consistent except for the index are used, different libraries constructed by different indexes are mixed with equal mass and then sequenced; generally, 0.2-0.5 GB of data is arranged for each index; data outputted through the sequencing of each library is normalized; a value obtained by dividing data output of a library by an average value of all libraries is referred to as a normalized value of the whole-genome sequencing data splitting; and it is ideal when the value is equal to 1.

Targeted capture data splitting normalization: the libraries with the same library inputs and construction parameters have different indexes. The libraries are mixed with equal mass, followed by targeted capture, and then the libraries after targeted capture are sequenced, obtaining sequencing data; generally, according to 0.2-05 GB of data for each library. A value obtained by dividing data of a library by an average value of all libraries is referred to as a normalized value; and it is ideal when the value is equal to 1.

Four-color channel and two-color channel of sequencer: referring to types and number of fluorescent markers used by the sequencer. These fluorescent markers emit different colors of fluorescence signals when DNA fragments are synthesized, so as to help the sequencer to identify sequences of the DNA fragments. Compared to the two-color channel, the four-color channel has higher diversity and sensitivity, and may simultaneously detect more different bases, thereby improving the accuracy and efficiency of DNA sequencing.

Edit distance: an indicator for measuring a difference degree between two sequences. The edit distance represents a minimum number of operations that is required by converting one sequence into another sequence, and may be implemented by means of inserting, deleting, or replacing characters. If the edit distance is smaller, it represents that two sequences are more similar; and if the edit distance is larger, it represents that the two sequences are less similar. The edit distance is widely applied to the field of bioinformatics for similarity analysis and comparison of DNA sequences, protein sequences, etc.

Library index adapters: also known as index adapters, it refers to adapters used for constructing sequencing libraries, wherein the sequence of the adapter contains the sequence of the indexes. The indexes can be used to distinguish between different samples under test.

4-base balanced index sequences: a group of 4 index sequences wherein, at each position from the first to the tenth in the index sequence, there is one base of A, one base of T, one base of C, and one base of G.

In order to further improve sequencing quality, the previous research of the inventor screened various 4-base balanced P5-end index sequences and P7-end index sequences from two dimensions: library output and data splitting, and disclosed the above sequences in Patent CN113999893B. Provided in the patent was a library construction method compatible with dual-sequencing platforms (Illumina® sequencing platform and MGI® sequencing platform). The research thought and application values of the present disclosure are introduced below in detail with reference to the research and development background of the Patent CN113999893B.

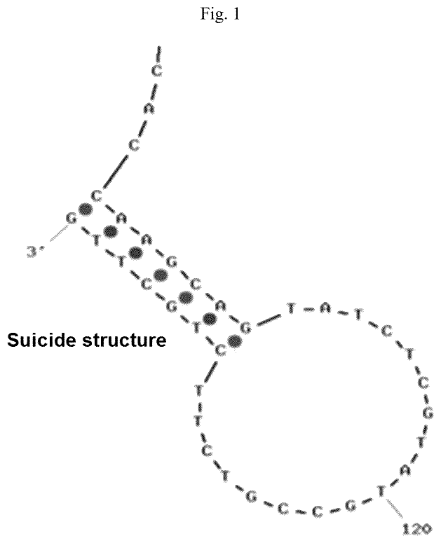

As the application became more widespread, the inventor discovered in practical application that when clients used index sequences for sequencing experiments, they not only considered library output, but also the output of effective data after data splitting. However, before the Patent CN113999893B was proposed, even Integrated DNA Technologies (IDT®) Company—a company recognized for its high-quality synthetic NGS adapters—did not consider removing unfavorable factors affecting library output and data splitting when designing index products, despite referencing the earliest article on index screening via algorithms: Somervuo et al. BMC Bioinformatics (2018) 19:257. As a result, the library output of a product-No. 201 adapter developed by the company was only 30% of an average of other adapters (other adapters produced by other companies that had assessed factors affecting amplification). Therefore, to enhance sequencing quality, clients may need to evaluate index products themselves after purchasing them from the market. This is to identify and exclude indices with low library output and poor data splitting results. However, this approach increases the clients' workload and experimental costs. After long-term experiments, the inventor discovered that adverse factors affecting library output and data splitting included an adapter producing a secondary structure that does not facilitate amplification (shown in FIG. 1). Therefore, the inventor removed index adapters easily producing the above secondary structures from two dimensions: library output and data splitting in CN113999893B. Furthermore, in the Patent CN113999893B, the inventor prioritized the balance of bases in groups of 4. Grouping of 4-base balance is performed on the above screened index sequences, so as to prevent reduced sequencing quality caused by base imbalance from affecting the correct effective splitting of data. The index products developed in CN113999893B have been verified to have higher advantages of data splitting compared to other index products on the market.

However, as the development and application of related products of the Patent CN113999893B became more widespread, the inventor gradually discovered that, with good balance, combinations of different index sequences also affect the output of data, such that an optimal combination needs to be further taken into consideration during practical application. For example, in the Patent CN113999893B, by prioritizing 4-base balance, strict standards are set further from two perspectives of library output and data splitting. When it is required that the normalized values of library output and data splitting shall meet 85%-115%, basically no client complaints about unbalanced data splitting. However, when it is required that the normalized values of library output and data splitting shall meet 80%-120% (i.e., lowering the screening standards), the library output of part of the adapters is low, the data splitting results are not ideal, and the clients feedback that additional experiments need to be conducted. Since the additional experiments generally involve hybridization capture of 12 libraries together, even if the data from one library is insufficient, all 12 libraries need to be re-sequenced, which is equivalent to a 12-fold amplification.

For example, during whole-exome targeted capture sequencing, the index sequences (which are screened out according to screening indicators that the normalized values of library output and data splitting are all meet 80%-120%) in the prior art are used to arrange 12 samples together for hybridization sequencing, 10 GB of data is arranged for each sample, and after the sequencing data is split, the data output of each sample should generally not be less than 8 GB. If the output of each sample after the sequencing data is split is about to be increased, for example, by 1 GB, 2 GB of data needs to be arranged for the sample. Since the 12 samples should be additionally sequenced together, 24 GB of data needs to be additionally sequenced.

However, utilizing the index sequences (which are screened out according to the screening indicators that the normalized values of library output and data splitting are all meet 85%-115%) of the present disclosure to arrange 12 samples for hybridization sequencing, the data to be additionally sequenced may not exceed 80% of the additionally-sequenced data of the index sequences (which are screened out according to screening indicators that the normalized values of library output and data splitting are all meet 80%-120%) in the prior art.

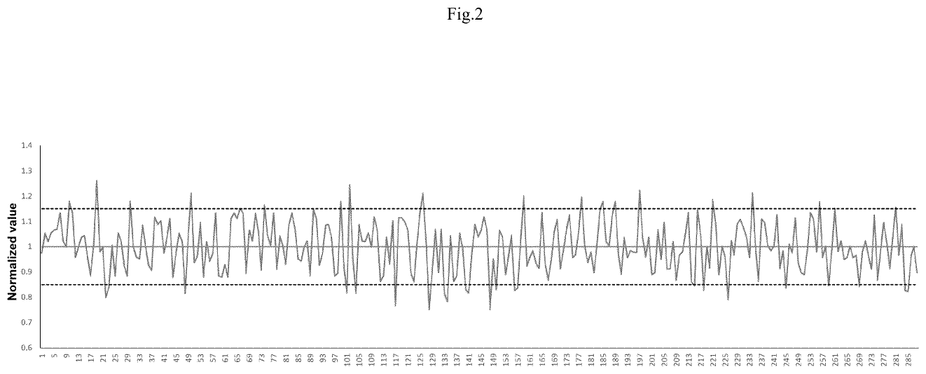

However, if the screening standards are improved, that is, when it is required that the normalized values of library output and data splitting shall meet 85%-115%, more than 50% of combinations among part of the 72 groups of 4-base balanced combinations provided in the Patent CN113999893B (72 groups of combinations formed by P5-end index sequences shown in No. P5-125 to P5-412 in Table 1-1 in Patent CN113999893B and P7-end index sequences shown in No. P7-145 to P7-432 in Table 1-2) cannot be used (shown in FIG. 2). Therefore, in the present disclosure, based on the sequences provided in the Patent CN113999893B, the stable balance of three indicators of library output, whole-genome library sequencing data splitting, and captured library sequencing data splitting is preferably taken into consideration, and then 408 loading combinations that meet a requirement of 8 in a group and ensures each group of four bases being ≥12.5% are arranged in a manner of 8 in a group as far as possible. Compared to the Patent CN113999893B, the present disclosure imposes higher requirements for the stability of library output and data splitting, and their respective advantages and disadvantages are shown in Table 2.

TABLE 2

| | CN113999893B | The present disclosure |
|---|---|---|
| Base balance | Absolute balance of | 8 in a group without |
| relationship | bases in groups of 4 | base absence |
| | (Optimal level) | (Subordinate level) |
| Data splitting | 80-120% | 85-115% |
| standard | (Subordinate level) | (Optimal level) |
| Library output | 80-120% | 85-115% |
| standard | (Subordinate level) | (Optimal level) |
| 0-3 samples | Not available | Not available |
| 4-7 samples | Excellent | Not available |
| Greater than or equal | Excellent | More excellent. |
| to 8 samples | | |

When the throughput is low or the number of samples is small and the sequencing volume of each sample is relatively high (4-7 samples), 4-balance compatibility is an important factor affecting the quality of loading sequencing, but an adapter solution product for 384-group 4-balance compatible with dual sequencing platforms proposed by the Patent CN113999893B can meet the application. However, when the throughput is very high (e.g., during whole-exome capture sequencing, the sequencing volume of each sample is ≥10 GB), and the sample size is very large (e.g., when there are more than 8 samples, especially when there are dozens to hundreds of samples), the solution of the present disclosure is more advantageous. The balance when there are more than 8 samples is no longer an important factor restricting the quality of loading sequencing, and the data balance output after equal mass arrangement loading is a crucial factor. That is, when there are a large number of samples need to be loaded together, the balance of the bases is not the most important factor to consider. The most important factor is the balanced library output of the data. In particular, during hybridization capture, if dual indexes themselves vary greatly, the application quality of the product is seriously affected. Therefore, when there are a large number of adapters need to be loaded, three factors of the balance of library output, the balance of whole-genome library sequencing data splitting, and the balance of captured library sequencing data splitting are used as important standards for screening, facilitating further improvement of the quality of the sequencing data and the balance of output data splitting.

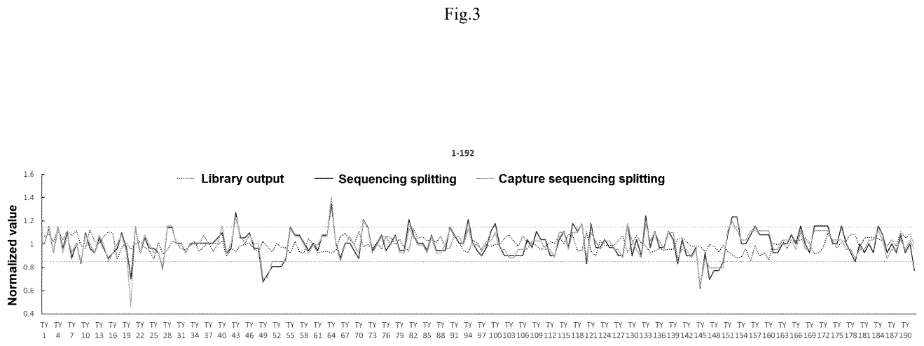

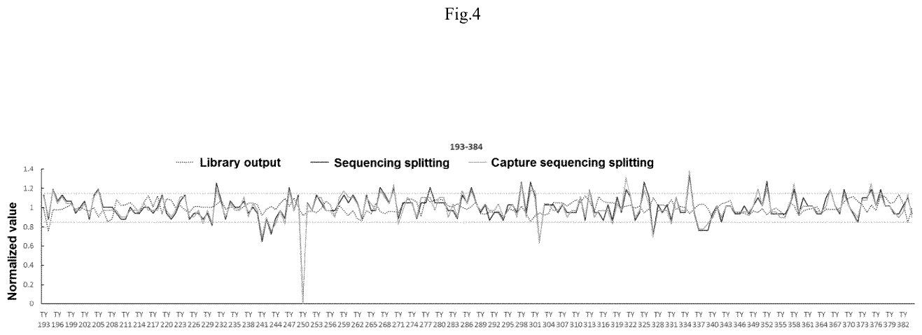

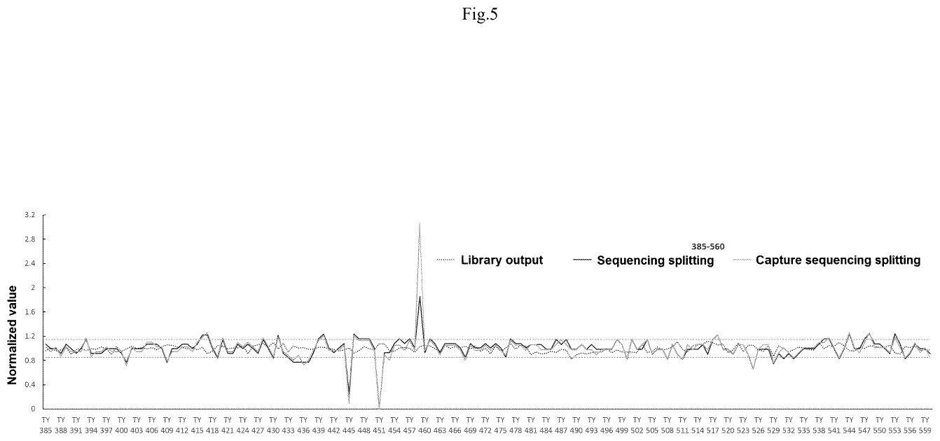

In the present disclosure, 560 groups of unique dual indexes (which may be loaded compatibly with the adapter primers in the Patent CN113999893B) are used for testing, a difference between the index sequences is ≥3 bases, assessment is performed from the three indicators of library output, whole-genome library sequencing data splitting, and captured library sequencing data splitting, and assessment results are shown in FIG. 3, FIG. 4, and FIG. 5. FIG. 3 is a schematic diagram of library output, whole-genome library sequencing data splitting, and captured library sequencing data splitting for 1-192 groups. Correspondingly, FIG. 4 and FIG. 5 are respectively schematic diagrams of library output, whole-genome library sequencing data splitting, and captured library sequencing data splitting for 193-384 groups and 385-560 groups.

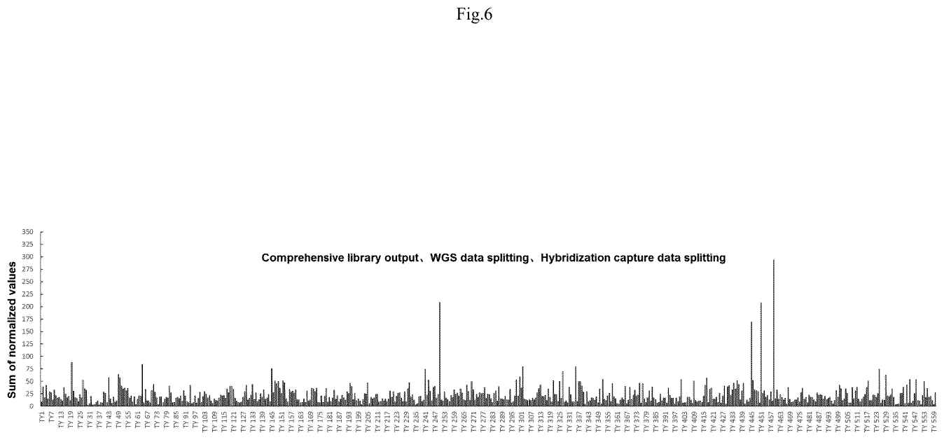

Normalization processing is performed on the above three indicators to obtain rating values of three indicators for each group of the unique dual indexes, and finally, the rating values are added to obtain a rating sum for each group of the unique dual indexes. The process of normalization processing is as follows: utilizing library output as an example, a calculation formula is: rating value for library output=100%*(1−library value/library average). Similarly, whole-genome library sequencing data splitting and captured library sequencing data splitting are also subjected to the normalization processing utilizing the same method. Three rating values are added to obtain a normalized data rating sum, and the smaller the rating sum, the better (shown in FIG. 6).

The screening standard of the present disclosure is that the rate values of the three indicators are all ≤15%, that is, the three indicators are all considered acceptable when being controlled within ±15% (including 15%) of the average. Among the 560 groups of the index sequences, 423 groups meet the screening standard (shown in FIG. 6).

Due to the existence of high-throughput sequencing of a small number of samples or the occurrence of mixed sequencing, the convenience of loading and the balance of the bases should also be taken into consideration. Since a first round of screening is performed in advance based on the above 3 indicators, absolute balance in groups of 8 or 4 cannot be completely achieved. In the present disclosure, when the stable balance of three indicators of library output, whole-genome library sequencing data splitting, and captured library sequencing data splitting is preferably taken into consideration, 423 groups of the index sequences are, in a manner of 8 in a group as far as possible, arranged into 408 loading combinations that meet a requirement of 8 in a group and ensures each group of four bases being ≥12.5%. Loading sequencing is arranged in a manner of 8 in a group. Indexes on P5 and P7 ends are in a fixedly-matched relationship in a one-to-one correspondence manner. For example, when the index on the P5 end is selected from P5-001 in Table 1, the index on the P7 end can only be selected from P7-001 in Table 1. For another example, when the index on the P5 end is selected from P5-002 in Table 1, the index on the P7 end can only be selected from P7-002 in Table 1.

As mentioned in the Background, the rapid development of high-throughput sequencers in the prior art has proposed higher requirements on the types and quality of sequence indexes used during sequencing. The Patent CN113999893B mainly solves the problem of balanced index adapter primers in groups of 4 that are compatible with dual sequencing platforms. The library output and data splitting are relatively stable for less than 8 libraries constructed by the provided sequence indexes, especially 4-7 libraries. However, for more than 8 libraries, the sequence indexes provided in the Patent CN113999893B have the problem of imbalance of library output and data splitting, seriously affecting the quality of loading sequencing.

Therefore, in order to meet the requirements of existing high-throughput sequencing technologies for higher quality of sequencing index adapters, the inventor screens out double-ended fixedly-matched unique index adapter combinations of various fixedly-matched P5 ends and P7 ends based on three indicators, which are library output, whole-genome library sequencing data splitting, and captured library sequencing data splitting, all of which simultaneously meet the standard of differences in normalization less than +15%. Furthermore, in order to take low-throughput sequencing modes or the requirements for high sequencing volumes with small sample sizes into consideration, 51 loading combinations in groups of 8 are further arranged. The sequence indexes of the present disclosure have significant advantages in the application of constructing 8 or more libraries, as evidenced by more balanced data output for each library. Therefore, the inventor proposes a series of protective solutions based on the above problems.

A first typical implementation of the present disclosure provides a method for constructing a DNA library. library construction is performed on a target sample utilizing a primer with 5′ phosphorylation modification or an adapter with 5′ phosphorylation modification, obtaining a linear amplification library with 5′ phosphorylation modification, where the linear amplification library with 5′ phosphorylation modification is a linear library suitable for an Illumina® sequencing platform; and the linear amplification library with 5′ phosphorylation modification is further circularized to obtain a circularized library suitable for an MGI® sequencing platform.

The primer with 5′ phosphorylation modification includes a P5 truncated amplification primer; the adapter with 5′ phosphorylation modification includes a P5 full-length adapter and a P7 full-length adapter.

The P5 truncated amplification primer has the following sequences: 5′-SEQ ID NO: 821-NNNNNNNNNN-SEQ ID NO: 819-3′, wherein a sequence of SEQ ID NO: 821 is AATGATACGGCGACCACCGAGATCTACAC, a sequence of SEQ ID NO: 819 is ACACTCTTTCCCTACACGAC, and N represents a P5-end index sequence; and a 5′ end of the P5 truncated amplification primer is modified through phosphorylation.

A P7 truncated amplification primer has the following sequences: 5′-SEQ ID NO: 823-NNNNNNNNNN-SEQ ID NO: 820-3′, where a sequence of SEQ ID NO: 823 is CAAGCAGAAGACGGCATACGAGAT, a sequence of SEQ ID NO: 820 is GTGACTGGAGTTCAGACGTGT, and N represents a P7-end index sequence.

The P5 full-length adapter has the following sequences: 5′-SEQ ID NO: 821-NNNNNNNNNN-SEQ ID NO: 817-3′, where a sequence of SEQ ID NO: 821 is AATGATACGGCGACCACCGAGATCTACAC, a sequence of SEQ ID NO: 817 is ACACTCTTTCCCTACACGACGCTCTTCCGATC*T, N represents the P5-end index sequence, and * represents thio modification; and a 5′ end of the P5 full-length adapter is modified through phosphorylation.

The P7 full-length adapter has the following sequences: 5′-SEQ ID NO: 818-NNNNNNNNNN-SEQ ID NO: 824-3′, wherein a sequence of SEQ ID NO: 818 is GATCGGAAGAGCACACGTCTGAACTCCAGTCAC, a sequence of SEQ ID NO: 824 is ATCTCGTATGCCGTCTTCTGCTTG, and N represents the P7-end index sequence; and a 5′ end of the P7 full-length adapter is modified through phosphorylation.

The sequences, which are 1 bp from upstream and downstream of the index sequence including the P5-end index sequence or the P7-end index sequence, have at least three edit distances.

A plurality of target samples are provided, and the P5-end index sequences corresponding to the plurality of target samples are selected from P5-end index sequences in any one of loading combinations in Table 1; the P7-end index sequence is selected from fixedly-matched P7-end index sequences corresponding to the P5-end index sequences in Table 1, where the P5-end and the P7-end index sequences all meet the respective number of bases A, T, C, and G in the loading combinations being ≥12.5%; and 8 indexes constitute a group of the loading combinations.

In the above solution, through the linear library constructed by the primer or adapter with 5′ phosphorylation modification, loading sequencing can be directly performed on the Illumina® sequencing platform, or by means of 5′ phosphorylation modification of the library itself, the linear library may also be circularized and prepared into a library suitable for loading sequencing on the MGI® sequencing platform, such that dual sequencing platforms of Illumina® sequencing platform and MGI® sequencing platform are both taken into consideration. In addition, other sequencing platforms compatible with the index sequences of the present disclosure may also use the index sequences of the present disclosure. The method of the present disclosure for constructing a library using the above index sequences is convenient, fast, and compatible with a plurality of platforms. In order to improve the sequencing quality of high-throughput sequencing, in a preferred embodiment of the present disclosure, the P5-end index is selected from any one of index sequence in Table 1, and the P7-end index is selected from a fixedly-matched P7-end index sequence corresponding to the P5-end index sequence in Table 1. Since the index sequences in Table 1 fully considers possible errors and omissions during sequence synthesis, etc., and at least 3 edit distances are provided, mixed sequencing data even when synthetic bases are absent can still be correctly split.

In another preferred embodiment of the present disclosure, when there are a plurality of target samples, the P5-end index sequences corresponding to the plurality of target samples are selected from P5-end index sequences in any one of loading combinations in Table 1; and the P7-end index sequence is selected from fixedly-matched P7-end index sequences corresponding to the P5-end index sequences in Table 1. When the plurality of target samples are sequenced, in order to improve the accuracy of subsequent data splitting, the index sequences in Table 1 are divided into loading combinations, and the respective number of bases A, T, C, and G in each combination is ≥12.5%; and 8 indexes constitute a group of the above loading combinations. When the number of the libraries is 4-7, the present disclosure cannot be used, and the Patent CN113999893B needs to be used to implement sample detection. Depending on whether the primer with 5′ phosphorylation modification is the truncated primer or full-length adapter, there are certain differences in the library construction process.

In a preferred embodiment of the present disclosure, performing library construction on the target sample utilizing the primer with 5′ phosphorylation modification, obtaining the linear amplification library with 5′ phosphorylation modification includes: adapter ligation is performed on a fragment derived from the target sample utilizing truncated adapters shown in SEQ ID NO: 817 and SEQ ID NO: 818, obtaining a fragment with adapters; and the fragment with adapters is amplified utilizing the P5 truncated amplification primer and the P7 truncated amplification primer, obtaining the linear amplification library with 5′ phosphorylation modification. A sequence of SEQ ID NO: 817 is 5′-ACACTCTTTCCCTACACGACGCTCTTCCGATC*T-3′, and represents thio modification; and a sequence of SEQ ID NO: 818 is 5′-GATCGGAAGAGCACACGTCTGAACTCCAGTCAC-3′, and a 5′ end is modified through phosphorylation.

In another preferred embodiment of the present disclosure, performing library construction on the target sample utilizing the adapter with 5′ phosphorylation modification, obtaining the linear amplification library with 5′ phosphorylation modification includes: adapter ligation is performed on a fragment derived from the target sample utilizing the P5 full-length adapter and the P7 full-length adapter, obtaining a fragment with adapters; and the fragment with the adapters is amplified utilizing library amplification primers shown in SEQ ID NO: 822 and SEQ ID NO: 825, obtaining the linear amplification library with 5′ phosphorylation modification. A sequence of SEQ ID NO: 822 is 5′-AATGATACGGCGACCACCGAGAT-3′, and a 5′ end is modified through phosphorylation; and a sequence of SEQ ID NO: 825 is 5′-CAAGCAGAAGACGGCATACGA-3′.

The above two methods for constructing DNA libraries are also suitable for construction of capture libraries. Before circularization in the above step, a step of performing targeted capture on the linear amplification library is added. In a preferred embodiment of the present disclosure, the captured library after targeted capture is amplified utilizing the primer with 5′ phosphorylation modification, obtaining a linear amplified captured library; and the linear amplified captured library is circularized to obtain the circularized library suitable for the MGI® sequencing platform. A second aspect of the present disclosure provides a kit for constructing a DNA library. The kit for constructing a DNA library includes any one of the following combinations: 1) Combination 1: a P5 truncated amplification primer in the above method for constructing a DNA library and a P7 truncated amplification primer in the above method for constructing a DNA library; and 2) Combination 2: a P5 full-length adapter in the above method for constructing a DNA library and a P7 full-length adapter in the above method for constructing a DNA library. The kit for constructing a DNA library includes 408 P5-end index sequences and 408 corresponding fixedly-matched P7-end index sequences, the P5-end index sequences are shown in Table 1 in the method for constructing a DNA library, and the P7-end index sequences are shown in Table 1 in the method for constructing a DNA library. The P5-end index sequence is used in conjunction with a group of 8-base balanced index sequences, and the P7-end index sequence is used according to the fixedly-matched P7-end index sequences corresponding to the P5-end index sequences.

The kit for constructing a DNA library further includes library amplification primers shown in SEQ ID NO: 822 and SEQ ID NO: 825, and/or truncated adapters shown in SEQ ID NO: 817 and 818.

A third aspect of the present disclosure provides an adapter element compatible with dual-sequencing platforms. The adapter element is selected from any one of the following combinations.
1) Combination 1: a P5 truncated amplification primer in the above method for constructing a DNA library and a P7 truncated amplification primer in the above method for constructing a DNA library.
2) Combination 2: a P5 full-length adapter in the above method for constructing a DNA library and a P7 full-length adapter in the above method for constructing a DNA library. The sequences, which are 1 bp from upstream and downstream of the index sequence including the P5-end index sequence or the P7-end index sequence, contain at least three edit distances. The adapter element is an amplification primer composition or an adapter composition. The amplification primer composition includes a combination of the P5 truncated amplification primers and/or the P7 truncated amplification primers; the P5 truncated amplification primers and the P7 truncated amplification primers are each independently of a group or a plurality of groups; each group of the P5 truncated amplification primers includes P5-end index sequences selected from any one of loading combinations in Table 1 in the method for constructing a DNA library; each group of the P7 truncated amplification primers includes fixedly-matched P7-end index sequences corresponding to the P5-end index sequences selected from Table 1 in the Method for constructing a DNA library; the respective number of bases A, T, C, and G in the loading combination is ≥12.5%.

The adapter composition includes a plurality of groups of the P5 full-length adapters and/or a plurality of groups of the P7 full-length adapters; the P5 full-length adapters and the P7 full-length adapters are each independently of a group or a plurality of groups; each group of the P5 full-length adapters includes P5-end index sequences selected from any one of loading combinations in Table 1 in the Method for constructing a DNA library; each group of the P7 full-length adapters includes fixedly-matched P7-end index sequences corresponding to the P5-end index sequences selected from Table 1 in the Method for constructing a DNA library; the respective number of bases A, T, C, and G in the loading combination is ≥12.5%; and 8 indexes constitute a group of the loading combinations.

The beneficial effects of the present disclosure are further described in detail below with reference to specific embodiments.

### Example 1: Assessment of Library Output of Unique Dual Index Libraries (Utilizing Truncated Amplification Primers)

### Step I: Sample Fragmentation

A Covaris™ series DNA ultrasonic disruptor was used to fragment genomic DNA standard samples (Promega®, G1521) to an average fragment size of 250-300 bp.

Step II: End repair & A tailing (NadPrep® DNA universal library construction kit, article number: #1002101, Nanodigmbio (Nanjing) Biotechnology Co., Ltd.)
1. End Repair & A-Tailing Buffer was taken and melted at normal temperature, well mixed, and placed on ice for later use.
2. End Repair & A-Tailing Enzyme was taken and placed on ice for natural melting, well mixed, and subjected to instantaneous centrifugation for later use.
3. According to the following table, a reaction system was prepared in a 0.2 mL PCR tube placed on ice, and the reaction system was shown in Table 3.

TABLE 3

| | |
|---|---|
| Fragmented DNA | 40 μL (10 ng) |
| End Repair & A-Tailing Buffer | 6 μL |
| End Repair & A-Tailing Enzyme | 4 μL |
| Total volume | 50 μL. |

4. Well mixing was performed, and instantaneous centrifugation was performed to cause all reaction liquid to be placed at the bottom of the PCR tube.
5. The following reaction procedures were started on a PCR instrument, a reaction tube was placed in the PCR instrument when a temperature was stabilized to 20° C., and the reaction procedures were shown in Table 4.

TABLE 4

| | |
|---|---|
| 20° C. | 30 min |
| 65° C. | 30 min |
| 10° C. | Hold. |

### Step III: Adapter Ligation

1. NadPrep® Universal Stubby Adapter was formed through annealing of primers SEQ ID NO: 817 and SEQ ID NO: 818; after the two primers were mixed with equal molar, high-temperature incubation was performed for 2 min at 95° C., and then the temperature was slowly cooled to 20° C., so as to form the NadPrep® Universal Stubby Adapter.
SEQ ID NO: 817: ACACTCTTTCCCTACACGACGCTCTTCCGATC*T, and * represented thio modification.
SEQ ID NO: 818: /5Phos/GATCGGAAGAGCACACGTCTGAACTCCAGTCAC, and/5Phos/represented phosphorylation modification; and the two sequences here were the same in CN113999893B, and had the same functions and effects.
2. Ligation Buffer was taken and melted at normal temperature, well mixed, and placed on ice for later use.
3. DNA Ligase was taken and placed on ice for natural melting, well mixed, and subjected to instantaneous centrifugation for later use.
4. The PCR reaction tube in step II was taken out from the PCR instrument and placed on ice, a reaction system was prepared according to the following table, and the reaction system was shown in Table 5.

TABLE 5

| | |
|---|---|
| Reaction product in step II | 50 μL |
| NadPrep ® Universal Stubby Adapter | 2 μL |
| Ligation Buffer | 26 μL |
| DNA Ligase | 2 μL |
| Total volume | 80 μL. |

5. Well mixing was performed, and instantaneous centrifugation was performed to cause all reaction liquid to be placed at the bottom of the PCR tube.
6. The following reaction procedures were started on a PCR instrument, a reaction tube was placed in the PCR instrument when a temperature was stabilized to 20° C., and the reaction procedures were shown in Table 6.

TABLE 6

| | |
|---|---|
| 20° C. | 15 min |
| 4° C. | Hold. |

### Step IV: Purification of Connection Product

1. NadPrep® SP Beads are taken out in advance for vortex mixing, and used after being balanced for 30 min at room temperature.
2. 40 μL of the NadPrep® SP Beads was added to the connection reaction product in step III, well mixed, and incubated for 5-10 min at 25° C.
3. The PCR tube was subjected to instantaneous centrifugation and then placed on a magnetic frame for 5 min until the liquid was completely clear, and supernatant was pipetted utilizing a pipette and then discarded.
4. 150 μL of 80% ethanol was slowly added along a sidewall of the PCR tube, taking care not to disturb magnetic beads, and the PCR tube was allowed to stand for 30 sec; and supernatant was pipetted utilizing a pipette and then discarded.
5. S4 was repeated once.
6. The PCR tube was subjected to instantaneous centrifugation and then placed on the magnetic frame, a 10 μL pipette tip was used to remove a small amount of residual ethanol, taking care not to pipet the magnetic beads.
7. A cap of the PCR tube was opened, and the tube was allowed to stand at room temperature for about 5 min until the ethanol was completely volatilized.
8. The PCR tube was removed out, 20 μL of Nuclease Free Water was added to the PCR tube, and the tube entered step V with the magnetic beads.

### Step V: Amplification of NadPrep® Universal Adapter Ligation Product

2×HiFi PCR Master Mix and NadPrep® Universal Stubby Adapter Primer Mix were taken out and placed on ice for natural melting, well mixed, and subjected to instantaneous centrifugation for later use. The NadPrep® Universal Stubby Adapter Primer Mix was formed by mixing primers SEQ ID NO: 819 and SEQ ID NO: 820 with equal molar.

SEQ ID NO: 819:
ACACTCTTTCCCTACACGAC.
SEQ ID NO: 820:
GTGACTGGAGTTCAGACGTGT.

According to the following table, a reaction system was prepared in a 0.2 mL PCR tube placed on ice (sequentially adding from top to bottom), and the reaction system was shown in Table 7.

TABLE 7

| | |
|---|---|
| Connection product purified in step IV | 20 μL |
| NadPrep ® Universal Stubby Adapter Primer Mix | 5 μL |
| 2 × HiFi PCR Master Mix | 25 μL |
| Total volume | 50 μL. |

The PCR tube was placed in the PCR instrument to start the following procedures, and the reaction procedures were shown in Table 8.

TABLE 8

| | | | |
|---|---|---|---|
| 98° C. | 2 | min | |
| 98° C. | 15 | s | 5 cycles. |
| 60° C. | 30 | s | |
| 72° C. | 30 | s | |
| 72° C. | 2 | min | |

| | |
|---|---|
| 4° C. | Hold |

### Step VI: Purification and Quantification of NadPrep® Universal Stubby Adapter Amplification Product

1. NadPrep® SP Beads are taken out in advance for vortex mixing, and used after being balanced for 30 min at room temperature.
2. 50 μL of NadPrep® SP Beads was added to the amplification product in step V, well mixed, and incubated for 5-10 min at 25° C.
3. The PCR tube was subjected to instantaneous centrifugation and then placed on a magnetic frame for 5 min until the liquid was completely clear, and supernatant was pipetted utilizing a pipette and then discarded.
4. 150 μL of 80% ethanol was slowly added along a sidewall of the PCR tube, taking care not to disturb magnetic beads, and the PCR tube was allowed to stand for 30 sec; and supernatant was pipetted utilizing a pipette and then discarded.
5. S4 was repeated once.
6. The PCR tube was subjected to instantaneous centrifugation and then placed on the magnetic frame, a 10 μL pipette tip was used to remove a small amount of residual ethanol, taking care not to pipet the magnetic beads.
7. A cap of the PCR tube was opened, and the tube was allowed to stand at room temperature for about 5 min until the ethanol was completely volatilized.
8. The PCR tube was removed out, 50 μL of Nuclease Free Water was added to the PCR tube, a pipette was used to suspend the magnetic beads evenly, and incubation was performed for 2 min at 25° C.
9. The PCR tube was subjected to instantaneous centrifugation and then placed on the magnetic frame for 2 min until the liquid was completely clear, and supernatant was transferred to a new 0.2 mL PCR tube utilizing the pipette, taking care not to pipet the magnetic beads.
10. The purified product was quantified utilizing methods such as Qubit or quantitative PCR, etc.
11. The Nuclease Free Water was used to dilute the purified product and a final concentration was 1 ng/μL.

### Step VII: Amplification of NadPrep® Universal UDI-Index Primer Mix

1. 2× HiFi PCR Master Mix and NadPrep® Universal UDI-Index Primer Mix were taken out and placed on ice for natural melting, well mixed, and subjected to instantaneous centrifugation for later use. The NadPrep® Universal UDI-Index Primer Mix was formed by mixing a P5 truncated amplification primer and a P7 truncated amplification primer with equal molar.

The P5 truncated amplification primer had the following sequences: 5′-SEQ ID NO: 821-NNNNNNNNNN-SEQ ID NO: 819-3′, where a sequence of SEQ ID NO: 821 was AATGATACGGCGACCACCGAGATCTACAC, a sequence of SEQ ID NO: 819 was ACACTCTTTCCCTACACGAC, and N represented the index sequence shown in SEQ ID NO:1 at the P5 end.

The P7 truncated amplification primer had the following sequences: 5′-SEQ ID NO: 823-NNNNNNNNNN-SEQ ID NO: 820-3′, where a sequence of SEQ ID NO: 823 was CAAGCAGAAGACGGCATACGAGAT, a sequence of SEQ ID NO: 820 was GTGACTGGAGTTCAGACGTGT, and N represented the index sequence shown in SEQ ID NO: 409 at the P7 end. It was to be noted that, the adapters shown in Table 1 were mixed in a one-to-one relationship, and the horizontal P5 and P7 in the table were assessed in a one-to-one matching manner.

2. According to the following table, a reaction system was prepared in a 0.2 mL PCR tube placed on ice (sequentially adding from top to bottom), and the reaction system was shown in Table 9.

TABLE 9

| | | |
|---|---|---|
| Connection product purified in step VI | 10 | μL |
| NadPrep ® Universal UDI-Index Primer Mix | 2.5 | μL |
| 2 × HiFi PCR Master Mix | 12.5 | μL |
| Total volume | 25 | μL. |

The PCR tube was placed in the PCR instrument to start the following procedures, and the reaction procedures were shown in Table 10.

TABLE 10

| | | | |
|---|---|---|---|
| 98° C. | 2 | min | |
| 98° C. | 15 | s | 8 cycles. |
| 60° C. | 30 | s | |
| 72° C. | 30 | s | |
| 72° C. | 2 | min | |

| | |
|---|---|
| 4° C. | Hold |

Step VIII: Purification and quantification of amplification library
1. NadPrep® SP Beads are taken out in advance for vortex mixing, and used after being balanced for 30 min at room temperature.
2. 25 μL of NadPrep® SP Beads was added to the amplification product in step VII, well mixed, and incubated for 5-10 min at 25° C.
3. The PCR tube was subjected to instantaneous centrifugation and then placed on a magnetic frame for 5 min until the liquid was completely clear, and supernatant was pipetted utilizing a pipette and then discarded.
4. 150 μL of 80% ethanol was slowly added along a sidewall of the PCR tube, taking care not to disturb magnetic beads, and the PCR tube was allowed to stand for 30 sec; and supernatant was pipetted utilizing a pipette and then discarded.
5. S4 was repeated once.
6. The PCR tube was subjected to instantaneous centrifugation and then placed on the magnetic frame, a 10 μL pipette tip was used to remove a small amount of residual ethanol, taking care not to pipet the magnetic beads.
7. A cap of the PCR tube was opened, and the tube was allowed to stand at room temperature for about 5 min until the ethanol was completely volatilized.
8. The PCR tube was removed out, 30 μL of a TE Solution was added to the PCR tube, a pipette was used to suspend the magnetic beads evenly, and incubation was performed for 2 min at 25° C.
9. The PCR tube was subjected to instantaneous centrifugation and then placed on the magnetic frame for 2 min until the liquid was completely clear, and supernatant was transferred to a new 0.2 mL PCR tube utilizing the pipette, taking care not to pipet the magnetic beads.
10. The purified product was quantified utilizing methods such as Qubit or quantitative PCR, etc. Library output assessment was performed on the fixed P5 and P7 unique dual index combinations, and the library output of each group was assessed. As shown in FIG. 3, FIG. 4, and FIG. 5, the selection standard was within +15% of the average as a candidate, and those exceeding this range were excluded. Among the 560 groups, 3 groups were less than 85% of the average, 10 groups were greater than 115%, and 547 groups out of 560 were qualified, with a qualification rate of 97.6%. Since the candidate combinations had already been analyzed at an analytical level, the combinations that were likely to affect amplification efficiency had been screened and screened out, that is, the sequences that were completely complementary to the 3′ end of the index adapter primer with more than 5 bases were filtered in the present disclosure. If there were 7 bases that were completely complementary, as shown in FIG. 1, the library output was about to decrease by 70%.

### Example 2 Assessment of Data Splitting of Equal Ratio of Unique Dual Index Libraries which are Sequenced (Using Truncated Amplification Primers)

Experimental procedures in this Example were the same as Example 1, and this Example was mainly intended to assess whole-genome library sequencing data splitting after the libraries were mixed with equal ratio.

S1: Sequencing after Mixing with Equal Ratio

For the above libraries in Example 1, 5 ng of each library was taken and mixed together, 20 ng of the mixed libraries after mixing with equal ratio was taken to arrange loading sequencing, and each library was arranged for 0.5 GB of data on NovaSeq 6000.

Results were shown in FIG. 3, FIG. 4, and FIG. 5, according to the standard of +15%, among the 560 groups, 50 groups had a failure rate of less than 85%, 62 groups had a failure rate of more than 115%, and 448 groups were qualified, with a qualification rate of 80%. This Example illustrates that, despite the dual index sequences consisting of only 20 bases, the presence of different bases significantly affected sequencing quality, highlighting the necessity of the screening methods described herein.

### Example 3: Assessment of Data Splitting with Different Unique Dual Index Libraries were Captured with Equal Ratio (Using Truncated Amplification Primers)

Forty-eight libraries were randomly selected from the libraries in Example 1, 100 ng of each library was taken for hybridization capture, specific experimental flows were referred to the DNA library hybridization capture (Illumina® sequencing platform) operation guide (Version 2.5), and the input amount for hybridization of each library was 100 ng, and the total input amount for 48 libraries was 4800 ng. 0.5 GB of data was arranged for each library after capture.

Results were shown in FIG. 3, FIG. 4, and FIG. 5, according to the standard of +15%, among the 560 groups, 44 groups had a failure rate of less than 85%, 48 groups had a failure rate of more than 115%, and 468 groups were qualified, with a qualification rate of 83.5%. Captured library sequencing data splitting was highly related to whole-gene library sequencing data splitting.

In the present disclosure, three indicators of library output, whole-genome library sequencing data splitting, and captured library sequencing data splitting of dual index adapter primer combinations after fixed combination were assessed, when the quality was assessed separately, the average difference being less than +15% was used as a standard, index adapter combinations that met the above standard were screened from 560 groups of index adapter combinations. The library output had a high acceptance number, with a qualification rate of 97.6%. The qualification rates for whole-genome library sequencing data splitting and captured library sequencing data splitting were 80% and 83.5%, respectively.

### Example 3: Assessment of Performance of Different Unique Dual Indexes in Full-Length Adapter (Using Full-Length Adapter)

### I. Balance Library Output

The adapter in step III in Example 1 was replaced with a full-length adapter, and the full-length adapter had the following structure.

The P5 full-length adapter had the following sequences: 5′-SEQ ID NO: 821-NNNNNNNNNN-SEQ ID NO: 817-3′, where a sequence of SEQ ID NO: 821 was AATGATACGGCGACCACCGAGATCTACAC, a sequence of SEQ ID NO: 817 was ACACTCTTTCCCTACACGACGCTCTTCCGATC*T, N represented the index sequence shown in SEQ ID NO: 1 at the P5 end, and * represented thio modification; and a 5′ end of the P5 full-length adapter was modified through phosphorylation.

The P7 full-length linker had the following sequences: 5′-SEQ ID NO: 818-NNNNNNNNNN-SEQ ID NO: 824-3′, where a sequence of SEQ ID NO: 818 was GATCGGAAGAGCACACGTCTGAACTCCAGTCAC, a sequence of SEQ ID NO: 824 was ATCTCGTATGCCGTCTTCTGCTTG, and N represented the index sequence shown in SEQ ID NO: 409 at the P7 end. It was to be noted that, the adapters shown in Table 1 were mixed in a one-to-one relationship, the horizontal P5 and P7 in the table were assessed in a one-to-one matching manner, and the 5′ end of the P7 full-length adapter was modified through phosphorylation.

Then, the amplification primers in step V were replaced with sequences shown in SEQ ID NO: 822 and SEQ ID NO: 825.

a sequence of SEQ ID NO: 822 is
5′-AATGATACGGCGACCACCGAGAT-3′, and a 5′ end is modified through phosphorylation;
and
a sequence of SEQ ID NO: 825 is
5′-CAAGCAGAAGACGGCATACGA-3′.

Step VII and step VIII were removed, and others were the same in Example 1.

II. Loading Data Splitting of Library with Equal Ratio

The library in Example 2 was replaced with a library constructed utilizing the full-length adapter in Example 4, and the rest was the same as the Example 2.

III. Loading Data Splitting after Capture of Libraries with Equal Ratio

The library in Example 3 was replaced with a library constructed utilizing the full-length adapter in Example 4, and the rest was the same as the Example 3.

Experimental results showed that, the truncated amplification primer was replaced with the full-length adapter, and then the qualification rates of library output, whole-genome library sequencing data splitting, and captured library sequencing data splitting were similar to those of the truncated amplification primer.

In order to cause the above three indicators to be able to be superposed together, in the present disclosure, normalization processing was performed on the three indicators, three normalized values were added to obtain a sum of three normalized data rating values, and the smaller the value, the better. The screening standard of the present disclosure was that each indicator was controlled within +15% (i.e., the difference between each indicator and the average was less than 15%). Among the 560 groups of the index combinations in Example 1, a total of 423 groups of combinations met the screening standard, with a compliance rate of 75.5%. In the present disclosure, the current requirement for high-throughput sequencers to load a large number of samples at one time was met, such that double-ended unique index adapter combinations were optimized, and the one-to-one relationship between the P5 and P7 index sequences was fixed preferably. Furthermore, the possibility of compatibility with low-throughput loading solutions or situations where a small number of samples generate a large amount of sequencing data were taken into consideration, such that 423 groups of combinations meeting the screening standard were further arranged into groups of 8, with 20 label sequences arranged in a way that no bases were absent, obtaining 51 loading combinations shown in Table 1. Bold fonts and non-bold fonts were used in Table 1 to distinguish different loading combinations in groups of 8.

From the above description, it might be seen that, the above embodiments of the present disclosure implemented the following technical effects. In the present disclosure, the stability (the normalization difference of each indicator was less than +15%) of the three indicators, which were library output, whole-genome library sequencing data splitting, and captured library sequencing data splitting, was used as the screening standard, double-ended fixedly-matched unique index adapter combinations of various fixedly-matched P5 ends and P7 ends were screened out. Furthermore, in order to take low-throughput sequencing modes or the requirements for high sequencing volumes with small sample sizes into consideration, 51 loading combinations in groups of 8 were further arranged (shown in Table 1).

The index combination modes of the present disclosure can better solve the loading problem of the current dual sequencing platforms from Illumina® sequencing platform and MGI® sequencing platform and other sequencing platforms compatible with adapters of the present disclosure, and meet the increasing requirements for the types and quality of sequencing linkers due to the ever-increasing throughput of current high-throughput sequencers. The newly launched adapters introduced stricter control indicators in terms of library output and data splitting (whole-genome library sequencing data splitting and captured library sequencing data splitting), thereby facilitating balanced output control of simultaneous loading of a plurality of samples, thus further facilitating large-scale production.

The above are only the preferred embodiments of the present disclosure and are not intended to limit the present disclosure. For those skilled in the art, the present disclosure may have various modifications and variations. Any modifications, equivalent replacements, improvements and the like made within the spirit and principle of the present disclosure all fall within the scope of protection of the present disclosure.

1. A method for constructing a DNA library, comprising:
performing library construction on a target sample utilizing a primer with 5′ phosphorylation modification or an adapter with 5′ phosphorylation modification, and obtaining a linear amplification library with 5′ phosphorylation modification, wherein the linear amplification library with 5′ phosphorylation modification is configured for sequencing on a platform utilizing bridge amplification and sequencing-by-synthesis chemistry; or
further circularizing the linear amplification library with 5′ phosphorylation modification to obtain a circularized library configured for sequencing on a platform utilizing DNA nanoball generation and combinatorial probe anchor polymerization, wherein
the primer with 5′ phosphorylation modification comprises a P5 truncated amplification primer; the adapter with 5′ phosphorylation modification comprises a P5 full-length adapter and a P7 full-length adapter;
the P5 truncated amplification primer has the following sequences: 5′-SEQ ID NO: 821-NNNNNNNNNN-SEQ ID NO: 819-3′, wherein a sequence of SEQ ID NO: 821 is AATGATACGGCGACCACCGAGATCTACAC, a sequence of SEQ ID NO: 819 is ACACTCTTTCCCTACACGAC, and N represents a P5-end index sequence; a 5′ end of the P5 truncated amplification primer is modified through phosphorylation;
a P7 truncated amplification primer has the following sequences: 5′-SEQ ID NO: 823-NNNNNNNNNN-SEQ ID NO: 820-3′, wherein a sequence of SEQ ID NO: 823 is CAAGCAGAAGACGGCATACGAGAT, a sequence of SEQ ID NO: 820 is GTGACTGGAGTTCAGACGTGT, and N represents a P7-end index sequence;
the P5 full-length adapter has the following sequences: 5′-SEQ ID NO: 821-NNNNNNNNNN-SEQ ID NO: 817-3′, wherein a sequence of SEQ ID NO: 821 is AATGATACGGCGACCACCGAGATCTACAC, a sequence of SEQ ID NO: 817 is ACACTCTTTCCCTACACGACGCTCTTCCGATC*T, N represents the P5-end index sequence, and * represents a thio modification; a 5′ end of the P5 full-length adapter is modified through phosphorylation;
the P7 full-length adapter has the following sequences: 5′-SEQ ID NO: 818-NNNNNNNNNN-SEQ ID NO: 824-3′, wherein a sequence of SEQ ID NO: 818 is GATCGGAAGAGCACACGTCTGAACTCCAGTCAC, a sequence of SEQ ID NO: 824 is ATCTCGTATGCCGTCTTCTGCTTG, and N represents the P7-end index sequence; a 5′ end of the P7 full-length adapter is modified through phosphorylation;
the sequences, which are 1 bp from upstream and downstream of the index sequence comprising the P5-end index sequence or the P7-end index sequence, have at least three edit distances;
≥8 target samples are provided, and the P5-end index sequences corresponding to the plurality of target samples are selected from P5-end index sequences in any one of loading combinations in Table 1; the P7-end index sequence is selected from fixedly-matched P7-end index sequences corresponding to the P5-end index sequences in Table 1, wherein the P5-end index sequences and the P7-end index sequences all meet the respective number of bases A, T, C, and G in the loading combinations being ≥12.5%; and 8 indexes constitute a group of the loading combinations, and Table 1 is as follows:
TABLE 1

| | SEQ ID | p5-end | P5-end | SEQ ID | p7-end | P7-end |
|---|---|---|---|---|---|---|
| Number | NO: | number | sequence | NO: | number | sequence |
| TY491 | 1 | p5-001 | GACCTCGGTT | 409 | p7-001 | ACCTTGTGTT |
| TY324 | 2 | p5-002 | CGGAAGCTGA | 410 | p7-002 | GGAAGTGACC |
| TY025 | 3 | p5-003 | ACGCGTTCAA | 411 | p7-003 | TATCCTCTGG |
| TY266 | 4 | p5-004 | GTTGCAAGTC | 412 | p7-004 | CTAGACCAAT |
| TY124 | 5 | p5-005 | TTCTGGATCG | 413 | p7-005 | AGCAGTACTA |
| TY197 | 6 | p5-006 | GCAGAACATT | 414 | p7-006 | AAGCCATGAG |
| TY370 | 7 | p5-007 | AATTCCTACC | 415 | p7-007 | TCTTAGCCGA |
| TY299 | 8 | p5-008 | TGAAGACCAA | 416 | p7-008 | GCGATAATAC |
| TY438 | 9 | p5-009 | CTTCTGAGTA | 417 | p7-009 | AGAGGCATGG |
| TY252 | 10 | p5-010 | AAGTGCCAGG | 418 | p7-010 | TTCTATCCTC |
| TY340 | 11 | p5-011 | GCAAGTATAC | 419 | p7-011 | TGTCTGGACT |
| TY164 | 12 | p5-012 | TGGCAATGGC | 420 | p7-012 | CAGCGGTGAA |
| TY486 | 13 | p5-013 | TTCGTTACCT | 421 | p7-013 | ACAACTACAG |
| TY344 | 14 | p5-014 | CTTGCGGTAA | 422 | p7-014 | GAGGAAGTAA |
| TY474 | 15 | p5-015 | GGAACACATT | 423 | p7-015 | ATTGTCAGCT |
| TY503 | 16 | p5-016 | ACATAGTCCT | 424 | p7-016 | TCGCCAGATC |
| TY444 | 17 | p5-017 | GAGATCTTGT | 425 | p7-017 | TCGACACAGA |
| TY169 | 18 | p5-018 | CGTGCTAACC | 426 | p7-018 | GAATCCACAG |
| TY391 | 19 | p5-019 | ATAAGGCCTC | 427 | p7-019 | CCTCTTGATT |
| TY182 | 20 | p5-020 | TCGTACGGAA | 428 | p7-020 | ATCTGGAGGT |
| TY082 | 21 | p5-021 | ATTCCACCGA | 429 | p7-021 | GGTGAGCTCA |
| TY092 | 22 | p5-022 | TACGTACTCT | 430 | p7-022 | CTCCAATTCC |
| TY381 | 23 | p5-023 | GGCCTTAGAG | 431 | p7-023 | TGACGTCAAT |
| TY481 | 24 | p5-024 | CCAAGTGAGA | 432 | p7-024 | AAGAATGGAC |
| TY513 | 25 | p5-025 | GGTTGTAACT | 433 | p7-025 | ATTCCACACA |
| TY276 | 26 | p5-026 | CTCCTCGCAA | 434 | p7-026 | TGGTTCTGAA |
| TY085 | 27 | p5-027 | TATGACTGTG | 435 | p7-027 | TACAATGCTC |
| TY200 | 28 | p5-028 | AGAACAGTCC | 436 | p7-028 | GTAGTGGTGC |
| TY103 | 29 | p5-029 | ATGCTGCGGA | 437 | p7-029 | CGATCCATTG |
| TY422 | 30 | p5-030 | GACTCACAGC | 438 | p7-030 | ACAAGACTGT |
| TY076 | 31 | p5-031 | TCTCGGTTAT | 439 | p7-031 | CGCGGTTCAT |
| TY505 | 32 | p5-032 | GGATATACCA | 440 | p7-032 | ATTCGGAACA |
| TY022 | 33 | p5-033 | CGCAGATCCA | 441 | p7-033 | ACACAAGAAC |
| TY421 | 34 | p5-034 | TAGCCTATGG | 442 | p7-034 | GTTACCAGCG |
| TY037 | 35 | p5-035 | ACTGACCATA | 443 | p7-035 | GAGTTGGCTC |
| TY349 | 36 | p5-036 | GGCTACTGCT | 444 | p7-036 | TTCGGTTAGT |
| TY177 | 37 | p5-037 | CTAGTGTACC | 445 | p7-037 | CGAGCCTCAA |
| TY242 | 38 | p5-038 | TCTTCGGAGG | 446 | p7-038 | AGCAGGCTCA |
| TY502 | 39 | p5-039 | TTGACCAGAC | 447 | p7-039 | CAGTATAGAG |
| TY188 | 40 | p5-040 | CAACTTGCAA | 448 | p7-040 | GGTTCACCGT |
| TY396 | 41 | p5-041 | GGTTGATCGA | 449 | p7-041 | TGTTCGTTCG |
| TY168 | 42 | p5-042 | ATAATGGTCG | 450 | p7-042 | ACAGTCCATT |
| TY466 | 43 | p5-043 | GACAACCATT | 451 | p7-043 | CTGTGAAGAT |
| TY017 | 44 | p5-044 | ACGGTCACAC | 452 | p7-044 | GCTATTCTGA |
| TY105 | 45 | p5-045 | CGGCCAATGT | 453 | p7-045 | GACGAGGAAT |
| TY130 | 46 | p5-046 | TGGAATTGTC | 454 | p7-046 | TTGACAGCGC |
| TY309 | 47 | p5-047 | CAACGAGTCG | 455 | p7-047 | AGCCGATGTC |
| TY056 | 48 | p5-048 | ACCTCCACAA | 456 | p7-048 | TAACACAGTG |
| TY251 | 49 | p5-049 | GGAATCTCCT | 457 | p7-049 | ACTAGCGGAC |
| TY480 | 50 | p5-050 | AAGCGAGGAA | 458 | p7-050 | TTAGCAACGT |
| TY132 | 51 | p5-051 | CATTAGATCG | 459 | p7-051 | CACAATATCG |
| TY475 | 52 | p5-052 | TTCTTCTAGC | 460 | p7-052 | ACCTGGTATA |
| TY399 | 53 | p5-053 | ATTGCTCCTA | 461 | p7-053 | ACACTTCTTC |
| TY222 | 54 | p5-054 | GCGATAATCT | 462 | p7-054 | TGGTCAGCAG |
| TY295 | 55 | p5-055 | TGTCGTAGTG | 463 | p7-055 | GTATTCCAGA |
| TY523 | 56 | p5-056 | AAGTCCGAAC | 464 | p7-056 | CGTGACTGCT |
| TY180 | 57 | p5-057 | CAAGTCACAA | 465 | p7-057 | AAGCCTCCAT |
| TY174 | 58 | p5-058 | GCCTCATGGT | 466 | p7-058 | ATCTGAATGC |
| TY429 | 59 | p5-059 | TGGCGTCTGT | 467 | p7-059 | GGTAACAGAG |
| TY265 | 60 | p5-060 | ACTCAGGACG | 468 | p7-060 | CCGACTTCCA |
| TY282 | 61 | p5-061 | TAGAGAGGTC | 469 | p7-061 | TCAGTGGAAC |
| TY482 | 62 | p5-062 | GTCTCGCTTC | 470 | p7-062 | TGTCTCTGTT |
| TY109 | 63 | p5-063 | CTTAGCTCAA | 471 | p7-063 | AATTACCGGA |
| TY277 | 64 | p5-064 | GCACAGAACT | 472 | p7-064 | GTCATAATCG |
| TY394 | 65 | p5-065 | CGTTGCATCT | 473 | p7-065 | TTAAGCTGGA |
| TY424 | 66 | p5-066 | ATGAATGCGA | 474 | p7-066 | TATGCTGCAC |
| TY112 | 67 | p5-067 | ACACTATGTG | 475 | p7-067 | CCGATAACTG |
| TY061 | 68 | p5-068 | TGCTAGAGCC | 476 | p7-068 | GGCCTGCATT |
| TY387 | 69 | p5-069 | CAGCTACCAC | 477 | p7-069 | AAGTACGTAC |
| TY118 | 70 | p5-070 | GTAGCCAATA | 478 | p7-070 | TCCTTATGCC |
| TY501 | 71 | p5-071 | GATGGATTGT | 479 | p7-071 | CTACGGCCTA |
| TY400 | 72 | p5-072 | TCCATGGAGC | 480 | p7-072 | ACCTATGAGA |
| TY291 | 73 | p5-073 | GAATAAGCCA | 481 | p7-073 | AGCCGCGTAT |
| TY011 | 74 | p5-074 | AGTATGCTGA | 482 | p7-074 | TTGTTGTCTG |
| TY013 | 75 | p5-075 | TTCTACTGTC | 483 | p7-075 | CCAGAACGGA |
| TY001 | 76 | p5-076 | CCGGAATATT | 484 | p7-076 | GGAACGATCC |
| TY060 | 77 | p5-077 | GAGCGGTAAG | 485 | p7-077 | CATCCTGCAG |
| TY303 | 78 | p5-078 | GTAGCTAGGC | 486 | p7-078 | AACGCTTAGA |
| TY035 | 79 | p5-079 | CACATTACGT | 487 | p7-079 | TTGATTGACC |
| TY134 | 80 | p5-080 | TCCTTCGTTA | 488 | p7-080 | GCCTTACGTT |
| TY055 | 81 | p5-081 | CGTGGTGCTA | 489 | p7-081 | ATATCAGTCC |
| TY048 | 82 | p5-082 | ACGACAAGAC | 490 | p7-082 | TCCGACAGGA |
| TY077 | 83 | p5-083 | GAATACCTCG | 491 | p7-083 | AAGATTGCTG |
| TY245 | 84 | p5-084 | TGGCTGGTGT | 492 | p7-084 | TATCGGTACG |
| TY258 | 85 | p5-085 | CTTCAACGAC | 493 | p7-085 | CGAGAACTAT |
| TY392 | 86 | p5-086 | TCCACGTAGG | 494 | p7-086 | GACAGAAGCG |
| TY450 | 87 | p5-087 | GTCAGGCACA | 495 | p7-087 | GCATTGCCTC |
| TY373 | 88 | p5-088 | GGATTCACTT | 496 | p7-088 | AGTACTGAAG |
| TY343 | 89 | p5-089 | AAGCTCCATA | 497 | p7-089 | ACATCGCCTA |
| TY256 | 90 | p5-090 | GTCAATATCC | 498 | p7-090 | TTCCAAGTCG |
| TY545 | 91 | p5-091 | CTATCGGCCT | 499 | p7-091 | AGGACTAATC |
| TY005 | 92 | p5-092 | GGTCAAGGTG | 500 | p7-092 | CCTGGATGGA |
| TY506 | 93 | p5-093 | CAGGTGCAGT | 501 | p7-093 | GATAGCTGAT |
| TY154 | 94 | p5-094 | TCACGACTAA | 502 | p7-094 | CGCGTTAGGT |
| TY407 | 95 | p5-095 | ATCGGTTGAA | 503 | p7-095 | TACCAGCTAT |
| TY354 | 96 | p5-096 | ACTTCATTGC | 504 | p7-096 | CTCTACGTCG |
| TY047 | 97 | p5-097 | GGCGAGACAA | 505 | p7-097 | TTATGAGGCC |
| TY041 | 98 | p5-098 | CTGCCTTGTG | 506 | p7-098 | ACCAAGCAGG |
| TY551 | 99 | p5-099 | ACCTTCAAGT | 507 | p7-099 | GTGGACAGAA |
| TY111 | 100 | p5-100 | ATAACGCGCA | 508 | p7-100 | CGACCTTCTG |
| TY075 | 101 | p5-101 | TGTGGAGTCC | 509 | p7-101 | TTACACTCGT |
| TY552 | 102 | p5-102 | GAACTTCGGA | 510 | p7-102 | TACGTTGTTC |
| TY101 | 103 | p5-103 | CATAACTTCG | 511 | p7-103 | AATTCAGGCA |
| TY224 | 104 | p5-104 | TCCTGGCATT | 512 | p7-104 | TCGATGCTAA |
| TY404 | 105 | p5-105 | CGATGCACGA | 513 | p7-105 | AATTCTCACG |
| TY098 | 106 | p5-106 | ACTATACACC | 514 | p7-106 | TCGCAGTGAC |
| TY087 | 107 | p5-107 | CGACCTGTGT | 515 | p7-107 | GGCAGAGTTA |
| TY538 | 108 | p5-108 | GACAGTTGTA | 516 | p7-108 | TTCTTAACGG |
| TY131 | 109 | p5-109 | TTGGCTGCCT | 517 | p7-109 | CAATGCCAAT |
| TY031 | 110 | p5-110 | CACAACCGAG | 518 | p7-110 | ACAGGAGCCA |
| TY008 | 111 | p5-111 | ACTGAGTAAC | 519 | p7-111 | CGTTCTACTT |
| TY360 | 112 | p5-112 | GTCCTAGTAA | 520 | p7-112 | ATGCAGATTC |
| TY199 | 113 | p5-113 | GATCTCGATA | 521 | p7-113 | TCCATGGCTG |
| TY345 | 114 | p5-114 | TGATCTTCGC | 522 | p7-114 | GTACCAAGAA |
| TY193 | 115 | p5-115 | TCTCAGCGCT | 523 | p7-115 | AGTGGTCAAT |
| TY346 | 116 | p5-116 | ACGAGAGTGG | 524 | p7-116 | CGTTACGTGG |
| TY323 | 117 | p5-117 | CGCGTCAGAA | 525 | p7-117 | TACTTACGCA |
| TY389 | 118 | p5-118 | GTCTCTGGAT | 526 | p7-118 | TGGCGTTAAC |
| TY382 | 119 | p5-119 | CAGTGGAATC | 527 | p7-119 | GAAGAGTACA |
| TY186 | 120 | p5-120 | ACTTGTCTGT | 528 | p7-120 | ATGACCATTC |
| TY415 | 121 | p5-121 | AGCTGTACAA | 529 | p7-121 | TGGTAGGAAG |
| TY057 | 122 | p5-122 | CTAGCATGAT | 530 | p7-122 | GTAGTTCGGA |
| TY059 | 123 | p5-123 | TAGATTCTCC | 531 | p7-123 | CCTAGTACAT |
| TY541 | 124 | p5-124 | ACATACGATG | 532 | p7-124 | CACTGCGTCT |
| TY218 | 125 | p5-125 | TGTCGGTGGA | 533 | p7-125 | TAACACCTTC |
| TY063 | 126 | p5-126 | CTGGATGCCA | 534 | p7-126 | ACGGCATTCT |
| TY442 | 127 | p5-127 | GCTAGACAGG | 535 | p7-127 | CTACTGTCTC |
| TY427 | 128 | p5-128 | AGCATGTTCC | 536 | p7-128 | AGGCGTAAGA |
| TY198 | 129 | p5-129 | TACGGAAGAA | 537 | p7-129 | TCATCCAGGA |
| TY465 | 130 | p5-130 | CGACTGACTC | 538 | p7-130 | AGCATTCTCG |
| TY493 | 131 | p5-131 | GCTTGCTTAT | 539 | p7-131 | CAACGTCCTC |
| TY136 | 132 | p5-132 | AGGCCACAGA | 540 | p7-132 | AGTGTGGAAT |
| TY183 | 133 | p5-133 | GACAATGAGT | 541 | p7-133 | GCGCAAGTCA |
| TY215 | 134 | p5-134 | ATCTCCGGTG | 542 | p7-134 | CTCAGCTCCA |
| TY359 | 135 | p5-135 | TCTACTCGCG | 543 | p7-135 | AACACATGGT |
| TY072 | 136 | p5-136 | AGAGTCTCTT | 544 | p7-136 | GATGCGAGAC |
| TY522 | 137 | p5-137 | CACTGCAATT | 545 | p7-137 | AATCGACCGA |
| TY520 | 138 | p5-138 | ACTGACTTGA | 546 | p7-138 | TCGGAGTTCC |
| TY024 | 139 | p5-139 | TAACCGGACC | 547 | p7-139 | CGATGGAGTG |
| TY240 | 140 | p5-140 | TCCATTCCAA | 548 | p7-140 | GTCTCAGTAG |
| TY470 | 141 | p5-141 | TTGTGAGGCG | 549 | p7-141 | TACACTTGCA |
| TY332 | 142 | p5-142 | GGAGAGTTAT | 550 | p7-142 | GTAATCGACG |
| TY398 | 143 | p5-143 | AAGGATAGCC | 551 | p7-143 | AGTGAGACGT |
| TY226 | 144 | p5-144 | TGTCCGGCTA | 552 | p7-144 | CACCTCCTAC |
| TY159 | 145 | p5-145 | CTGCTCGTCT | 553 | p7-145 | ACTTCCGTCC |
| TY137 | 146 | p5-146 | GCTAGTCGAA | 554 | p7-146 | AGCCAGTGGA |
| TY156 | 147 | p5-147 | TGAGAGTCGC | 555 | p7-147 | CAGTGATCGG |
| TY496 | 148 | p5-148 | AGATAACCTG | 556 | p7-148 | GTAACTAACC |
| TY498 | 149 | p5-149 | TTCGCGGAGT | 557 | p7-149 | TGTGCCTTAT |
| TY073 | 150 | p5-150 | CAGTATAGGA | 558 | p7-150 | TGAATACGTC |
| TY211 | 151 | p5-151 | TAGAGCATTC | 559 | p7-151 | GTCGGTAAGG |
| TY239 | 152 | p5-152 | ATCCTAACCT | 560 | p7-152 | CAAGTGAGTT |
| TY311 | 153 | p5-153 | GTTAAGTGGT | 561 | p7-153 | TAGGCCGATA |
| TY558 | 154 | p5-154 | CGGCTCCTTA | 562 | p7-154 | ACACTGTAGT |
| TY116 | 155 | p5-155 | TACTGAATCC | 563 | p7-155 | TGCGGTATTA |
| TY423 | 156 | p5-156 | ACAGCAACAA | 564 | p7-156 | TTGTAGTACG |
| TY220 | 157 | p5-157 | AGGAGTTAAC | 565 | p7-157 | AGTACAAGTC |
| TY326 | 158 | p5-158 | TAACAGGCTA | 566 | p7-158 | CTGTATCCAT |
| TY178 | 159 | p5-159 | CCTTGTCAGG | 567 | p7-159 | GACATCATTC |
| TY328 | 160 | p5-160 | TTGGTATGCT | 568 | p7-160 | GTTCTAAGAG |
| TY206 | 161 | p5-161 | AACGTAAGCA | 569 | p7-161 | AAGTCGAGAG |
| TY385 | 162 | p5-162 | CCACAGCTGT | 570 | p7-162 | TCCGGACTCA |
| TY067 | 163 | p5-163 | TATAGCGAAC | 571 | p7-163 | CTTAACACCT |
| TY210 | 164 | p5-164 | GGTCCTTCAC | 572 | p7-164 | GGTTATCTTC |
| TY355 | 165 | p5-165 | TTGCGAGATA | 573 | p7-165 | TGAGTAGATC |
| TY322 | 166 | p5-166 | GAATCCGACT | 574 | p7-166 | ATGCGGTGGT |
| TY213 | 167 | p5-167 | CTGATTCTAG | 575 | p7-167 | TAAGTTGCCT |
| TY160 | 168 | p5-168 | ACGGCGATTG | 576 | p7-168 | ACACACCATG |
| TY253 | 169 | p5-169 | TGACGGAGTA | 577 | p7-169 | AAGTGGTAGG |
| TY559 | 170 | p5-170 | ATTGATGAGC | 578 | p7-170 | TTGGACGGAC |
| TY091 | 171 | p5-171 | GGCTAGCCAT | 579 | p7-171 | GGACTTCGCT |
| TY093 | 172 | p5-172 | TATGCCTTCG | 580 | p7-172 | GATACAGCTA |
| TY549 | 173 | p5-173 | CCGATCCATT | 581 | p7-173 | CACACCGCAT |
| TY388 | 174 | p5-174 | GGTGTATAGA | 582 | p7-174 | CCTCCGATCT |
| TY196 | 175 | p5-175 | CTGTCTGCAC | 583 | p7-175 | AGTTGTCCTA |
| TY032 | 176 | p5-176 | TCACCAATCA | 584 | p7-176 | GTAGGCAATG |
| TY083 | 177 | p5-177 | TGAGAACGGT | 585 | p7-177 | TGACAACTCT |
| TY078 | 178 | p5-178 | GTCCTGAACA | 586 | p7-178 | ACGTGCAAGC |
| TY320 | 179 | p5-179 | ACGAGTTGCC | 587 | p7-179 | TATATCGGAG |
| TY432 | 180 | p5-180 | CATTAAGCTG | 588 | p7-180 | AATACTTCCG |
| TY403 | 181 | p5-181 | CTGCACCTAC | 589 | p7-181 | GTGGTATCAA |
| TY079 | 182 | p5-182 | CGCTCTGATG | 590 | p7-182 | CTCACAGATA |
| TY305 | 183 | p5-183 | GCTGACTATT | 591 | p7-183 | CGACTGATGT |
| TY142 | 184 | p5-184 | TACTTGGCTA | 592 | p7-184 | GGCTCTTGTC |
| TY536 | 185 | p5-185 | CATCAGCCAA | 593 | p7-185 | AACCTACAAC |
| TY296 | 186 | p5-186 | GCCTTGTATT | 594 | p7-186 | TGTTATTGCG |
| TY007 | 187 | p5-187 | TTGACTATCG | 595 | p7-187 | TACACGATCA |
| TY329 | 188 | p5-188 | AATTGTAGGC | 596 | p7-188 | ACTGACGCGT |
| TY306 | 189 | p5-189 | AGGTCCGTAG | 597 | p7-189 | GTATGCGGTC |
| TY225 | 190 | p5-190 | CAAGAACACC | 598 | p7-190 | CCTGACTACG |
| TY454 | 191 | p5-191 | CCTAGATGTA | 599 | p7-191 | CGGATACAGG |
| TY107 | 192 | p5-192 | TGCTTCGAAT | 600 | p7-192 | ATACGTAGGA |
| TY515 | 193 | p5-193 | AAGCTTGGAT | 601 | p7-193 | TGTATCAGAC |
| TY084 | 194 | p5-194 | TCCTGGTTCC | 602 | p7-194 | CTGCCTTCCT |
| TY126 | 195 | p5-195 | GCTATCAAGA | 603 | p7-195 | ACCTTCCAGG |
| TY066 | 196 | p5-196 | GTCGGACTGT | 604 | p7-196 | GAAGGATTGA |
| TY517 | 197 | p5-197 | CCATAACCTT | 605 | p7-197 | ATAAGGCTTG |
| TY113 | 198 | p5-198 | AGTACTCCGC | 606 | p7-198 | GATCAGGCCT |
| TY336 | 199 | p5-199 | TTGGCGGTTG | 607 | p7-199 | GAATGAAGAC |
| TY069 | 200 | p5-200 | AGTTATAGCG | 608 | p7-200 | ACGCTTGATG |
| TY146 | 201 | p5-201 | TGACGCGGAT | 609 | p7-201 | ATCATCACGT |
| TY089 | 202 | p5-202 | CAGAATAACC | 610 | p7-202 | CCAGGTTGAC |
| TY074 | 203 | p5-203 | ATGGCACGGA | 611 | p7-203 | GATCAGTTCA |
| TY268 | 204 | p5-204 | GGCAGCTTAA | 612 | p7-204 | AGGTGACATC |
| TY114 | 205 | p5-205 | CCTGTTGTGT | 613 | p7-205 | TTCTCGGAAG |
| TY353 | 206 | p5-206 | CACTCGACCA | 614 | p7-206 | GCACCACCAA |
| TY104 | 207 | p5-207 | ACACGTCGTG | 615 | p7-207 | TTGATGGCGG |
| TY418 | 208 | p5-208 | GGTATGCACC | 616 | p7-208 | GGACATTGTT |
| TY519 | 209 | p5-209 | TTGAACCGCT | 617 | p7-209 | ACAGCAGATG |
| TY356 | 210 | p5-210 | CGAACTAAGC | 618 | p7-210 | GGCCTTAGAA |
| TY405 | 211 | p5-211 | GGTTAAGCTT | 619 | p7-211 | TTGAGGTAAC |
| TY556 | 212 | p5-212 | GGCGGATGAA | 620 | p7-212 | AGATAACGCT |
| TY151 | 213 | p5-213 | TTGCTGATTG | 621 | p7-213 | TGTTGCATGC |
| TY463 | 214 | p5-214 | CCTCATTAGA | 622 | p7-214 | CAGCACACCT |
| TY361 | 215 | p5-215 | AAGGCGACCA | 623 | p7-215 | GAGAGGTCCA |
| TY464 | 216 | p5-216 | ATCTGTCCGC | 624 | p7-216 | ATGGTTGTGG |
| TY341 | 217 | p5-217 | CGGTAATGAT | 625 | p7-217 | TTCACCTGCT |
| TY284 | 218 | p5-218 | CTCGTTGCGA | 626 | p7-218 | AGATGTCAGA |
| TY294 | 219 | p5-219 | GCACACATGA | 627 | p7-219 | TAGGTGGCTG |
| TY308 | 220 | p5-220 | TACAATCCTC | 628 | p7-220 | GCTAAGTTAC |
| TY016 | 221 | p5-221 | TCTCCGTACC | 629 | p7-221 | CGCTCGAGTT |
| TY297 | 222 | p5-222 | AACGTCTAAG | 630 | p7-222 | CCTCGAATGA |
| TY386 | 223 | p5-223 | AGTGGCGTCT | 631 | p7-223 | ATAATCTCGG |
| TY397 | 224 | p5-224 | GTCTTAGGTT | 632 | p7-224 | TAGAACGAAC |
| TY546 | 225 | p5-225 | AATAGCTGCA | 633 | p7-225 | TCACTAACGA |
| TY018 | 226 | p5-226 | GTCTCAGAAG | 634 | p7-226 | ATCACTTGAC |
| TY201 | 227 | p5-227 | TCAGTGCTCC | 635 | p7-227 | GGTGACCAGT |
| TY202 | 228 | p5-228 | CGTCATACTT | 636 | p7-228 | GATCGTATTC |
| TY122 | 229 | p5-229 | TCGGATTGGC | 637 | p7-229 | TTGTCTGATG |
| TY528 | 230 | p5-230 | AATGGAGCAA | 638 | p7-230 | CATACGGACC |
| TY097 | 231 | p5-231 | CGGTTCATGT | 639 | p7-231 | GCCGGTCTAT |
| TY530 | 232 | p5-232 | GCCAGTTATT | 640 | p7-232 | AGAATACCGA |
| TY033 | 233 | p5-233 | CTGCGTTACA | 641 | p7-233 | AGTAAGATGG |
| TY469 | 234 | p5-234 | TGAGTAGCAT | 642 | p7-234 | TGAGGACCAA |
| TY166 | 235 | p5-235 | GACTCGTTGA | 643 | p7-235 | CTGTAATGTC |
| TY221 | 236 | p5-236 | CTACATCCGG | 644 | p7-236 | GACCTTGACT |
| TY207 | 237 | p5-237 | GATACCGGTC | 645 | p7-237 | ACCTACTCTA |
| TY257 | 238 | p5-238 | ACACCTAATG | 646 | p7-238 | CCAACGTATA |
| TY304 | 239 | p5-239 | ACCTTGTGCT | 647 | p7-239 | TTAGTTCACG |
| TY262 | 240 | p5-240 | TAGGTAGTAC | 648 | p7-240 | TGTTCCGAGC |
| TY472 | 241 | p5-241 | CACCGTCGAA | 649 | p7-241 | ATGTTGACGT |
| TY290 | 242 | p5-242 | TTGAAGGTTC | 650 | p7-242 | GCAAGATAAC |
| TY214 | 243 | p5-243 | GCCTTAACCA | 651 | p7-243 | TAGGCAGGAG |
| TY102 | 244 | p5-244 | TAAGCGTATC | 652 | p7-244 | CCTCATTCTT |
| TY283 | 245 | p5-245 | GATATGAAGG | 653 | p7-245 | AGCAACCGCA |
| TY045 | 246 | p5-246 | AGTGTCCATT | 654 | p7-246 | GATGTCTATC |
| TY509 | 247 | p5-247 | CCTGAACTGT | 655 | p7-247 | TGAACGCTTA |
| TY014 | 248 | p5-248 | CGATGTGCAA | 656 | p7-248 | TAGCGCGAAG |
| TY127 | 249 | p5-249 | CGAGACCAGT | 657 | p7-249 | AGTCGAAGCC |
| TY090 | 250 | p5-250 | ATTAGAGGAC | 658 | p7-250 | TCGTCGCCAA |
| TY456 | 251 | p5-251 | TACCTGACGC | 659 | p7-251 | ATCGCTGTCT |
| TY333 | 252 | p5-252 | CCGTCATCAA | 660 | p7-252 | TGCAACCATT |
| TY286 | 253 | p5-253 | GGACTCATAT | 661 | p7-253 | CAGGAGTTGG |
| TY507 | 254 | p5-254 | ATTCTTGGTG | 662 | p7-254 | TGAGTCTCGC |
| TY281 | 255 | p5-255 | AACAACTCCA | 663 | p7-255 | GTTACAACAC |
| TY144 | 256 | p5-256 | CTTGCACATT | 664 | p7-256 | GCAATAGATG |
| TY115 | 257 | p5-257 | TGCTTGGTCA | 665 | p7-257 | AAGAGCCTGA |
| TY471 | 258 | p5-258 | GCTCATTCAT | 666 | p7-258 | GAACAAGCCG |
| TY557 | 259 | p5-259 | CTCACAACTT | 667 | p7-259 | CGCACTTATT |
| TY162 | 260 | p5-260 | TCAGGCCATG | 668 | p7-260 | TGCATGACGC |
| TY175 | 261 | p5-261 | TACCATTGGA | 669 | p7-261 | GCTGAAGGAA |
| TY125 | 262 | p5-262 | AAGTTAGCTC | 670 | p7-262 | GTTGACGTTA |
| TY510 | 263 | p5-263 | GTTAGGATAC | 671 | p7-263 | CTCTATTGAG |
| TY260 | 264 | p5-264 | AGGAGCCACA | 672 | p7-264 | AAGTGCCGTC |
| TY307 | 265 | p5-265 | TGACGCACAA | 673 | p7-265 | ACGACAACCA |
| TY425 | 266 | p5-266 | CCTTACGATT | 674 | p7-266 | CTATTGCTAC |
| TY378 | 267 | p5-267 | ATTGTACTCC | 675 | p7-267 | TCGTTCTCGG |
| TY263 | 268 | p5-268 | TAGGACTCGA | 676 | p7-268 | GATGATAGGT |
| TY462 | 269 | p5-269 | TCCAGTTGCG | 677 | p7-269 | TACAAGGAGA |
| TY410 | 270 | p5-270 | GGAATGAACT | 678 | p7-270 | GTCCGCAGTT |
| TY128 | 271 | p5-271 | GAACCAAGAA | 679 | p7-271 | AGAGTATGCT |
| TY319 | 272 | p5-272 | CTCTTCCATT | 680 | p7-272 | CGACCGGTTA |
| TY099 | 273 | p5-273 | GGATACGCAA | 681 | p7-273 | ATATGGACGA |
| TY504 | 274 | p5-274 | AACCGTCATT | 682 | p7-274 | AGAATTGTCC |
| TY312 | 275 | p5-275 | TCGTCTTGCC | 683 | p7-275 | TATGTTCCAG |
| TY426 | 276 | p5-276 | AGAGTGCGCA | 684 | p7-276 | TGCCAATGTT |
| TY534 | 277 | p5-277 | GTCTTAATGG | 685 | p7-277 | ATGTCCGTGA |
| TY411 | 278 | p5-278 | CTTACCTTCT | 686 | p7-278 | CCGGTCACTA |
| TY026 | 279 | p5-279 | GAGTTAGAAG | 687 | p7-279 | GCCAGTTAAT |
| TY412 | 280 | p5-280 | CAACGACCTA | 688 | p7-280 | AATACAGACG |
| TY155 | 281 | p5-281 | GGTATTGGAA | 689 | p7-281 | AGCAATCAAC |
| TY514 | 282 | p5-282 | CAAGCGAATT | 690 | p7-282 | TATCGCGCGT |
| TY288 | 283 | p5-283 | TGGTGCCACT | 691 | p7-283 | CCTTAATTCG |
| TY255 | 284 | p5-284 | CTTGTGTTGA | 692 | p7-284 | GAAGTTAGCA |
| TY488 | 285 | p5-285 | ATGCAACGTT | 693 | p7-285 | TTGGCCTAGG |
| TY379 | 286 | p5-286 | TCGCATGCAC | 694 | p7-286 | TGTTAGGTAC |
| TY264 | 287 | p5-287 | TTCAGTGTGG | 695 | p7-287 | TTCCTTGTTG |
| TY038 | 288 | p5-288 | GAATGATCAC | 696 | p7-288 | ACTCCACGTA |
| TY272 | 289 | p5-289 | TTGAGACTGA | 697 | p7-289 | ATTCAGAGTG |
| TY106 | 290 | p5-290 | GCTTATGTCT | 698 | p7-290 | TCGACATTGC |
| TY227 | 291 | p5-291 | GAAGCCAGGT | 699 | p7-291 | GGATGCTCGT |
| TY495 | 292 | p5-292 | TACCAGCCAC | 700 | p7-292 | CAACTAGGAA |
| TY357 | 293 | p5-293 | CGAATCTGTT | 701 | p7-293 | CCTGTTCAGT |
| TY010 | 294 | p5-294 | ATAGCACACT | 702 | p7-294 | AACGTCTACC |
| TY368 | 295 | p5-295 | TTGCTAGCAG | 703 | p7-295 | TTGAGCATAC |
| TY279 | 296 | p5-296 | CGGTCTTATT | 704 | p7-296 | AACTGTGCTT |
| TY223 | 297 | p5-297 | ATGTTCGCAA | 705 | p7-297 | ATTGGCTGAA |
| TY161 | 298 | p5-298 | CATGACAACC | 706 | p7-298 | GAACCTATCT |
| TY452 | 299 | p5-299 | AGACTTCTGG | 707 | p7-299 | TCGGCGCATA |
| TY384 | 300 | p5-300 | TCCATCTGTA | 708 | p7-300 | GTCACTACTG |
| TY068 | 301 | p5-301 | GAACGGTTAT | 709 | p7-301 | CGGTGTGCAT |
| TY550 | 302 | p5-302 | TGTCAAGTTC | 710 | p7-302 | AGACAATTGG |
| TY065 | 303 | p5-303 | CCGTCAAGGA | 711 | p7-303 | TGACTGCGAA |
| TY139 | 304 | p5-304 | TGGAGGTCCT | 712 | p7-304 | TCAAGCCACC |
| TY363 | 305 | p5-305 | TTCCAAGCAA | 713 | p7-305 | TAATCAGCTG |
| TY374 | 306 | p5-306 | CAGAGGTATT | 714 | p7-306 | GCAGATTGCA |
| TY443 | 307 | p5-307 | AGATCTAGCA | 715 | p7-307 | ATGGAACTGT |
| TY406 | 308 | p5-308 | AAGGTACTGT | 716 | p7-308 | CGTACGACGA |
| TY096 | 309 | p5-309 | GCACTTGGTC | 717 | p7-309 | ATTATCGAGG |
| TY019 | 310 | p5-310 | CGTATCCTGG | 718 | p7-310 | TAATTCCTGC |
| TY030 | 311 | p5-311 | GTAGAATCAC | 719 | p7-311 | AGCCAACGAC |
| TY402 | 312 | p5-312 | CCTAGGAACT | 720 | p7-312 | CTAGGCGGTT |
| TY527 | 313 | p5-313 | TTACAGCGTT | 721 | p7-313 | TCCAGACGAG |
| TY364 | 314 | p5-314 | CGCTGTAAGC | 722 | p7-314 | CGTCTCAACC |
| TY219 | 315 | p5-315 | GAGGCCATAT | 723 | p7-315 | GTATGTACGT |
| TY478 | 316 | p5-316 | ATTGGAACCG | 724 | p7-316 | AACACGCTAA |
| TY163 | 317 | p5-317 | GCTATAGCTA | 725 | p7-317 | GGCGATGAAG |
| TY212 | 318 | p5-318 | AGAGGTTCGT | 726 | p7-318 | CGGATATGTA |
| TY512 | 319 | p5-319 | AACCTCTTCA | 727 | p7-319 | AAGGTTATGC |
| TY208 | 320 | p5-320 | CCATAAGAGT | 728 | p7-320 | ACATCGGTGC |
| TY165 | 321 | p5-321 | AGGTGTTGAT | 729 | p7-321 | TTCAGACCGT |
| TY408 | 322 | p5-322 | TCCGAGGATC | 730 | p7-322 | ACAGATCTCC |
| TY189 | 323 | p5-323 | GAAGCACTAT | 731 | p7-323 | TAACTCTGAG |
| TY537 | 324 | p5-324 | ATTGCAGCCA | 732 | p7-324 | GGTACGAATT |
| TY497 | 325 | p5-325 | CGGATTAAGA | 733 | p7-325 | ATGAGCTAGA |
| TY310 | 326 | p5-326 | CTACTACAGA | 734 | p7-326 | CGTTAAGCAT |
| TY413 | 327 | p5-327 | GGTCACATGG | 735 | p7-327 | TTGTGTTCCT |
| TY080 | 328 | p5-328 | GCGATCTCAC | 736 | p7-328 | ACTGTGAGCG |
| TY526 | 329 | p5-329 | GTCATGTCGT | 737 | p7-329 | ATATGTGTGG |
| TY524 | 330 | p5-330 | TGGCTTATCC | 738 | p7-330 | GACATGTCAT |
| TY046 | 331 | p5-331 | TATTGAGCGT | 739 | p7-331 | TAGCCACATA |
| TY039 | 332 | p5-332 | ATCGCCAGAA | 740 | p7-332 | CGAGGATCAC |
| TY216 | 333 | p5-333 | CCTCAACATT | 741 | p7-333 | ACGCAGTTAG |
| TY190 | 334 | p5-334 | TAAGTCAGTG | 742 | p7-334 | TCACGGAGCT |
| TY237 | 335 | p5-335 | CGCTAGGTAT | 743 | p7-335 | CTTCACAACG |
| TY484 | 336 | p5-336 | CCATGCCTCA | 744 | p7-336 | CGTTATGAGT |
| TY238 | 337 | p5-337 | CAGGAATTGA | 745 | p7-337 | AGAACAGCGT |
| TY070 | 338 | p5-338 | TCAATGCAAC | 746 | p7-338 | TCCTGCAAGG |
| TY532 | 339 | p5-339 | AGACCTATCT | 747 | p7-339 | CTTCAGTCAA |
| TY234 | 340 | p5-340 | TATTCCGATC | 748 | p7-340 | AATAAGCTCC |
| TY543 | 341 | p5-341 | GTCCGTAAGA | 749 | p7-341 | GAAGGCGGAA |
| TY232 | 342 | p5-342 | TCTTGTCCAA | 750 | p7-342 | ACGATTCGAA |
| TY235 | 343 | p5-343 | GTAACGAGCT | 751 | p7-343 | TCCTGCTCTT |
| TY203 | 344 | p5-344 | TGGTGCTTGG | 752 | p7-344 | CTCGAACACG |
| TY347 | 345 | p5-345 | TGCCACAATT | 753 | p7-345 | ACTGTTACAC |
| TY275 | 346 | p5-346 | GCTTCTATGA | 754 | p7-346 | TGACACCACA |
| TY492 | 347 | p5-347 | CAATTGTTCC | 755 | p7-347 | GGTAAGGTCG |
| TY380 | 348 | p5-348 | GTTGCCTAGA | 756 | p7-348 | CTGTCGAGGT |
| TY054 | 349 | p5-349 | TATCAAGCGG | 757 | p7-349 | AAGAGATAGC |
| TY249 | 350 | p5-350 | ATTAGTCGTC | 758 | p7-350 | CCATTACCAA |
| TY485 | 351 | p5-351 | GGTCTAACAT | 759 | p7-351 | CACGGACTTC |
| TY479 | 352 | p5-352 | TGGCAGTAAT | 760 | p7-352 | GTACATTACG |
| TY141 | 353 | p5-353 | CTCTAGTGAT | 761 | p7-353 | TAGGAGACAA |
| TY042 | 354 | p5-354 | TAGCTTGACC | 762 | p7-354 | AGATCTATCG |
| TY181 | 355 | p5-355 | GGAGCAACTG | 763 | p7-355 | TTACTGTGCG |
| TY334 | 356 | p5-356 | TATCACCTCA | 764 | p7-356 | CCGAATCCTC |
| TY233 | 357 | p5-357 | ACGGAACAGG | 765 | p7-357 | GCTGGATTAA |
| TY499 | 358 | p5-358 | GCTCGAGGTA | 766 | p7-358 | ACCGTCTCGT |
| TY292 | 359 | p5-359 | CTGATTAGCC | 767 | p7-359 | AGAGCGGAGA |
| TY006 | 360 | p5-360 | CACTGCGCAA | 768 | p7-360 | GAACACGGAG |
| TY365 | 361 | p5-361 | GGCATCTATT | 769 | p7-361 | AGTCTTGTGA |
| TY441 | 362 | p5-362 | CTATGAGGAA | 770 | p7-362 | TTAAGGCGAG |
| TY494 | 363 | p5-363 | ATGTCTACCA | 771 | p7-363 | TCGTCAATTC |
| TY036 | 364 | p5-364 | TGTTAGGTGA | 772 | p7-364 | CACCACTTGT |
| TY367 | 365 | p5-365 | CCTGATCGCA | 773 | p7-365 | TTCGCAGACT |
| TY273 | 366 | p5-366 | AACAAGAACG | 774 | p7-366 | GAGACTCCGT |
| TY108 | 367 | p5-367 | TACCGAACAC | 775 | p7-367 | TGCGGAGAAC |
| TY362 | 368 | p5-368 | CATTGCTTGC | 776 | p7-368 | ACATATCGCG |
| TY261 | 369 | p5-369 | CGATTGAGTT | 777 | p7-369 | ATCAGGACAG |
| TY371 | 370 | p5-370 | GAGCATGCAA | 778 | p7-370 | GAATTGCGTT |
| TY490 | 371 | p5-371 | TCAGGTTAGC | 779 | p7-371 | TGTGGAGCCT |
| TY521 | 372 | p5-372 | GGTTCAATCA | 780 | p7-372 | AGGAACAAGA |
| TY316 | 373 | p5-373 | AGCAAGCTGC | 781 | p7-373 | CCAAGCTTCT |
| TY236 | 374 | p5-374 | GAACGCTGTC | 782 | p7-374 | TGGCATTGGC |
| TY351 | 375 | p5-375 | ATTCGTCATG | 783 | p7-375 | ACAACGCGGT |
| TY395 | 376 | p5-376 | CAGATCCTAA | 784 | p7-376 | GAGCTACCAC |
| TY003 | 377 | p5-377 | TGCATCCTGA | 785 | p7-377 | TTGTAACCAG |
| TY110 | 378 | p5-378 | GAGGAGAATT | 786 | p7-378 | GCACTATTCT |
| TY376 | 379 | p5-379 | TCTCGATGAA | 787 | p7-379 | ATGGCCAACA |
| TY289 | 380 | p5-380 | AGCGCATAAC | 788 | p7-380 | CATACTACTC |
| TY348 | 381 | p5-381 | CCAATTACCA | 789 | p7-381 | AGCCTGTCCA |
| TY314 | 382 | p5-382 | AGTTCCGGAA | 790 | p7-382 | TCATGTCGGT |
| TY535 | 383 | p5-383 | TTAAGGAAGC | 791 | p7-383 | ACAGGTGGAG |
| TY229 | 384 | p5-384 | CGCAATGTGG | 792 | p7-384 | CAACTCAACT |
| TY217 | 385 | p5-385 | TAAGAAGGCC | 793 | p7-385 | ACTTAGTAGC |
| TY204 | 386 | p5-386 | ATGTGTTCGT | 794 | p7-386 | AGGATATCCA |
| TY458 | 387 | p5-387 | GTTCACCACT | 795 | p7-387 | GCATCAAGAT |
| TY473 | 388 | p5-388 | CCGACGATGA | 796 | p7-388 | AGGAATGTTC |
| TY062 | 389 | p5-389 | AGAATAGAGG | 797 | p7-389 | TTCTACTAGC |
| TY123 | 390 | p5-390 | CTCATTGTCA | 798 | p7-390 | CAGAGGCTAT |
| TY088 | 391 | p5-391 | GCATTCGCTA | 799 | p7-391 | ATTGTGAAGG |
| TY487 | 392 | p5-392 | TGTGAGCTAT | 800 | p7-392 | GTCCTTAACA |
| TY012 | 393 | p5-393 | GAACATAGGT | 801 | p7-393 | AGGCGAGCTT |
| TY034 | 394 | p5-394 | TGGTTGGATA | 802 | p7-394 | TGCTCTCGAT |
| TY483 | 395 | p5-395 | AACACCTGGT | 803 | p7-395 | GAATGGTTCA |
| TY317 | 396 | p5-396 | TATCGATTCG | 804 | p7-396 | ATCGAGAATC |
| TY414 | 397 | p5-397 | TCATTACAGC | 805 | p7-397 | CCAACATTGA |
| TY209 | 398 | p5-398 | TTAGAGCTCA | 806 | p7-398 | ATTCTCCAGT |
| TY315 | 399 | p5-399 | TTGGTGACAA | 807 | p7-399 | GGCATTATCA |
| TY187 | 400 | p5-400 | CTGTGATATC | 808 | p7-400 | ATCGTACATG |
| TY460 | 401 | p5-401 | CAAGTGGTCT | 809 | p7-401 | TGTCTACGGC |
| TY095 | 402 | p5-402 | ATGACTAGGA | 810 | p7-402 | AGAACCAATG |
| TY023 | 403 | p5-403 | TACTGTCGTA | 811 | p7-403 | GTCGACGACA |
| TY143 | 404 | p5-404 | ACACACAAGG | 812 | p7-404 | CTTGGTATGT |
| TY516 | 405 | p5-405 | TGCCTAGCGT | 813 | p7-405 | GACTTATCCT |
| TY058 | 406 | p5-406 | GCTATCCTCT | 814 | p7-406 | GCGTGAATCA |
| TY044 | 407 | p5-407 | TGCTAGTTGT | 815 | p7-407 | CTCCTGTTAT |
| TY138 | 408 | p5-408 | GTACACGGAC | 816 | p7-408 | ATATTAGCGC |

2. The method according to claim 1, wherein performing library construction on the target sample utilizing the primer with 5′ phosphorylation modification, obtaining the linear amplification library with 5′ phosphorylation modification comprises:
performing adapter ligation on a fragment obtained from the target sample utilizing truncated adapters shown in SEQ ID NO: 817 and SEQ ID NO: 818, obtaining a fragment with adapters; and
amplifying the fragment with the adapters utilizing the P5 truncated amplification primer and the P7 truncated amplification primer, obtaining the linear amplification library with 5′ phosphorylation modification, wherein
a sequence of SEQ ID NO: 817 is
5′-ACACTCTTTCCCTACACGACGCTCTTCCGATC*T-3′, and * represents thio modification;
and
a sequence of SEQ ID NO: 818 is
5′-GATCGGAAGAGCACACGTCTGAACTCCAGTCAC-3′, and a 5′ end is modified through
phosphorylation.

3. The method according to claim 1, wherein performing library construction on the target sample utilizing the adapter with 5′ phosphorylation modification, and obtaining the linear amplification library with 5′ phosphorylation modification comprises:
performing adapter ligation on a fragment obtained from the target sample utilizing the P5 full-length adapter and the P7 full-length adapter, obtaining a fragment with adapters; and
amplifying the fragment with the adapters utilizing library amplification primers shown in SEQ ID NO: 822 and SEQ ID NO: 825, obtaining the linear amplification library with 5′ phosphorylation modification, wherein
a sequence of SEQ ID NO: 822 is
5′-AATGATACGGCGACCACCGAGAT-3′, and a 5′ end is modified through phosphorylation;
and
a sequence of SEQ ID NO: 825 is
5′-CAAGCAGAAGACGGCATACGA-3′.

4. The method according to claim 1, wherein before circularization, the method further comprises a step of performing targeted capture on the linear amplification library.

5. The method according to claim 4, wherein a captured library after targeted capture is amplified with targeted library amplification primers, and obtaining a linear amplified captured library; then circularizing the linear amplified captured library, and obtaining the circularized library configured for sequencing on a platform utilizing DNA nanoball generation and combinatorial probe anchor polymerization.

6. The method according to claim 5, wherein the targeted library amplification primers have nucleotide sequences shown in SEQ ID NO: 822 and SEQ ID NO: 825, wherein
a sequence of SEQ ID NO: 822 is
5′-AATGATACGGCGACCACCGAGAT-3′, and a 5′ end is modified through phosphorylation;
and
a sequence of SEQ ID NO: 825 is
5′-CAAGCAGAAGACGGCATACGA-3′

7. A kit for constructing a DNA library, comprising any one of the following combinations:
1) Combination 1: a P5 truncated amplification primer in the method for constructing a DNA library according to claim 1 and a P7 truncated amplification primer in the method for constructing a DNA library according to claim 1; and
2) Combination 2: a P5 full-length adapter in the method for constructing a DNA library according to claim 1 and a P7 full-length adapter in the method for constructing a DNA library according to claim 1, wherein
the kit for constructing a DNA library comprises 408 P5-end index sequences and 408 corresponding fixedly-matched P7-end index sequences, the P5-end index sequences are shown in Table 1 in the method for constructing a DNA library according to claim 1, and the P7-end index sequences are shown in Table 1 in the method for constructing a DNA library according to claim 1;
the P5-end index sequence is used in conjunction with a group of 8-base balanced index sequences, and the P7-end index sequence is used according to the fixedly-matched P7-end index sequences corresponding to the P5-end index sequences.

8. The kit according to claim 7, further comprising library amplification primers shown in SEQ ID NO: 822 and SEQ ID NO: 825, and/or truncated adapters shown in SEQ ID NO: 817 and SEQ ID NO: 818.

9. An adapter element compatible with dual-sequencing platforms, wherein the adapter element is selected from any one of the following combinations:
1) Combination 1: a P5 truncated amplification primer in the method for constructing a DNA library according to claim 1 and a P7 truncated amplification primer in the method for constructing a DNA library according to claim 1; and
2) Combination 2: a P5 full-length adapter in the method for constructing a DNA library according to claim 1 and a P7 full-length adapter in the method for constructing a DNA library according to claim 1, wherein
the sequences, which are 1 bp from upstream and downstream of the index sequence comprising the P5-end index sequence or the P7-end index sequence, contain at least three edit distances;
the adapter element is an amplification primer composition or an adapter composition;
the amplification primer composition comprises a combination of the P5 truncated amplification primers and/or the P7 truncated amplification primers; the P5 truncated amplification primers and the P7 truncated amplification primers are each independently of a group or a plurality of groups; each group of the P5 truncated amplification primers comprises P5-end index sequences selected from any one of loading combinations in Table 1 in the method for constructing a DNA library according to claim 1; each group of the P7 truncated amplification primers comprises fixedly-matched P7-end index sequences corresponding to the P5-end index sequences selected from Table 1 in the method for constructing a DNA library according to claim 1; the respective number of bases A, T, C, and G in the loading combination is ≥12.5%;
the adapter composition comprises a plurality of groups of the P5 full-length adapters and/or a plurality of groups of the P7 full-length adapters; the P5 full-length adapters and the P7 full-length adapters are each independently of a group or a plurality of groups; each group of the P5 full-length adapters comprises P5-end index sequences selected from any one of loading combinations in Table 1 in the method for constructing a DNA library according to claim 1; each group of the P7 full-length adapters comprises fixedly-matched P7-end index sequences corresponding to the P5-end index sequences selected from Table 1 in the method for constructing a DNA library according to claim 1; the respective number of bases A, T, C, and G in the loading combination is ≥12.5%; and 8 indexes constitute a group of the loading combinations.


SEQUENCE LISTING (825 sequences)

SEQ ID NO: 1 - DNA, 10 bp, synthetic construct
  source 1..10: mol_type = other DNA
gacctcggtt

SEQ ID NO: 2 - DNA, 10 bp, synthetic construct
  source 1..10: mol_type = other DNA
cggaagctga

SEQ ID NO: 3 - DNA, 10 bp, synthetic construct
  source 1..10: mol_type = other DNA
acgcgttcaa

SEQ ID NO: 4 - DNA, 10 bp, synthetic construct
  source 1..10: mol_type = other DNA
gttgcaagtc

SEQ ID NO: 5 - DNA, 10 bp, synthetic construct
  source 1..10: mol_type = other DNA
ttctggatcg

SEQ ID NO: 6 - DNA, 10 bp, synthetic construct
  source 1..10: mol_type = other DNA
gcagaacatt

SEQ ID NO: 7 - DNA, 10 bp, synthetic construct
  source 1..10: mol_type = other DNA
aattcctacc

SEQ ID NO: 8 - DNA, 10 bp, synthetic construct
  source 1..10: mol_type = other DNA
tgaagaccaa

SEQ ID NO: 9 - DNA, 10 bp, synthetic construct
  source 1..10: mol_type = other DNA
cttctgagta

SEQ ID NO: 10 - DNA, 10 bp, synthetic construct
  source 1..10: mol_type = other DNA
aagtgccagg

SEQ ID NO: 11 - DNA, 10 bp, synthetic construct
  source 1..10: mol_type = other DNA
gcaagtatac

SEQ ID NO: 12 - DNA, 10 bp, synthetic construct
  source 1..10: mol_type = other DNA
tggcaatggc

SEQ ID NO: 13 - DNA, 10 bp, synthetic construct
  source 1..10: mol_type = other DNA
ttcgttacct

SEQ ID NO: 14 - DNA, 10 bp, synthetic construct
  source 1..10: mol_type = other DNA
cttgcggtaa

SEQ ID NO: 15 - DNA, 10 bp, synthetic construct
  source 1..10: mol_type = other DNA
ggaacacatt

SEQ ID NO: 16 - DNA, 10 bp, synthetic construct
  source 1..10: mol_type = other DNA
acatagtcct

SEQ ID NO: 17 - DNA, 10 bp, synthetic construct
  source 1..10: mol_type = other DNA
gagatcttgt

SEQ ID NO: 18 - DNA, 10 bp, synthetic construct
  source 1..10: mol_type = other DNA
cgtgctaacc

SEQ ID NO: 19 - DNA, 10 bp, synthetic construct
  source 1..10: mol_type = other DNA
ataaggcctc

SEQ ID NO: 20 - DNA, 10 bp, synthetic construct
  source 1..10: mol_type = other DNA
tcgtacggaa

SEQ ID NO: 21 - DNA, 10 bp, synthetic construct
  source 1..10: mol_type = other DNA
attccaccga

SEQ ID NO: 22 - DNA, 10 bp, synthetic construct
  source 1..10: mol_type = other DNA
tacgtactct

SEQ ID NO: 23 - DNA, 10 bp, synthetic construct
  source 1..10: mol_type = other DNA
ggccttagag

SEQ ID NO: 24 - DNA, 10 bp, synthetic construct
  source 1..10: mol_type = other DNA
ccaagtgaga

SEQ ID NO: 25 - DNA, 10 bp, synthetic construct
  source 1..10: mol_type = other DNA
ggttgtaact

SEQ ID NO: 26 - DNA, 10 bp, synthetic construct
  source 1..10: mol_type = other DNA
ctcctcgcaa

SEQ ID NO: 27 - DNA, 10 bp, synthetic construct
  source 1..10: mol_type = other DNA
tatgactgtg

SEQ ID NO: 28 - DNA, 10 bp, synthetic construct
  source 1..10: mol_type = other DNA
agaacagtcc

SEQ ID NO: 29 - DNA, 10 bp, synthetic construct
  source 1..10: mol_type = other DNA
atgctgcgga

SEQ ID NO: 30 - DNA, 10 bp, synthetic construct
  source 1..10: mol_type = other DNA
gactcacagc

SEQ ID NO: 31 - DNA, 10 bp, synthetic construct
  source 1..10: mol_type = other DNA
tctcggttat

SEQ ID NO: 32 - DNA, 10 bp, synthetic construct
  source 1..10: mol_type = other DNA
ggatatacca

SEQ ID NO: 33 - DNA, 10 bp, synthetic construct
  source 1..10: mol_type = other DNA
cgcagatcca

SEQ ID NO: 34 - DNA, 10 bp, synthetic construct
  source 1..10: mol_type = other DNA
tagcctatgg

SEQ ID NO: 35 - DNA, 10 bp, synthetic construct
  source 1..10: mol_type = other DNA
actgaccata

SEQ ID NO: 36 - DNA, 10 bp, synthetic construct
  source 1..10: mol_type = other DNA
ggctactgct

SEQ ID NO: 37 - DNA, 10 bp, synthetic construct
  source 1..10: mol_type = other DNA
ctagtgtacc

SEQ ID NO: 38 - DNA, 10 bp, synthetic construct
  source 1..10: mol_type = other DNA
tcttcggagg

SEQ ID NO: 39 - DNA, 10 bp, synthetic construct
  source 1..10: mol_type = other DNA
ttgaccagac

SEQ ID NO: 40 - DNA, 10 bp, synthetic construct
  source 1..10: mol_type = other DNA
caacttgcaa

SEQ ID NO: 41 - DNA, 10 bp, synthetic construct
  source 1..10: mol_type = other DNA
ggttgatcga

SEQ ID NO: 42 - DNA, 10 bp, synthetic construct
  source 1..10: mol_type = other DNA
ataatggtcg

SEQ ID NO: 43 - DNA, 10 bp, synthetic construct
  source 1..10: mol_type = other DNA
gacaaccatt

SEQ ID NO: 44 - DNA, 10 bp, synthetic construct
  source 1..10: mol_type = other DNA
acggtcacac

SEQ ID NO: 45 - DNA, 10 bp, synthetic construct
  source 1..10: mol_type = other DNA
cggccaatgt

SEQ ID NO: 46 - DNA, 10 bp, synthetic construct
  source 1..10: mol_type = other DNA
tggaattgtc

SEQ ID NO: 47 - DNA, 10 bp, synthetic construct
  source 1..10: mol_type = other DNA
caacgagtcg

SEQ ID NO: 48 - DNA, 10 bp, synthetic construct
  source 1..10: mol_type = other DNA
acctccacaa

SEQ ID NO: 49 - DNA, 10 bp, synthetic construct
  source 1..10: mol_type = other DNA
ggaatctcct

SEQ ID NO: 50 - DNA, 10 bp, synthetic construct
  source 1..10: mol_type = other DNA
aagcgaggaa

SEQ ID NO: 51 - DNA, 10 bp, synthetic construct
  source 1..10: mol_type = other DNA
cattagatcg

SEQ ID NO: 52 - DNA, 10 bp, synthetic construct
  source 1..10: mol_type = other DNA
ttcttctagc

SEQ ID NO: 53 - DNA, 10 bp, synthetic construct
  source 1..10: mol_type = other DNA
attgctccta

SEQ ID NO: 54 - DNA, 10 bp, synthetic construct
  source 1..10: mol_type = other DNA
gcgataatct

SEQ ID NO: 55 - DNA, 10 bp, synthetic construct
  source 1..10: mol_type = other DNA
tgtcgtagtg

SEQ ID NO: 56 - DNA, 10 bp, synthetic construct
  source 1..10: mol_type = other DNA
aagtccgaac

SEQ ID NO: 57 - DNA, 10 bp, synthetic construct
  source 1..10: mol_type = other DNA
caagtcacaa

SEQ ID NO: 58 - DNA, 10 bp, synthetic construct
  source 1..10: mol_type = other DNA
gcctcatggt

SEQ ID NO: 59 - DNA, 10 bp, synthetic construct
  source 1..10: mol_type = other DNA
tggcgtctgt

SEQ ID NO: 60 - DNA, 10 bp, synthetic construct
  source 1..10: mol_type = other DNA
actcaggacg

SEQ ID NO: 61 - DNA, 10 bp, synthetic construct
  source 1..10: mol_type = other DNA
tagagaggtc

SEQ ID NO: 62 - DNA, 10 bp, synthetic construct
  source 1..10: mol_type = other DNA
gtctcgcttc

SEQ ID NO: 63 - DNA, 10 bp, synthetic construct
  source 1..10: mol_type = other DNA
cttagctcaa

SEQ ID NO: 64 - DNA, 10 bp, synthetic construct
  source 1..10: mol_type = other DNA
gcacagaact

SEQ ID NO: 65 - DNA, 10 bp, synthetic construct
  source 1..10: mol_type = other DNA
cgttgcatct

SEQ ID NO: 66 - DNA, 10 bp, synthetic construct
  source 1..10: mol_type = other DNA
atgaatgcga

SEQ ID NO: 67 - DNA, 10 bp, synthetic construct
  source 1..10: mol_type = other DNA
acactatgtg

SEQ ID NO: 68 - DNA, 10 bp, synthetic construct
  source 1..10: mol_type = other DNA
tgctagagcc

SEQ ID NO: 69 - DNA, 10 bp, synthetic construct
  source 1..10: mol_type = other DNA
cagctaccac

SEQ ID NO: 70 - DNA, 10 bp, synthetic construct
  source 1..10: mol_type = other DNA
gtagccaata

SEQ ID NO: 71 - DNA, 10 bp, synthetic construct
  source 1..10: mol_type = other DNA
gatggattgt

SEQ ID NO: 72 - DNA, 10 bp, synthetic construct
  source 1..10: mol_type = other DNA
tccatggagc

SEQ ID NO: 73 - DNA, 10 bp, synthetic construct
  source 1..10: mol_type = other DNA
gaataagcca

SEQ ID NO: 74 - DNA, 10 bp, synthetic construct
  source 1..10: mol_type = other DNA
agtatgctga

SEQ ID NO: 75 - DNA, 10 bp, synthetic construct
  source 1..10: mol_type = other DNA
ttctactgtc

SEQ ID NO: 76 - DNA, 10 bp, synthetic construct
  source 1..10: mol_type = other DNA
ccggaatatt

SEQ ID NO: 77 - DNA, 10 bp, synthetic construct
  source 1..10: mol_type = other DNA
gagcggtaag

SEQ ID NO: 78 - DNA, 10 bp, synthetic construct
  source 1..10: mol_type = other DNA
gtagctaggc

SEQ ID NO: 79 - DNA, 10 bp, synthetic construct
  source 1..10: mol_type = other DNA
cacattacgt

SEQ ID NO: 80 - DNA, 10 bp, synthetic construct
  source 1..10: mol_type = other DNA
tccttcgtta

SEQ ID NO: 81 - DNA, 10 bp, synthetic construct
  source 1..10: mol_type = other DNA
cgtggtgcta

SEQ ID NO: 82 - DNA, 10 bp, synthetic construct
  source 1..10: mol_type = other DNA
acgacaagac

SEQ ID NO: 83 - DNA, 10 bp, synthetic construct
  source 1..10: mol_type = other DNA
gaatacctcg

SEQ ID NO: 84 - DNA, 10 bp, synthetic construct
  source 1..10: mol_type = other DNA
tggctggtgt

SEQ ID NO: 85 - DNA, 10 bp, synthetic construct
  source 1..10: mol_type = other DNA
cttcaacgac

SEQ ID NO: 86 - DNA, 10 bp, synthetic construct
  source 1..10: mol_type = other DNA
tccacgtagg

SEQ ID NO: 87 - DNA, 10 bp, synthetic construct
  source 1..10: mol_type = other DNA
gtcaggcaca

SEQ ID NO: 88 - DNA, 10 bp, synthetic construct
  source 1..10: mol_type = other DNA
ggattcactt

SEQ ID NO: 89 - DNA, 10 bp, synthetic construct
  source 1..10: mol_type = other DNA
aagctccata

SEQ ID NO: 90 - DNA, 10 bp, synthetic construct
  source 1..10: mol_type = other DNA
gtcaatatcc

SEQ ID NO: 91 - DNA, 10 bp, synthetic construct
  source 1..10: mol_type = other DNA
ctatcggcct

SEQ ID NO: 92 - DNA, 10 bp, synthetic construct
  source 1..10: mol_type = other DNA
ggtcaaggtg

SEQ ID NO: 93 - DNA, 10 bp, synthetic construct
  source 1..10: mol_type = other DNA
caggtgcagt

SEQ ID NO: 94 - DNA, 10 bp, synthetic construct
  source 1..10: mol_type = other DNA
tcacgactaa

SEQ ID NO: 95 - DNA, 10 bp, synthetic construct
  source 1..10: mol_type = other DNA
atcggttgaa

SEQ ID NO: 96 - DNA, 10 bp, synthetic construct
  source 1..10: mol_type = other DNA
acttcattgc

SEQ ID NO: 97 - DNA, 10 bp, synthetic construct
  source 1..10: mol_type = other DNA
ggcgagacaa

SEQ ID NO: 98 - DNA, 10 bp, synthetic construct
  source 1..10: mol_type = other DNA
ctgccttgtg

SEQ ID NO: 99 - DNA, 10 bp, synthetic construct
  source 1..10: mol_type = other DNA
accttcaagt

SEQ ID NO: 100 - DNA, 10 bp, synthetic construct
  source 1..10: mol_type = other DNA
ataacgcgca

SEQ ID NO: 101 - DNA, 10 bp, synthetic construct
  source 1..10: mol_type = other DNA
tgtggagtcc

SEQ ID NO: 102 - DNA, 10 bp, synthetic construct
  source 1..10: mol_type = other DNA
gaacttcgga

SEQ ID NO: 103 - DNA, 10 bp, synthetic construct
  source 1..10: mol_type = other DNA
cataacttcg

SEQ ID NO: 104 - DNA, 10 bp, synthetic construct
  source 1..10: mol_type = other DNA
tcctggcatt

SEQ ID NO: 105 - DNA, 10 bp, synthetic construct
  source 1..10: mol_type = other DNA
cgatgcacga

SEQ ID NO: 106 - DNA, 10 bp, synthetic construct
  source 1..10: mol_type = other DNA
actatacacc

SEQ ID NO: 107 - DNA, 10 bp, synthetic construct
  source 1..10: mol_type = other DNA
cgacctgtgt

SEQ ID NO: 108 - DNA, 10 bp, synthetic construct
  source 1..10: mol_type = other DNA
gacagttgta

SEQ ID NO: 109 - DNA, 10 bp, synthetic construct
  source 1..10: mol_type = other DNA
ttggctgcct

SEQ ID NO: 110 - DNA, 10 bp, synthetic construct
  source 1..10: mol_type = other DNA
cacaaccgag

SEQ ID NO: 111 - DNA, 10 bp, synthetic construct
  source 1..10: mol_type = other DNA
actgagtaac

SEQ ID NO: 112 - DNA, 10 bp, synthetic construct
  source 1..10: mol_type = other DNA
gtcctagtaa

SEQ ID NO: 113 - DNA, 10 bp, synthetic construct
  source 1..10: mol_type = other DNA
gatctcgata

SEQ ID NO: 114 - DNA, 10 bp, synthetic construct
  source 1..10: mol_type = other DNA
tgatcttcgc

SEQ ID NO: 115 - DNA, 10 bp, synthetic construct
  source 1..10: mol_type = other DNA
tctcagcgct

SEQ ID NO: 116 - DNA, 10 bp, synthetic construct
  source 1..10: mol_type = other DNA
acgagagtgg

SEQ ID NO: 117 - DNA, 10 bp, synthetic construct
  source 1..10: mol_type = other DNA
cgcgtcagaa

SEQ ID NO: 118 - DNA, 10 bp, synthetic construct
  source 1..10: mol_type = other DNA
gtctctggat

SEQ ID NO: 119 - DNA, 10 bp, synthetic construct
  source 1..10: mol_type = other DNA
cagtggaatc

SEQ ID NO: 120 - DNA, 10 bp, synthetic construct
  source 1..10: mol_type = other DNA
acttgtctgt

SEQ ID NO: 121 - DNA, 10 bp, synthetic construct
  source 1..10: mol_type = other DNA
agctgtacaa

SEQ ID NO: 122 - DNA, 10 bp, synthetic construct
  source 1..10: mol_type = other DNA
ctagcatgat

SEQ ID NO: 123 - DNA, 10 bp, synthetic construct
  source 1..10: mol_type = other DNA
tagattctcc

SEQ ID NO: 124 - DNA, 10 bp, synthetic construct
  source 1..10: mol_type = other DNA
acatacgatg

SEQ ID NO: 125 - DNA, 10 bp, synthetic construct
  source 1..10: mol_type = other DNA
tgtcggtgga

SEQ ID NO: 126 - DNA, 10 bp, synthetic construct
  source 1..10: mol_type = other DNA
ctggatgcca

SEQ ID NO: 127 - DNA, 10 bp, synthetic construct
  source 1..10: mol_type = other DNA
gctagacagg

SEQ ID NO: 128 - DNA, 10 bp, synthetic construct
  source 1..10: mol_type = other DNA
agcatgttcc

SEQ ID NO: 129 - DNA, 10 bp, synthetic construct
  source 1..10: mol_type = other DNA
tacggaagaa

SEQ ID NO: 130 - DNA, 10 bp, synthetic construct
  source 1..10: mol_type = other DNA
cgactgactc

SEQ ID NO: 131 - DNA, 10 bp, synthetic construct
  source 1..10: mol_type = other DNA
gcttgcttat

SEQ ID NO: 132 - DNA, 10 bp, synthetic construct
  source 1..10: mol_type = other DNA
aggccacaga

SEQ ID NO: 133 - DNA, 10 bp, synthetic construct
  source 1..10: mol_type = other DNA
gacaatgagt

SEQ ID NO: 134 - DNA, 10 bp, synthetic construct
  source 1..10: mol_type = other DNA
atctccggtg

SEQ ID NO: 135 - DNA, 10 bp, synthetic construct
  source 1..10: mol_type = other DNA
tctactcgcg

SEQ ID NO: 136 - DNA, 10 bp, synthetic construct
  source 1..10: mol_type = other DNA
agagtctctt

SEQ ID NO: 137 - DNA, 10 bp, synthetic construct
  source 1..10: mol_type = other DNA
cactgcaatt

SEQ ID NO: 138 - DNA, 10 bp, synthetic construct
  source 1..10: mol_type = other DNA
actgacttga

SEQ ID NO: 139 - DNA, 10 bp, synthetic construct
  source 1..10: mol_type = other DNA
taaccggacc

SEQ ID NO: 140 - DNA, 10 bp, synthetic construct
  source 1..10: mol_type = other DNA
tccattccaa

SEQ ID NO: 141 - DNA, 10 bp, synthetic construct
  source 1..10: mol_type = other DNA
ttgtgaggcg

SEQ ID NO: 142 - DNA, 10 bp, synthetic construct
  source 1..10: mol_type = other DNA
ggagagttat

SEQ ID NO: 143 - DNA, 10 bp, synthetic construct
  source 1..10: mol_type = other DNA
aaggatagcc

SEQ ID NO: 144 - DNA, 10 bp, synthetic construct
  source 1..10: mol_type = other DNA
tgtccggcta

SEQ ID NO: 145 - DNA, 10 bp, synthetic construct
  source 1..10: mol_type = other DNA
ctgctcgtct

SEQ ID NO: 146 - DNA, 10 bp, synthetic construct
  source 1..10: mol_type = other DNA
gctagtcgaa

SEQ ID NO: 147 - DNA, 10 bp, synthetic construct
  source 1..10: mol_type = other DNA
tgagagtcgc

SEQ ID NO: 148 - DNA, 10 bp, synthetic construct
  source 1..10: mol_type = other DNA
agataacctg

SEQ ID NO: 149 - DNA, 10 bp, synthetic construct
  source 1..10: mol_type = other DNA
ttcgcggagt

SEQ ID NO: 150 - DNA, 10 bp, synthetic construct
  source 1..10: mol_type = other DNA
cagtatagga

SEQ ID NO: 151 - DNA, 10 bp, synthetic construct
  source 1..10: mol_type = other DNA
tagagcattc

SEQ ID NO: 152 - DNA, 10 bp, synthetic construct
  source 1..10: mol_type = other DNA
atcctaacct

SEQ ID NO: 153 - DNA, 10 bp, synthetic construct
  source 1..10: mol_type = other DNA
gttaagtggt

SEQ ID NO: 154 - DNA, 10 bp, synthetic construct
  source 1..10: mol_type = other DNA
cggctcctta

SEQ ID NO: 155 - DNA, 10 bp, synthetic construct
  source 1..10: mol_type = other DNA
tactgaatcc

SEQ ID NO: 156 - DNA, 10 bp, synthetic construct
  source 1..10: mol_type = other DNA
acagcaacaa

SEQ ID NO: 157 - DNA, 10 bp, synthetic construct
  source 1..10: mol_type = other DNA
aggagttaac

SEQ ID NO: 158 - DNA, 10 bp, synthetic construct
  source 1..10: mol_type = other DNA
taacaggcta

SEQ ID NO: 159 - DNA, 10 bp, synthetic construct
  source 1..10: mol_type = other DNA
ccttgtcagg

SEQ ID NO: 160 - DNA, 10 bp, synthetic construct
  source 1..10: mol_type = other DNA
ttggtatgct

SEQ ID NO: 161 - DNA, 10 bp, synthetic construct
  source 1..10: mol_type = other DNA
aacgtaagca

SEQ ID NO: 162 - DNA, 10 bp, synthetic construct
  source 1..10: mol_type = other DNA
ccacagctgt

SEQ ID NO: 163 - DNA, 10 bp, synthetic construct
  source 1..10: mol_type = other DNA
tatagcgaac

SEQ ID NO: 164 - DNA, 10 bp, synthetic construct
  source 1..10: mol_type = other DNA
ggtccttcac

SEQ ID NO: 165 - DNA, 10 bp, synthetic construct
  source 1..10: mol_type = other DNA
ttgcgagata

SEQ ID NO: 166 - DNA, 10 bp, synthetic construct
  source 1..10: mol_type = other DNA
gaatccgact

SEQ ID NO: 167 - DNA, 10 bp, synthetic construct
  source 1..10: mol_type = other DNA
ctgattctag

SEQ ID NO: 168 - DNA, 10 bp, synthetic construct
  source 1..10: mol_type = other DNA
acggcgattg

SEQ ID NO: 169 - DNA, 10 bp, synthetic construct
  source 1..10: mol_type = other DNA
tgacggagta

SEQ ID NO: 170 - DNA, 10 bp, synthetic construct
  source 1..10: mol_type = other DNA
attgatgagc

SEQ ID NO: 171 - DNA, 10 bp, synthetic construct
  source 1..10: mol_type = other DNA
ggctagccat

SEQ ID NO: 172 - DNA, 10 bp, synthetic construct
  source 1..10: mol_type = other DNA
tatgccttcg

SEQ ID NO: 173 - DNA, 10 bp, synthetic construct
  source 1..10: mol_type = other DNA
ccgatccatt

SEQ ID NO: 174 - DNA, 10 bp, synthetic construct
  source 1..10: mol_type = other DNA
ggtgtataga

SEQ ID NO: 175 - DNA, 10 bp, synthetic construct
  source 1..10: mol_type = other DNA
ctgtctgcac

SEQ ID NO: 176 - DNA, 10 bp, synthetic construct
  source 1..10: mol_type = other DNA
tcaccaatca

SEQ ID NO: 177 - DNA, 10 bp, synthetic construct
  source 1..10: mol_type = other DNA
tgagaacggt

SEQ ID NO: 178 - DNA, 10 bp, synthetic construct
  source 1..10: mol_type = other DNA
gtcctgaaca

SEQ ID NO: 179 - DNA, 10 bp, synthetic construct
  source 1..10: mol_type = other DNA
acgagttgcc

SEQ ID NO: 180 - DNA, 10 bp, synthetic construct
  source 1..10: mol_type = other DNA
cattaagctg

SEQ ID NO: 181 - DNA, 10 bp, synthetic construct
  source 1..10: mol_type = other DNA
ctgcacctac

SEQ ID NO: 182 - DNA, 10 bp, synthetic construct
  source 1..10: mol_type = other DNA
cgctctgatg

SEQ ID NO: 183 - DNA, 10 bp, synthetic construct
  source 1..10: mol_type = other DNA
gctgactatt

SEQ ID NO: 184 - DNA, 10 bp, synthetic construct
  source 1..10: mol_type = other DNA
tacttggcta

SEQ ID NO: 185 - DNA, 10 bp, synthetic construct
  source 1..10: mol_type = other DNA
catcagccaa

SEQ ID NO: 186 - DNA, 10 bp, synthetic construct
  source 1..10: mol_type = other DNA
gccttgtatt

SEQ ID NO: 187 - DNA, 10 bp, synthetic construct
  source 1..10: mol_type = other DNA
ttgactatcg

SEQ ID NO: 188 - DNA, 10 bp, synthetic construct
  source 1..10: mol_type = other DNA
aattgtaggc

SEQ ID NO: 189 - DNA, 10 bp, synthetic construct
  source 1..10: mol_type = other DNA
aggtccgtag

SEQ ID NO: 190 - DNA, 10 bp, synthetic construct
  source 1..10: mol_type = other DNA
caagaacacc

SEQ ID NO: 191 - DNA, 10 bp, synthetic construct
  source 1..10: mol_type = other DNA
cctagatgta

SEQ ID NO: 192 - DNA, 10 bp, synthetic construct
  source 1..10: mol_type = other DNA
tgcttcgaat

SEQ ID NO: 193 - DNA, 10 bp, synthetic construct
  source 1..10: mol_type = other DNA
aagcttggat

SEQ ID NO: 194 - DNA, 10 bp, synthetic construct
  source 1..10: mol_type = other DNA
tcctggttcc

SEQ ID NO: 195 - DNA, 10 bp, synthetic construct
  source 1..10: mol_type = other DNA
gctatcaaga

SEQ ID NO: 196 - DNA, 10 bp, synthetic construct
  source 1..10: mol_type = other DNA
gtcggactgt

SEQ ID NO: 197 - DNA, 10 bp, synthetic construct
  source 1..10: mol_type = other DNA
ccataacctt

SEQ ID NO: 198 - DNA, 10 bp, synthetic construct
  source 1..10: mol_type = other DNA
agtactccgc

SEQ ID NO: 199 - DNA, 10 bp, synthetic construct
  source 1..10: mol_type = other DNA
ttggcggttg

SEQ ID NO: 200 - DNA, 10 bp, synthetic construct
  source 1..10: mol_type = other DNA
agttatagcg

SEQ ID NO: 201 - DNA, 10 bp, synthetic construct
  source 1..10: mol_type = other DNA
tgacgcggat

SEQ ID NO: 202 - DNA, 10 bp, synthetic construct
  source 1..10: mol_type = other DNA
cagaataacc

SEQ ID NO: 203 - DNA, 10 bp, synthetic construct
  source 1..10: mol_type = other DNA
atggcacgga

SEQ ID NO: 204 - DNA, 10 bp, synthetic construct
  source 1..10: mol_type = other DNA
ggcagcttaa

SEQ ID NO: 205 - DNA, 10 bp, synthetic construct
  source 1..10: mol_type = other DNA
cctgttgtgt

SEQ ID NO: 206 - DNA, 10 bp, synthetic construct
  source 1..10: mol_type = other DNA
cactcgacca

SEQ ID NO: 207 - DNA, 10 bp, synthetic construct
  source 1..10: mol_type = other DNA
acacgtcgtg

SEQ ID NO: 208 - DNA, 10 bp, synthetic construct
  source 1..10: mol_type = other DNA
ggtatgcacc

SEQ ID NO: 209 - DNA, 10 bp, synthetic construct
  source 1..10: mol_type = other DNA
ttgaaccgct

SEQ ID NO: 210 - DNA, 10 bp, synthetic construct
  source 1..10: mol_type = other DNA
cgaactaagc

SEQ ID NO: 211 - DNA, 10 bp, synthetic construct
  source 1..10: mol_type = other DNA
ggttaagctt

SEQ ID NO: 212 - DNA, 10 bp, synthetic construct
  source 1..10: mol_type = other DNA
ggcggatgaa

SEQ ID NO: 213 - DNA, 10 bp, synthetic construct
  source 1..10: mol_type = other DNA
ttgctgattg

SEQ ID NO: 214 - DNA, 10 bp, synthetic construct
  source 1..10: mol_type = other DNA
cctcattaga

SEQ ID NO: 215 - DNA, 10 bp, synthetic construct
  source 1..10: mol_type = other DNA
aaggcgacca

SEQ ID NO: 216 - DNA, 10 bp, synthetic construct
  source 1..10: mol_type = other DNA
atctgtccgc

SEQ ID NO: 217 - DNA, 10 bp, synthetic construct
  source 1..10: mol_type = other DNA
cggtaatgat

SEQ ID NO: 218 - DNA, 10 bp, synthetic construct
  source 1..10: mol_type = other DNA
ctcgttgcga

SEQ ID NO: 219 - DNA, 10 bp, synthetic construct
  source 1..10: mol_type = other DNA
gcacacatga

SEQ ID NO: 220 - DNA, 10 bp, synthetic construct
  source 1..10: mol_type = other DNA
tacaatcctc

SEQ ID NO: 221 - DNA, 10 bp, synthetic construct
  source 1..10: mol_type = other DNA
tctccgtacc

SEQ ID NO: 222 - DNA, 10 bp, synthetic construct
  source 1..10: mol_type = other DNA
aacgtctaag

SEQ ID NO: 223 - DNA, 10 bp, synthetic construct
  source 1..10: mol_type = other DNA
agtggcgtct

SEQ ID NO: 224 - DNA, 10 bp, synthetic construct
  source 1..10: mol_type = other DNA
gtcttaggtt

SEQ ID NO: 225 - DNA, 10 bp, synthetic construct
  source 1..10: mol_type = other DNA
aatagctgca

SEQ ID NO: 226 - DNA, 10 bp, synthetic construct
  source 1..10: mol_type = other DNA
gtctcagaag

SEQ ID NO: 227 - DNA, 10 bp, synthetic construct
  source 1..10: mol_type = other DNA
tcagtgctcc

SEQ ID NO: 228 - DNA, 10 bp, synthetic construct
  source 1..10: mol_type = other DNA
cgtcatactt

SEQ ID NO: 229 - DNA, 10 bp, synthetic construct
  source 1..10: mol_type = other DNA
tcggattggc

SEQ ID NO: 230 - DNA, 10 bp, synthetic construct
  source 1..10: mol_type = other DNA
aatggagcaa

SEQ ID NO: 231 - DNA, 10 bp, synthetic construct
  source 1..10: mol_type = other DNA
cggttcatgt

SEQ ID NO: 232 - DNA, 10 bp, synthetic construct
  source 1..10: mol_type = other DNA
gccagttatt

SEQ ID NO: 233 - DNA, 10 bp, synthetic construct
  source 1..10: mol_type = other DNA
ctgcgttaca

SEQ ID NO: 234 - DNA, 10 bp, synthetic construct
  source 1..10: mol_type = other DNA
tgagtagcat

SEQ ID NO: 235 - DNA, 10 bp, synthetic construct
  source 1..10: mol_type = other DNA
gactcgttga

SEQ ID NO: 236 - DNA, 10 bp, synthetic construct
  source 1..10: mol_type = other DNA
ctacatccgg

SEQ ID NO: 237 - DNA, 10 bp, synthetic construct
  source 1..10: mol_type = other DNA
gataccggtc

SEQ ID NO: 238 - DNA, 10 bp, synthetic construct
  source 1..10: mol_type = other DNA
acacctaatg

SEQ ID NO: 239 - DNA, 10 bp, synthetic construct
  source 1..10: mol_type = other DNA
accttgtgct

SEQ ID NO: 240 - DNA, 10 bp, synthetic construct
  source 1..10: mol_type = other DNA
taggtagtac

SEQ ID NO: 241 - DNA, 10 bp, synthetic construct
  source 1..10: mol_type = other DNA
caccgtcgaa

SEQ ID NO: 242 - DNA, 10 bp, synthetic construct
  source 1..10: mol_type = other DNA
ttgaaggttc

SEQ ID NO: 243 - DNA, 10 bp, synthetic construct
  source 1..10: mol_type = other DNA
gccttaacca

SEQ ID NO: 244 - DNA, 10 bp, synthetic construct
  source 1..10: mol_type = other DNA
taagcgtatc

SEQ ID NO: 245 - DNA, 10 bp, synthetic construct
  source 1..10: mol_type = other DNA
gatatgaagg

SEQ ID NO: 246 - DNA, 10 bp, synthetic construct
  source 1..10: mol_type = other DNA
agtgtccatt

SEQ ID NO: 247 - DNA, 10 bp, synthetic construct
  source 1..10: mol_type = other DNA
cctgaactgt

SEQ ID NO: 248 - DNA, 10 bp, synthetic construct
  source 1..10: mol_type = other DNA
cgatgtgcaa

SEQ ID NO: 249 - DNA, 10 bp, synthetic construct
  source 1..10: mol_type = other DNA
cgagaccagt

SEQ ID NO: 250 - DNA, 10 bp, synthetic construct
  source 1..10: mol_type = other DNA
attagaggac

SEQ ID NO: 251 - DNA, 10 bp, synthetic construct
  source 1..10: mol_type = other DNA
tacctgacgc

SEQ ID NO: 252 - DNA, 10 bp, synthetic construct
  source 1..10: mol_type = other DNA
ccgtcatcaa

SEQ ID NO: 253 - DNA, 10 bp, synthetic construct
  source 1..10: mol_type = other DNA
ggactcatat

SEQ ID NO: 254 - DNA, 10 bp, synthetic construct
  source 1..10: mol_type = other DNA
attcttggtg

SEQ ID NO: 255 - DNA, 10 bp, synthetic construct
  source 1..10: mol_type = other DNA
aacaactcca

SEQ ID NO: 256 - DNA, 10 bp, synthetic construct
  source 1..10: mol_type = other DNA
cttgcacatt

SEQ ID NO: 257 - DNA, 10 bp, synthetic construct
  source 1..10: mol_type = other DNA
tgcttggtca

SEQ ID NO: 258 - DNA, 10 bp, synthetic construct
  source 1..10: mol_type = other DNA
gctcattcat

SEQ ID NO: 259 - DNA, 10 bp, synthetic construct
  source 1..10: mol_type = other DNA
ctcacaactt

SEQ ID NO: 260 - DNA, 10 bp, synthetic construct
  source 1..10: mol_type = other DNA
tcaggccatg

SEQ ID NO: 261 - DNA, 10 bp, synthetic construct
  source 1..10: mol_type = other DNA
taccattgga

SEQ ID NO: 262 - DNA, 10 bp, synthetic construct
  source 1..10: mol_type = other DNA
aagttagctc

SEQ ID NO: 263 - DNA, 10 bp, synthetic construct
  source 1..10: mol_type = other DNA
gttaggatac

SEQ ID NO: 264 - DNA, 10 bp, synthetic construct
  source 1..10: mol_type = other DNA
aggagccaca

SEQ ID NO: 265 - DNA, 10 bp, synthetic construct
  source 1..10: mol_type = other DNA
tgacgcacaa

SEQ ID NO: 266 - DNA, 10 bp, synthetic construct
  source 1..10: mol_type = other DNA
ccttacgatt

SEQ ID NO: 267 - DNA, 10 bp, synthetic construct
  source 1..10: mol_type = other DNA
attgtactcc

SEQ ID NO: 268 - DNA, 10 bp, synthetic construct
  source 1..10: mol_type = other DNA
taggactcga

SEQ ID NO: 269 - DNA, 10 bp, synthetic construct
  source 1..10: mol_type = other DNA
tccagttgcg

SEQ ID NO: 270 - DNA, 10 bp, synthetic construct
  source 1..10: mol_type = other DNA
ggaatgaact

SEQ ID NO: 271 - DNA, 10 bp, synthetic construct
  source 1..10: mol_type = other DNA
gaaccaagaa

SEQ ID NO: 272 - DNA, 10 bp, synthetic construct
  source 1..10: mol_type = other DNA
ctcttccatt

SEQ ID NO: 273 - DNA, 10 bp, synthetic construct
  source 1..10: mol_type = other DNA
ggatacgcaa

SEQ ID NO: 274 - DNA, 10 bp, synthetic construct
  source 1..10: mol_type = other DNA
aaccgtcatt

SEQ ID NO: 275 - DNA, 10 bp, synthetic construct
  source 1..10: mol_type = other DNA
tcgtcttgcc

SEQ ID NO: 276 - DNA, 10 bp, synthetic construct
  source 1..10: mol_type = other DNA
agagtgcgca

SEQ ID NO: 277 - DNA, 10 bp, synthetic construct
  source 1..10: mol_type = other DNA
gtcttaatgg

SEQ ID NO: 278 - DNA, 10 bp, synthetic construct
  source 1..10: mol_type = other DNA
cttaccttct

SEQ ID NO: 279 - DNA, 10 bp, synthetic construct
  source 1..10: mol_type = other DNA
gagttagaag

SEQ ID NO: 280 - DNA, 10 bp, synthetic construct
  source 1..10: mol_type = other DNA
caacgaccta

SEQ ID NO: 281 - DNA, 10 bp, synthetic construct
  source 1..10: mol_type = other DNA
ggtattggaa

SEQ ID NO: 282 - DNA, 10 bp, synthetic construct
  source 1..10: mol_type = other DNA
caagcgaatt

SEQ ID NO: 283 - DNA, 10 bp, synthetic construct
  source 1..10: mol_type = other DNA
tggtgccact

SEQ ID NO: 284 - DNA, 10 bp, synthetic construct
  source 1..10: mol_type = other DNA
cttgtgttga

SEQ ID NO: 285 - DNA, 10 bp, synthetic construct
  source 1..10: mol_type = other DNA
atgcaacgtt

SEQ ID NO: 286 - DNA, 10 bp, synthetic construct
  source 1..10: mol_type = other DNA
tcgcatgcac

SEQ ID NO: 287 - DNA, 10 bp, synthetic construct
  source 1..10: mol_type = other DNA
ttcagtgtgg

SEQ ID NO: 288 - DNA, 10 bp, synthetic construct
  source 1..10: mol_type = other DNA
gaatgatcac

SEQ ID NO: 289 - DNA, 10 bp, synthetic construct
  source 1..10: mol_type = other DNA
ttgagactga

SEQ ID NO: 290 - DNA, 10 bp, synthetic construct
  source 1..10: mol_type = other DNA
gcttatgtct

SEQ ID NO: 291 - DNA, 10 bp, synthetic construct
  source 1..10: mol_type = other DNA
gaagccaggt

SEQ ID NO: 292 - DNA, 10 bp, synthetic construct
  source 1..10: mol_type = other DNA
taccagccac

SEQ ID NO: 293 - DNA, 10 bp, synthetic construct
  source 1..10: mol_type = other DNA
cgaatctgtt

SEQ ID NO: 294 - DNA, 10 bp, synthetic construct
  source 1..10: mol_type = other DNA
atagcacact

SEQ ID NO: 295 - DNA, 10 bp, synthetic construct
  source 1..10: mol_type = other DNA
ttgctagcag

SEQ ID NO: 296 - DNA, 10 bp, synthetic construct
  source 1..10: mol_type = other DNA
cggtcttatt

SEQ ID NO: 297 - DNA, 10 bp, synthetic construct
  source 1..10: mol_type = other DNA
atgttcgcaa

SEQ ID NO: 298 - DNA, 10 bp, synthetic construct
  source 1..10: mol_type = other DNA
catgacaacc

SEQ ID NO: 299 - DNA, 10 bp, synthetic construct
  source 1..10: mol_type = other DNA
agacttctgg

SEQ ID NO: 300 - DNA, 10 bp, synthetic construct
  source 1..10: mol_type = other DNA
tccatctgta

SEQ ID NO: 301 - DNA, 10 bp, synthetic construct
  source 1..10: mol_type = other DNA
gaacggttat

SEQ ID NO: 302 - DNA, 10 bp, synthetic construct
  source 1..10: mol_type = other DNA
tgtcaagttc

SEQ ID NO: 303 - DNA, 10 bp, synthetic construct
  source 1..10: mol_type = other DNA
ccgtcaagga

SEQ ID NO: 304 - DNA, 10 bp, synthetic construct
  source 1..10: mol_type = other DNA
tggaggtcct

SEQ ID NO: 305 - DNA, 10 bp, synthetic construct
  source 1..10: mol_type = other DNA
ttccaagcaa

SEQ ID NO: 306 - DNA, 10 bp, synthetic construct
  source 1..10: mol_type = other DNA
cagaggtatt

SEQ ID NO: 307 - DNA, 10 bp, synthetic construct
  source 1..10: mol_type = other DNA
agatctagca

SEQ ID NO: 308 - DNA, 10 bp, synthetic construct
  source 1..10: mol_type = other DNA
aaggtactgt

SEQ ID NO: 309 - DNA, 10 bp, synthetic construct
  source 1..10: mol_type = other DNA
gcacttggtc

SEQ ID NO: 310 - DNA, 10 bp, synthetic construct
  source 1..10: mol_type = other DNA
cgtatcctgg

SEQ ID NO: 311 - DNA, 10 bp, synthetic construct
  source 1..10: mol_type = other DNA
gtagaatcac

SEQ ID NO: 312 - DNA, 10 bp, synthetic construct
  source 1..10: mol_type = other DNA
cctaggaact

SEQ ID NO: 313 - DNA, 10 bp, synthetic construct
  source 1..10: mol_type = other DNA
ttacagcgtt

SEQ ID NO: 314 - DNA, 10 bp, synthetic construct
  source 1..10: mol_type = other DNA
cgctgtaagc

SEQ ID NO: 315 - DNA, 10 bp, synthetic construct
  source 1..10: mol_type = other DNA
gaggccatat

SEQ ID NO: 316 - DNA, 10 bp, synthetic construct
  source 1..10: mol_type = other DNA
attggaaccg

SEQ ID NO: 317 - DNA, 10 bp, synthetic construct
  source 1..10: mol_type = other DNA
gctatagcta

SEQ ID NO: 318 - DNA, 10 bp, synthetic construct
  source 1..10: mol_type = other DNA
agaggttcgt

SEQ ID NO: 319 - DNA, 10 bp, synthetic construct
  source 1..10: mol_type = other DNA
aacctcttca

SEQ ID NO: 320 - DNA, 10 bp, synthetic construct
  source 1..10: mol_type = other DNA
ccataagagt

SEQ ID NO: 321 - DNA, 10 bp, synthetic construct
  source 1..10: mol_type = other DNA
aggtgttgat

SEQ ID NO: 322 - DNA, 10 bp, synthetic construct
  source 1..10: mol_type = other DNA
tccgaggatc

SEQ ID NO: 323 - DNA, 10 bp, synthetic construct
  source 1..10: mol_type = other DNA
gaagcactat

SEQ ID NO: 324 - DNA, 10 bp, synthetic construct
  source 1..10: mol_type = other DNA
attgcagcca

SEQ ID NO: 325 - DNA, 10 bp, synthetic construct
  source 1..10: mol_type = other DNA
cggattaaga

SEQ ID NO: 326 - DNA, 10 bp, synthetic construct
  source 1..10: mol_type = other DNA
ctactacaga

SEQ ID NO: 327 - DNA, 10 bp, synthetic construct
  source 1..10: mol_type = other DNA
ggtcacatgg

SEQ ID NO: 328 - DNA, 10 bp, synthetic construct
  source 1..10: mol_type = other DNA
gcgatctcac

SEQ ID NO: 329 - DNA, 10 bp, synthetic construct
  source 1..10: mol_type = other DNA
gtcatgtcgt

SEQ ID NO: 330 - DNA, 10 bp, synthetic construct
  source 1..10: mol_type = other DNA
tggcttatcc

SEQ ID NO: 331 - DNA, 10 bp, synthetic construct
  source 1..10: mol_type = other DNA
tattgagcgt

SEQ ID NO: 332 - DNA, 10 bp, synthetic construct
  source 1..10: mol_type = other DNA
atcgccagaa

SEQ ID NO: 333 - DNA, 10 bp, synthetic construct
  source 1..10: mol_type = other DNA
cctcaacatt

SEQ ID NO: 334 - DNA, 10 bp, synthetic construct
  source 1..10: mol_type = other DNA
taagtcagtg

SEQ ID NO: 335 - DNA, 10 bp, synthetic construct
  source 1..10: mol_type = other DNA
cgctaggtat

SEQ ID NO: 336 - DNA, 10 bp, synthetic construct
  source 1..10: mol_type = other DNA
ccatgcctca

SEQ ID NO: 337 - DNA, 10 bp, synthetic construct
  source 1..10: mol_type = other DNA
caggaattga

SEQ ID NO: 338 - DNA, 10 bp, synthetic construct
  source 1..10: mol_type = other DNA
tcaatgcaac

SEQ ID NO: 339 - DNA, 10 bp, synthetic construct
  source 1..10: mol_type = other DNA
agacctatct

SEQ ID NO: 340 - DNA, 10 bp, synthetic construct
  source 1..10: mol_type = other DNA
tattccgatc

SEQ ID NO: 341 - DNA, 10 bp, synthetic construct
  source 1..10: mol_type = other DNA
gtccgtaaga

SEQ ID NO: 342 - DNA, 10 bp, synthetic construct
  source 1..10: mol_type = other DNA
tcttgtccaa

SEQ ID NO: 343 - DNA, 10 bp, synthetic construct
  source 1..10: mol_type = other DNA
gtaacgagct

SEQ ID NO: 344 - DNA, 10 bp, synthetic construct
  source 1..10: mol_type = other DNA
tggtgcttgg

SEQ ID NO: 345 - DNA, 10 bp, synthetic construct
  source 1..10: mol_type = other DNA
tgccacaatt

SEQ ID NO: 346 - DNA, 10 bp, synthetic construct
  source 1..10: mol_type = other DNA
gcttctatga

SEQ ID NO: 347 - DNA, 10 bp, synthetic construct
  source 1..10: mol_type = other DNA
caattgttcc

SEQ ID NO: 348 - DNA, 10 bp, synthetic construct
  source 1..10: mol_type = other DNA
gttgcctaga

SEQ ID NO: 349 - DNA, 10 bp, synthetic construct
  source 1..10: mol_type = other DNA
tatcaagcgg

SEQ ID NO: 350 - DNA, 10 bp, synthetic construct
  source 1..10: mol_type = other DNA
attagtcgtc

SEQ ID NO: 351 - DNA, 10 bp, synthetic construct
  source 1..10: mol_type = other DNA
ggtctaacat

SEQ ID NO: 352 - DNA, 10 bp, synthetic construct
  source 1..10: mol_type = other DNA
tggcagtaat

SEQ ID NO: 353 - DNA, 10 bp, synthetic construct
  source 1..10: mol_type = other DNA
ctctagtgat

SEQ ID NO: 354 - DNA, 10 bp, synthetic construct
  source 1..10: mol_type = other DNA
tagcttgacc

SEQ ID NO: 355 - DNA, 10 bp, synthetic construct
  source 1..10: mol_type = other DNA
ggagcaactg

SEQ ID NO: 356 - DNA, 10 bp, synthetic construct
  source 1..10: mol_type = other DNA
tatcacctca

SEQ ID NO: 357 - DNA, 10 bp, synthetic construct
  source 1..10: mol_type = other DNA
acggaacagg

SEQ ID NO: 358 - DNA, 10 bp, synthetic construct
  source 1..10: mol_type = other DNA
gctcgaggta

SEQ ID NO: 359 - DNA, 10 bp, synthetic construct
  source 1..10: mol_type = other DNA
ctgattagcc

SEQ ID NO: 360 - DNA, 10 bp, synthetic construct
  source 1..10: mol_type = other DNA
cactgcgcaa

SEQ ID NO: 361 - DNA, 10 bp, synthetic construct
  source 1..10: mol_type = other DNA
ggcatctatt

SEQ ID NO: 362 - DNA, 10 bp, synthetic construct
  source 1..10: mol_type = other DNA
ctatgaggaa

SEQ ID NO: 363 - DNA, 10 bp, synthetic construct
  source 1..10: mol_type = other DNA
atgtctacca

SEQ ID NO: 364 - DNA, 10 bp, synthetic construct
  source 1..10: mol_type = other DNA
tgttaggtga

SEQ ID NO: 365 - DNA, 10 bp, synthetic construct
  source 1..10: mol_type = other DNA
cctgatcgca

SEQ ID NO: 366 - DNA, 10 bp, synthetic construct
  source 1..10: mol_type = other DNA
aacaagaacg

SEQ ID NO: 367 - DNA, 10 bp, synthetic construct
  source 1..10: mol_type = other DNA
taccgaacac

SEQ ID NO: 368 - DNA, 10 bp, synthetic construct
  source 1..10: mol_type = other DNA
cattgcttgc

SEQ ID NO: 369 - DNA, 10 bp, synthetic construct
  source 1..10: mol_type = other DNA
cgattgagtt

SEQ ID NO: 370 - DNA, 10 bp, synthetic construct
  source 1..10: mol_type = other DNA
gagcatgcaa

SEQ ID NO: 371 - DNA, 10 bp, synthetic construct
  source 1..10: mol_type = other DNA
tcaggttagc

SEQ ID NO: 372 - DNA, 10 bp, synthetic construct
  source 1..10: mol_type = other DNA
ggttcaatca

SEQ ID NO: 373 - DNA, 10 bp, synthetic construct
  source 1..10: mol_type = other DNA
agcaagctgc

SEQ ID NO: 374 - DNA, 10 bp, synthetic construct
  source 1..10: mol_type = other DNA
gaacgctgtc

SEQ ID NO: 375 - DNA, 10 bp, synthetic construct
  source 1..10: mol_type = other DNA
attcgtcatg

SEQ ID NO: 376 - DNA, 10 bp, synthetic construct
  source 1..10: mol_type = other DNA
cagatcctaa

SEQ ID NO: 377 - DNA, 10 bp, synthetic construct
  source 1..10: mol_type = other DNA
tgcatcctga

SEQ ID NO: 378 - DNA, 10 bp, synthetic construct
  source 1..10: mol_type = other DNA
gaggagaatt

SEQ ID NO: 379 - DNA, 10 bp, synthetic construct
  source 1..10: mol_type = other DNA
tctcgatgaa

SEQ ID NO: 380 - DNA, 10 bp, synthetic construct
  source 1..10: mol_type = other DNA
agcgcataac

SEQ ID NO: 381 - DNA, 10 bp, synthetic construct
  source 1..10: mol_type = other DNA
ccaattacca

SEQ ID NO: 382 - DNA, 10 bp, synthetic construct
  source 1..10: mol_type = other DNA
agttccggaa

SEQ ID NO: 383 - DNA, 10 bp, synthetic construct
  source 1..10: mol_type = other DNA
ttaaggaagc

SEQ ID NO: 384 - DNA, 10 bp, synthetic construct
  source 1..10: mol_type = other DNA
cgcaatgtgg

SEQ ID NO: 385 - DNA, 10 bp, synthetic construct
  source 1..10: mol_type = other DNA
taagaaggcc

SEQ ID NO: 386 - DNA, 10 bp, synthetic construct
  source 1..10: mol_type = other DNA
atgtgttcgt

SEQ ID NO: 387 - DNA, 10 bp, synthetic construct
  source 1..10: mol_type = other DNA
gttcaccact

SEQ ID NO: 388 - DNA, 10 bp, synthetic construct
  source 1..10: mol_type = other DNA
ccgacgatga

SEQ ID NO: 389 - DNA, 10 bp, synthetic construct
  source 1..10: mol_type = other DNA
agaatagagg

SEQ ID NO: 390 - DNA, 10 bp, synthetic construct
  source 1..10: mol_type = other DNA
ctcattgtca

SEQ ID NO: 391 - DNA, 10 bp, synthetic construct
  source 1..10: mol_type = other DNA
gcattcgcta

SEQ ID NO: 392 - DNA, 10 bp, synthetic construct
  source 1..10: mol_type = other DNA
tgtgagctat

SEQ ID NO: 393 - DNA, 10 bp, synthetic construct
  source 1..10: mol_type = other DNA
gaacataggt

SEQ ID NO: 394 - DNA, 10 bp, synthetic construct
  source 1..10: mol_type = other DNA
tggttggata

SEQ ID NO: 395 - DNA, 10 bp, synthetic construct
  source 1..10: mol_type = other DNA
aacacctggt

SEQ ID NO: 396 - DNA, 10 bp, synthetic construct
  source 1..10: mol_type = other DNA
tatcgattcg

SEQ ID NO: 397 - DNA, 10 bp, synthetic construct
  source 1..10: mol_type = other DNA
tcattacagc

SEQ ID NO: 398 - DNA, 10 bp, synthetic construct
  source 1..10: mol_type = other DNA
ttagagctca

SEQ ID NO: 399 - DNA, 10 bp, synthetic construct
  source 1..10: mol_type = other DNA
ttggtgacaa

SEQ ID NO: 400 - DNA, 10 bp, synthetic construct
  source 1..10: mol_type = other DNA
ctgtgatatc

SEQ ID NO: 401 - DNA, 10 bp, synthetic construct
  source 1..10: mol_type = other DNA
caagtggtct

SEQ ID NO: 402 - DNA, 10 bp, synthetic construct
  source 1..10: mol_type = other DNA
atgactagga

SEQ ID NO: 403 - DNA, 10 bp, synthetic construct
  source 1..10: mol_type = other DNA
tactgtcgta

SEQ ID NO: 404 - DNA, 10 bp, synthetic construct
  source 1..10: mol_type = other DNA
acacacaagg

SEQ ID NO: 405 - DNA, 10 bp, synthetic construct
  source 1..10: mol_type = other DNA
tgcctagcgt

SEQ ID NO: 406 - DNA, 10 bp, synthetic construct
  source 1..10: mol_type = other DNA
gctatcctct

SEQ ID NO: 407 - DNA, 10 bp, synthetic construct
  source 1..10: mol_type = other DNA
tgctagttgt

SEQ ID NO: 408 - DNA, 10 bp, synthetic construct
  source 1..10: mol_type = other DNA
gtacacggac

SEQ ID NO: 409 - DNA, 10 bp, synthetic construct
  source 1..10: mol_type = other DNA
accttgtgtt

SEQ ID NO: 410 - DNA, 10 bp, synthetic construct
  source 1..10: mol_type = other DNA
ggaagtgacc

SEQ ID NO: 411 - DNA, 10 bp, synthetic construct
  source 1..10: mol_type = other DNA
tatcctctgg

SEQ ID NO: 412 - DNA, 10 bp, synthetic construct
  source 1..10: mol_type = other DNA
ctagaccaat

SEQ ID NO: 413 - DNA, 10 bp, synthetic construct
  source 1..10: mol_type = other DNA
agcagtacta

SEQ ID NO: 414 - DNA, 10 bp, synthetic construct
  source 1..10: mol_type = other DNA
aagccatgag

SEQ ID NO: 415 - DNA, 10 bp, synthetic construct
  source 1..10: mol_type = other DNA
tcttagccga

SEQ ID NO: 416 - DNA, 10 bp, synthetic construct
  source 1..10: mol_type = other DNA
gcgataatac

SEQ ID NO: 417 - DNA, 10 bp, synthetic construct
  source 1..10: mol_type = other DNA
agaggcatgg

SEQ ID NO: 418 - DNA, 10 bp, synthetic construct
  source 1..10: mol_type = other DNA
ttctatcctc

SEQ ID NO: 419 - DNA, 10 bp, synthetic construct
  source 1..10: mol_type = other DNA
tgtctggact

SEQ ID NO: 420 - DNA, 10 bp, synthetic construct
  source 1..10: mol_type = other DNA
cagcggtgaa

SEQ ID NO: 421 - DNA, 10 bp, synthetic construct
  source 1..10: mol_type = other DNA
acaactacag

SEQ ID NO: 422 - DNA, 10 bp, synthetic construct
  source 1..10: mol_type = other DNA
gaggaagtaa

SEQ ID NO: 423 - DNA, 10 bp, synthetic construct
  source 1..10: mol_type = other DNA
attgtcagct

SEQ ID NO: 424 - DNA, 10 bp, synthetic construct
  source 1..10: mol_type = other DNA
tcgccagatc

SEQ ID NO: 425 - DNA, 10 bp, synthetic construct
  source 1..10: mol_type = other DNA
tcgacacaga

SEQ ID NO: 426 - DNA, 10 bp, synthetic construct
  source 1..10: mol_type = other DNA
gaatccacag

SEQ ID NO: 427 - DNA, 10 bp, synthetic construct
  source 1..10: mol_type = other DNA
cctcttgatt

SEQ ID NO: 428 - DNA, 10 bp, synthetic construct
  source 1..10: mol_type = other DNA
atctggaggt

SEQ ID NO: 429 - DNA, 10 bp, synthetic construct
  source 1..10: mol_type = other DNA
ggtgagctca

SEQ ID NO: 430 - DNA, 10 bp, synthetic construct
  source 1..10: mol_type = other DNA
ctccaattcc

SEQ ID NO: 431 - DNA, 10 bp, synthetic construct
  source 1..10: mol_type = other DNA
tgacgtcaat

SEQ ID NO: 432 - DNA, 10 bp, synthetic construct
  source 1..10: mol_type = other DNA
aagaatggac

SEQ ID NO: 433 - DNA, 10 bp, synthetic construct
  source 1..10: mol_type = other DNA
attccacaca

SEQ ID NO: 434 - DNA, 10 bp, synthetic construct
  source 1..10: mol_type = other DNA
tggttctgaa

SEQ ID NO: 435 - DNA, 10 bp, synthetic construct
  source 1..10: mol_type = other DNA
tacaatgctc

SEQ ID NO: 436 - DNA, 10 bp, synthetic construct
  source 1..10: mol_type = other DNA
gtagtggtgc

SEQ ID NO: 437 - DNA, 10 bp, synthetic construct
  source 1..10: mol_type = other DNA
cgatccattg

SEQ ID NO: 438 - DNA, 10 bp, synthetic construct
  source 1..10: mol_type = other DNA
acaagactgt

SEQ ID NO: 439 - DNA, 10 bp, synthetic construct
  source 1..10: mol_type = other DNA
cgcggttcat

SEQ ID NO: 440 - DNA, 10 bp, synthetic construct
  source 1..10: mol_type = other DNA
attcggaaca

SEQ ID NO: 441 - DNA, 10 bp, synthetic construct
  source 1..10: mol_type = other DNA
acacaagaac

SEQ ID NO: 442 - DNA, 10 bp, synthetic construct
  source 1..10: mol_type = other DNA
gttaccagcg

SEQ ID NO: 443 - DNA, 10 bp, synthetic construct
  source 1..10: mol_type = other DNA
gagttggctc

SEQ ID NO: 444 - DNA, 10 bp, synthetic construct
  source 1..10: mol_type = other DNA
ttcggttagt

SEQ ID NO: 445 - DNA, 10 bp, synthetic construct
  source 1..10: mol_type = other DNA
cgagcctcaa

SEQ ID NO: 446 - DNA, 10 bp, synthetic construct
  source 1..10: mol_type = other DNA
agcaggctca

SEQ ID NO: 447 - DNA, 10 bp, synthetic construct
  source 1..10: mol_type = other DNA
cagtatagag

SEQ ID NO: 448 - DNA, 10 bp, synthetic construct
  source 1..10: mol_type = other DNA
ggttcaccgt

SEQ ID NO: 449 - DNA, 10 bp, synthetic construct
  source 1..10: mol_type = other DNA
tgttcgttcg

SEQ ID NO: 450 - DNA, 10 bp, synthetic construct
  source 1..10: mol_type = other DNA
acagtccatt

SEQ ID NO: 451 - DNA, 10 bp, synthetic construct
  source 1..10: mol_type = other DNA
ctgtgaagat

SEQ ID NO: 452 - DNA, 10 bp, synthetic construct
  source 1..10: mol_type = other DNA
gctattctga

SEQ ID NO: 453 - DNA, 10 bp, synthetic construct
  source 1..10: mol_type = other DNA
gacgaggaat

SEQ ID NO: 454 - DNA, 10 bp, synthetic construct
  source 1..10: mol_type = other DNA
ttgacagcgc

SEQ ID NO: 455 - DNA, 10 bp, synthetic construct
  source 1..10: mol_type = other DNA
agccgatgtc

SEQ ID NO: 456 - DNA, 10 bp, synthetic construct
  source 1..10: mol_type = other DNA
taacacagtg

SEQ ID NO: 457 - DNA, 10 bp, synthetic construct
  source 1..10: mol_type = other DNA
actagcggac

SEQ ID NO: 458 - DNA, 10 bp, synthetic construct
  source 1..10: mol_type = other DNA
ttagcaacgt

SEQ ID NO: 459 - DNA, 10 bp, synthetic construct
  source 1..10: mol_type = other DNA
cacaatatcg

SEQ ID NO: 460 - DNA, 10 bp, synthetic construct
  source 1..10: mol_type = other DNA
acctggtata

SEQ ID NO: 461 - DNA, 10 bp, synthetic construct
  source 1..10: mol_type = other DNA
acacttcttc

SEQ ID NO: 462 - DNA, 10 bp, synthetic construct
  source 1..10: mol_type = other DNA
tggtcagcag

SEQ ID NO: 463 - DNA, 10 bp, synthetic construct
  source 1..10: mol_type = other DNA
gtattccaga

SEQ ID NO: 464 - DNA, 10 bp, synthetic construct
  source 1..10: mol_type = other DNA
cgtgactgct

SEQ ID NO: 465 - DNA, 10 bp, synthetic construct
  source 1..10: mol_type = other DNA
aagcctccat

SEQ ID NO: 466 - DNA, 10 bp, synthetic construct
  source 1..10: mol_type = other DNA
atctgaatgc

SEQ ID NO: 467 - DNA, 10 bp, synthetic construct
  source 1..10: mol_type = other DNA
ggtaacagag

SEQ ID NO: 468 - DNA, 10 bp, synthetic construct
  source 1..10: mol_type = other DNA
ccgacttcca

SEQ ID NO: 469 - DNA, 10 bp, synthetic construct
  source 1..10: mol_type = other DNA
tcagtggaac

SEQ ID NO: 470 - DNA, 10 bp, synthetic construct
  source 1..10: mol_type = other DNA
tgtctctgtt

SEQ ID NO: 471 - DNA, 10 bp, synthetic construct
  source 1..10: mol_type = other DNA
aattaccgga

SEQ ID NO: 472 - DNA, 10 bp, synthetic construct
  source 1..10: mol_type = other DNA
gtcataatcg

SEQ ID NO: 473 - DNA, 10 bp, synthetic construct
  source 1..10: mol_type = other DNA
ttaagctgga

SEQ ID NO: 474 - DNA, 10 bp, synthetic construct
  source 1..10: mol_type = other DNA
tatgctgcac

SEQ ID NO: 475 - DNA, 10 bp, synthetic construct
  source 1..10: mol_type = other DNA
ccgataactg

SEQ ID NO: 476 - DNA, 10 bp, synthetic construct
  source 1..10: mol_type = other DNA
ggcctgcatt

SEQ ID NO: 477 - DNA, 10 bp, synthetic construct
  source 1..10: mol_type = other DNA
aagtacgtac

SEQ ID NO: 478 - DNA, 10 bp, synthetic construct
  source 1..10: mol_type = other DNA
tccttatgcc

SEQ ID NO: 479 - DNA, 10 bp, synthetic construct
  source 1..10: mol_type = other DNA
ctacggccta

SEQ ID NO: 480 - DNA, 10 bp, synthetic construct
  source 1..10: mol_type = other DNA
acctatgaga

SEQ ID NO: 481 - DNA, 10 bp, synthetic construct
  source 1..10: mol_type = other DNA
agccgcgtat

SEQ ID NO: 482 - DNA, 10 bp, synthetic construct
  source 1..10: mol_type = other DNA
ttgttgtctg

SEQ ID NO: 483 - DNA, 10 bp, synthetic construct
  source 1..10: mol_type = other DNA
ccagaacgga

SEQ ID NO: 484 - DNA, 10 bp, synthetic construct
  source 1..10: mol_type = other DNA
ggaacgatcc

SEQ ID NO: 485 - DNA, 10 bp, synthetic construct
  source 1..10: mol_type = other DNA
catcctgcag

SEQ ID NO: 486 - DNA, 10 bp, synthetic construct
  source 1..10: mol_type = other DNA
aacgcttaga

SEQ ID NO: 487 - DNA, 10 bp, synthetic construct
  source 1..10: mol_type = other DNA
ttgattgacc

SEQ ID NO: 488 - DNA, 10 bp, synthetic construct
  source 1..10: mol_type = other DNA
gccttacgtt

SEQ ID NO: 489 - DNA, 10 bp, synthetic construct
  source 1..10: mol_type = other DNA
atatcagtcc

SEQ ID NO: 490 - DNA, 10 bp, synthetic construct
  source 1..10: mol_type = other DNA
tccgacagga

SEQ ID NO: 491 - DNA, 10 bp, synthetic construct
  source 1..10: mol_type = other DNA
aagattgctg

SEQ ID NO: 492 - DNA, 10 bp, synthetic construct
  source 1..10: mol_type = other DNA
tatcggtacg

SEQ ID NO: 493 - DNA, 10 bp, synthetic construct
  source 1..10: mol_type = other DNA
cgagaactat

SEQ ID NO: 494 - DNA, 10 bp, synthetic construct
  source 1..10: mol_type = other DNA
gacagaagcg

SEQ ID NO: 495 - DNA, 10 bp, synthetic construct
  source 1..10: mol_type = other DNA
gcattgcctc

SEQ ID NO: 496 - DNA, 10 bp, synthetic construct
  source 1..10: mol_type = other DNA
agtactgaag

SEQ ID NO: 497 - DNA, 10 bp, synthetic construct
  source 1..10: mol_type = other DNA
acatcgccta

SEQ ID NO: 498 - DNA, 10 bp, synthetic construct
  source 1..10: mol_type = other DNA
ttccaagtcg

SEQ ID NO: 499 - DNA, 10 bp, synthetic construct
  source 1..10: mol_type = other DNA
aggactaatc

SEQ ID NO: 500 - DNA, 10 bp, synthetic construct
  source 1..10: mol_type = other DNA
cctggatgga

SEQ ID NO: 501 - DNA, 10 bp, synthetic construct
  source 1..10: mol_type = other DNA
gatagctgat

SEQ ID NO: 502 - DNA, 10 bp, synthetic construct
  source 1..10: mol_type = other DNA
cgcgttaggt

SEQ ID NO: 503 - DNA, 10 bp, synthetic construct
  source 1..10: mol_type = other DNA
taccagctat

SEQ ID NO: 504 - DNA, 10 bp, synthetic construct
  source 1..10: mol_type = other DNA
ctctacgtcg

SEQ ID NO: 505 - DNA, 10 bp, synthetic construct
  source 1..10: mol_type = other DNA
ttatgaggcc

SEQ ID NO: 506 - DNA, 10 bp, synthetic construct
  source 1..10: mol_type = other DNA
accaagcagg

SEQ ID NO: 507 - DNA, 10 bp, synthetic construct
  source 1..10: mol_type = other DNA
gtggacagaa

SEQ ID NO: 508 - DNA, 10 bp, synthetic construct
  source 1..10: mol_type = other DNA
cgaccttctg

SEQ ID NO: 509 - DNA, 10 bp, synthetic construct
  source 1..10: mol_type = other DNA
ttacactcgt

SEQ ID NO: 510 - DNA, 10 bp, synthetic construct
  source 1..10: mol_type = other DNA
tacgttgttc

SEQ ID NO: 511 - DNA, 10 bp, synthetic construct
  source 1..10: mol_type = other DNA
aattcaggca

SEQ ID NO: 512 - DNA, 10 bp, synthetic construct
  source 1..10: mol_type = other DNA
tcgatgctaa

SEQ ID NO: 513 - DNA, 10 bp, synthetic construct
  source 1..10: mol_type = other DNA
aattctcacg

SEQ ID NO: 514 - DNA, 10 bp, synthetic construct
  source 1..10: mol_type = other DNA
tcgcagtgac

SEQ ID NO: 515 - DNA, 10 bp, synthetic construct
  source 1..10: mol_type = other DNA
ggcagagtta

SEQ ID NO: 516 - DNA, 10 bp, synthetic construct
  source 1..10: mol_type = other DNA
ttcttaacgg

SEQ ID NO: 517 - DNA, 10 bp, synthetic construct
  source 1..10: mol_type = other DNA
caatgccaat

SEQ ID NO: 518 - DNA, 10 bp, synthetic construct
  source 1..10: mol_type = other DNA
acaggagcca

SEQ ID NO: 519 - DNA, 10 bp, synthetic construct
  source 1..10: mol_type = other DNA
cgttctactt

SEQ ID NO: 520 - DNA, 10 bp, synthetic construct
  source 1..10: mol_type = other DNA
atgcagattc

SEQ ID NO: 521 - DNA, 10 bp, synthetic construct
  source 1..10: mol_type = other DNA
tccatggctg

SEQ ID NO: 522 - DNA, 10 bp, synthetic construct
  source 1..10: mol_type = other DNA
gtaccaagaa

SEQ ID NO: 523 - DNA, 10 bp, synthetic construct
  source 1..10: mol_type = other DNA
agtggtcaat

SEQ ID NO: 524 - DNA, 10 bp, synthetic construct
  source 1..10: mol_type = other DNA
cgttacgtgg

SEQ ID NO: 525 - DNA, 10 bp, synthetic construct
  source 1..10: mol_type = other DNA
tacttacgca

SEQ ID NO: 526 - DNA, 10 bp, synthetic construct
  source 1..10: mol_type = other DNA
tggcgttaac

SEQ ID NO: 527 - DNA, 10 bp, synthetic construct
  source 1..10: mol_type = other DNA
gaagagtaca

SEQ ID NO: 528 - DNA, 10 bp, synthetic construct
  source 1..10: mol_type = other DNA
atgaccattc

SEQ ID NO: 529 - DNA, 10 bp, synthetic construct
  source 1..10: mol_type = other DNA
tggtaggaag

SEQ ID NO: 530 - DNA, 10 bp, synthetic construct
  source 1..10: mol_type = other DNA
gtagttcgga

SEQ ID NO: 531 - DNA, 10 bp, synthetic construct
  source 1..10: mol_type = other DNA
cctagtacat

SEQ ID NO: 532 - DNA, 10 bp, synthetic construct
  source 1..10: mol_type = other DNA
cactgcgtct

SEQ ID NO: 533 - DNA, 10 bp, synthetic construct
  source 1..10: mol_type = other DNA
taacaccttc

SEQ ID NO: 534 - DNA, 10 bp, synthetic construct
  source 1..10: mol_type = other DNA
acggcattct

SEQ ID NO: 535 - DNA, 10 bp, synthetic construct
  source 1..10: mol_type = other DNA
ctactgtctc

SEQ ID NO: 536 - DNA, 10 bp, synthetic construct
  source 1..10: mol_type = other DNA
aggcgtaaga

SEQ ID NO: 537 - DNA, 10 bp, synthetic construct
  source 1..10: mol_type = other DNA
aggcgtaaga

SEQ ID NO: 538 - DNA, 10 bp, synthetic construct
  source 1..10: mol_type = other DNA
agcattctcg

SEQ ID NO: 539 - DNA, 10 bp, synthetic construct
  source 1..10: mol_type = other DNA
caacgtcctc

SEQ ID NO: 540 - DNA, 10 bp, synthetic construct
  source 1..10: mol_type = other DNA
agtgtggaat

SEQ ID NO: 541 - DNA, 10 bp, synthetic construct
  source 1..10: mol_type = other DNA
gcgcaagtca

SEQ ID NO: 542 - DNA, 10 bp, synthetic construct
  source 1..10: mol_type = other DNA
ctcagctcca

SEQ ID NO: 543 - DNA, 10 bp, synthetic construct
  source 1..10: mol_type = other DNA
aacacatggt

SEQ ID NO: 544 - DNA, 10 bp, synthetic construct
  source 1..10: mol_type = other DNA
gatgcgagac

SEQ ID NO: 545 - DNA, 10 bp, synthetic construct
  source 1..10: mol_type = other DNA
aatcgaccga

SEQ ID NO: 546 - DNA, 10 bp, synthetic construct
  source 1..10: mol_type = other DNA
tcggagttcc

SEQ ID NO: 547 - DNA, 10 bp, synthetic construct
  source 1..10: mol_type = other DNA
cgatggagtg

SEQ ID NO: 548 - DNA, 10 bp, synthetic construct
  source 1..10: mol_type = other DNA
gtctcagtag

SEQ ID NO: 549 - DNA, 10 bp, synthetic construct
  source 1..10: mol_type = other DNA
tacacttgca

SEQ ID NO: 550 - DNA, 10 bp, synthetic construct
  source 1..10: mol_type = other DNA
gtaatcgacg

SEQ ID NO: 551 - DNA, 10 bp, synthetic construct
  source 1..10: mol_type = other DNA
agtgagacgt

SEQ ID NO: 552 - DNA, 10 bp, synthetic construct
  source 1..10: mol_type = other DNA
cacctcctac

SEQ ID NO: 553 - DNA, 10 bp, synthetic construct
  source 1..10: mol_type = other DNA
acttccgtcc

SEQ ID NO: 554 - DNA, 10 bp, synthetic construct
  source 1..10: mol_type = other DNA
agccagtgga

SEQ ID NO: 555 - DNA, 10 bp, synthetic construct
  source 1..10: mol_type = other DNA
cagtgatcgg

SEQ ID NO: 556 - DNA, 10 bp, synthetic construct
  source 1..10: mol_type = other DNA
gtaactaacc

SEQ ID NO: 557 - DNA, 10 bp, synthetic construct
  source 1..10: mol_type = other DNA
tgtgccttat

SEQ ID NO: 558 - DNA, 10 bp, synthetic construct
  source 1..10: mol_type = other DNA
tgaatacgtc

SEQ ID NO: 559 - DNA, 10 bp, synthetic construct
  source 1..10: mol_type = other DNA
gtcggtaagg

SEQ ID NO: 560 - DNA, 10 bp, synthetic construct
  source 1..10: mol_type = other DNA
caagtgagtt

SEQ ID NO: 561 - DNA, 10 bp, synthetic construct
  source 1..10: mol_type = other DNA
taggccgata

SEQ ID NO: 562 - DNA, 10 bp, synthetic construct
  source 1..10: mol_type = other DNA
acactgtagt

SEQ ID NO: 563 - DNA, 10 bp, synthetic construct
  source 1..10: mol_type = other DNA
tgcggtatta

SEQ ID NO: 564 - DNA, 10 bp, synthetic construct
  source 1..10: mol_type = other DNA
ttgtagtacg

SEQ ID NO: 565 - DNA, 10 bp, synthetic construct
  source 1..10: mol_type = other DNA
agtacaagtc

SEQ ID NO: 566 - DNA, 10 bp, synthetic construct
  source 1..10: mol_type = other DNA
ctgtatccat

SEQ ID NO: 567 - DNA, 10 bp, synthetic construct
  source 1..10: mol_type = other DNA
gacatcattc

SEQ ID NO: 568 - DNA, 10 bp, synthetic construct
  source 1..10: mol_type = other DNA
gttctaagag

SEQ ID NO: 569 - DNA, 10 bp, synthetic construct
  source 1..10: mol_type = other DNA
aagtcgagag

SEQ ID NO: 570 - DNA, 10 bp, synthetic construct
  source 1..10: mol_type = other DNA
tccggactca

SEQ ID NO: 571 - DNA, 10 bp, synthetic construct
  source 1..10: mol_type = other DNA
cttaacacct

SEQ ID NO: 572 - DNA, 10 bp, synthetic construct
  source 1..10: mol_type = other DNA
ggttatcttc

SEQ ID NO: 573 - DNA, 10 bp, synthetic construct
  source 1..10: mol_type = other DNA
tgagtagatc

SEQ ID NO: 574 - DNA, 10 bp, synthetic construct
  source 1..10: mol_type = other DNA
atgcggtggt

SEQ ID NO: 575 - DNA, 10 bp, synthetic construct
  source 1..10: mol_type = other DNA
taagttgcct

SEQ ID NO: 576 - DNA, 10 bp, synthetic construct
  source 1..10: mol_type = other DNA
acacaccatg

SEQ ID NO: 577 - DNA, 10 bp, synthetic construct
  source 1..10: mol_type = other DNA
aagtggtagg

SEQ ID NO: 578 - DNA, 10 bp, synthetic construct
  source 1..10: mol_type = other DNA
ttggacggac

SEQ ID NO: 579 - DNA, 10 bp, synthetic construct
  source 1..10: mol_type = other DNA
ggacttcgct

SEQ ID NO: 580 - DNA, 10 bp, synthetic construct
  source 1..10: mol_type = other DNA
gatacagcta

SEQ ID NO: 581 - DNA, 10 bp, synthetic construct
  source 1..10: mol_type = other DNA
cacaccgcat

SEQ ID NO: 582 - DNA, 10 bp, synthetic construct
  source 1..10: mol_type = other DNA
cctccgatct

SEQ ID NO: 583 - DNA, 10 bp, synthetic construct
  source 1..10: mol_type = other DNA
agttgtccta

SEQ ID NO: 584 - DNA, 10 bp, synthetic construct
  source 1..10: mol_type = other DNA
gtaggcaatg

SEQ ID NO: 585 - DNA, 10 bp, synthetic construct
  source 1..10: mol_type = other DNA
tgacaactct

SEQ ID NO: 586 - DNA, 10 bp, synthetic construct
  source 1..10: mol_type = other DNA
acgtgcaagc

SEQ ID NO: 587 - DNA, 10 bp, synthetic construct
  source 1..10: mol_type = other DNA
tatatcggag

SEQ ID NO: 588 - DNA, 10 bp, synthetic construct
  source 1..10: mol_type = other DNA
aatacttccg

SEQ ID NO: 589 - DNA, 10 bp, synthetic construct
  source 1..10: mol_type = other DNA
gtggtatcaa

SEQ ID NO: 590 - DNA, 10 bp, synthetic construct
  source 1..10: mol_type = other DNA
gtggtatcaa

SEQ ID NO: 591 - DNA, 10 bp, synthetic construct
  source 1..10: mol_type = other DNA
cgactgatgt

SEQ ID NO: 592 - DNA, 10 bp, synthetic construct
  source 1..10: mol_type = other DNA
ggctcttgtc

SEQ ID NO: 593 - DNA, 10 bp, synthetic construct
  source 1..10: mol_type = other DNA
aacctacaac

SEQ ID NO: 594 - DNA, 10 bp, synthetic construct
  source 1..10: mol_type = other DNA
tgttattgcg

SEQ ID NO: 595 - DNA, 10 bp, synthetic construct
  source 1..10: mol_type = other DNA
tacacgatca

SEQ ID NO: 596 - DNA, 10 bp, synthetic construct
  source 1..10: mol_type = other DNA
actgacgcgt

SEQ ID NO: 597 - DNA, 10 bp, synthetic construct
  source 1..10: mol_type = other DNA
gtatgcggtc

SEQ ID NO: 598 - DNA, 10 bp, synthetic construct
  source 1..10: mol_type = other DNA
cctgactacg

SEQ ID NO: 599 - DNA, 10 bp, synthetic construct
  source 1..10: mol_type = other DNA
cggatacagg

SEQ ID NO: 600 - DNA, 10 bp, synthetic construct
  source 1..10: mol_type = other DNA
atacgtagga

SEQ ID NO: 601 - DNA, 10 bp, synthetic construct
  source 1..10: mol_type = other DNA
tgtatcagac

SEQ ID NO: 602 - DNA, 10 bp, synthetic construct
  source 1..10: mol_type = other DNA
ctgccttcct

SEQ ID NO: 603 - DNA, 10 bp, synthetic construct
  source 1..10: mol_type = other DNA
accttccagg

SEQ ID NO: 604 - DNA, 10 bp, synthetic construct
  source 1..10: mol_type = other DNA
gaaggattga

SEQ ID NO: 605 - DNA, 10 bp, synthetic construct
  source 1..10: mol_type = other DNA
ataaggcttg

SEQ ID NO: 606 - DNA, 10 bp, synthetic construct
  source 1..10: mol_type = other DNA
gatcaggcct

SEQ ID NO: 607 - DNA, 10 bp, synthetic construct
  source 1..10: mol_type = other DNA
gaatgaagac

SEQ ID NO: 608 - DNA, 10 bp, synthetic construct
  source 1..10: mol_type = other DNA
acgcttgatg

SEQ ID NO: 609 - DNA, 10 bp, synthetic construct
  source 1..10: mol_type = other DNA
atcatcacgt

SEQ ID NO: 610 - DNA, 10 bp, synthetic construct
  source 1..10: mol_type = other DNA
ccaggttgac

SEQ ID NO: 611 - DNA, 10 bp, synthetic construct
  source 1..10: mol_type = other DNA
gatcagttca

SEQ ID NO: 612 - DNA, 10 bp, synthetic construct
  source 1..10: mol_type = other DNA
aggtgacatc

SEQ ID NO: 613 - DNA, 10 bp, synthetic construct
  source 1..10: mol_type = other DNA
ttctcggaag

SEQ ID NO: 614 - DNA, 10 bp, synthetic construct
  source 1..10: mol_type = other DNA
gcaccaccaa

SEQ ID NO: 615 - DNA, 10 bp, synthetic construct
  source 1..10: mol_type = other DNA
ttgatggcgg

SEQ ID NO: 616 - DNA, 10 bp, synthetic construct
  source 1..10: mol_type = other DNA
ggacattgtt

SEQ ID NO: 617 - DNA, 10 bp, synthetic construct
  source 1..10: mol_type = other DNA
acagcagatg

SEQ ID NO: 618 - DNA, 10 bp, synthetic construct
  source 1..10: mol_type = other DNA
ggccttagaa

SEQ ID NO: 619 - DNA, 10 bp, synthetic construct
  source 1..10: mol_type = other DNA
ttgaggtaac

SEQ ID NO: 620 - DNA, 10 bp, synthetic construct
  source 1..10: mol_type = other DNA
agataacgct

SEQ ID NO: 621 - DNA, 10 bp, synthetic construct
  source 1..10: mol_type = other DNA
tgttgcatgc

SEQ ID NO: 622 - DNA, 10 bp, synthetic construct
  source 1..10: mol_type = other DNA
cagcacacct

SEQ ID NO: 623 - DNA, 10 bp, synthetic construct
  source 1..10: mol_type = other DNA
gagaggtcca

SEQ ID NO: 624 - DNA, 10 bp, synthetic construct
  source 1..10: mol_type = other DNA
atggttgtgg

SEQ ID NO: 625 - DNA, 10 bp, synthetic construct
  source 1..10: mol_type = other DNA
ttcacctgct

SEQ ID NO: 626 - DNA, 10 bp, synthetic construct
  source 1..10: mol_type = other DNA
agatgtcaga

SEQ ID NO: 627 - DNA, 10 bp, synthetic construct
  source 1..10: mol_type = other DNA
taggtggctg

SEQ ID NO: 628 - DNA, 10 bp, synthetic construct
  source 1..10: mol_type = other DNA
gctaagttac

SEQ ID NO: 629 - DNA, 10 bp, synthetic construct
  source 1..10: mol_type = other DNA
cgctcgagtt

SEQ ID NO: 630 - DNA, 10 bp, synthetic construct
  source 1..10: mol_type = other DNA
cctcgaatga

SEQ ID NO: 631 - DNA, 10 bp, synthetic construct
  source 1..10: mol_type = other DNA
ataatctcgg

SEQ ID NO: 632 - DNA, 10 bp, synthetic construct
  source 1..10: mol_type = other DNA
tagaacgaac

SEQ ID NO: 633 - DNA, 10 bp, synthetic construct
  source 1..10: mol_type = other DNA
tcactaacga

SEQ ID NO: 634 - DNA, 10 bp, synthetic construct
  source 1..10: mol_type = other DNA
atcacttgac

SEQ ID NO: 635 - DNA, 10 bp, synthetic construct
  source 1..10: mol_type = other DNA
ggtgaccagt

SEQ ID NO: 636 - DNA, 10 bp, synthetic construct
  source 1..10: mol_type = other DNA
gatcgtattc

SEQ ID NO: 637 - DNA, 10 bp, synthetic construct
  source 1..10: mol_type = other DNA
ttgtctgatg

SEQ ID NO: 638 - DNA, 10 bp, synthetic construct
  source 1..10: mol_type = other DNA
catacggacc

SEQ ID NO: 639 - DNA, 10 bp, synthetic construct
  source 1..10: mol_type = other DNA
gccggtctat

SEQ ID NO: 640 - DNA, 10 bp, synthetic construct
  source 1..10: mol_type = other DNA
agaataccga

SEQ ID NO: 641 - DNA, 10 bp, synthetic construct
  source 1..10: mol_type = other DNA
agtaagatgg

SEQ ID NO: 642 - DNA, 10 bp, synthetic construct
  source 1..10: mol_type = other DNA
tgaggaccaa

SEQ ID NO: 643 - DNA, 10 bp, synthetic construct
  source 1..10: mol_type = other DNA
ctgtaatgtc

SEQ ID NO: 644 - DNA, 10 bp, synthetic construct
  source 1..10: mol_type = other DNA
gaccttgact

SEQ ID NO: 645 - DNA, 10 bp, synthetic construct
  source 1..10: mol_type = other DNA
acctactcta

SEQ ID NO: 646 - DNA, 10 bp, synthetic construct
  source 1..10: mol_type = other DNA
ccaacgtata

SEQ ID NO: 647 - DNA, 10 bp, synthetic construct
  source 1..10: mol_type = other DNA
ttagttcacg

SEQ ID NO: 648 - DNA, 10 bp, synthetic construct
  source 1..10: mol_type = other DNA
tgttccgagc

SEQ ID NO: 649 - DNA, 10 bp, synthetic construct
  source 1..10: mol_type = other DNA
atgttgacgt

SEQ ID NO: 650 - DNA, 10 bp, synthetic construct
  source 1..10: mol_type = other DNA
gcaagataac

SEQ ID NO: 651 - DNA, 10 bp, synthetic construct
  source 1..10: mol_type = other DNA
taggcaggag

SEQ ID NO: 652 - DNA, 10 bp, synthetic construct
  source 1..10: mol_type = other DNA
cctcattctt

SEQ ID NO: 653 - DNA, 10 bp, synthetic construct
  source 1..10: mol_type = other DNA
agcaaccgca

SEQ ID NO: 654 - DNA, 10 bp, synthetic construct
  source 1..10: mol_type = other DNA
gatgtctatc

SEQ ID NO: 655 - DNA, 10 bp, synthetic construct
  source 1..10: mol_type = other DNA
tgaacgctta

SEQ ID NO: 656 - DNA, 10 bp, synthetic construct
  source 1..10: mol_type = other DNA
tagcgcgaag

SEQ ID NO: 657 - DNA, 10 bp, synthetic construct
  source 1..10: mol_type = other DNA
agtcgaagcc

SEQ ID NO: 658 - DNA, 10 bp, synthetic construct
  source 1..10: mol_type = other DNA
tcgtcgccaa

SEQ ID NO: 659 - DNA, 10 bp, synthetic construct
  source 1..10: mol_type = other DNA
atcgctgtct

SEQ ID NO: 660 - DNA, 10 bp, synthetic construct
  source 1..10: mol_type = other DNA
tgcaaccatt

SEQ ID NO: 661 - DNA, 10 bp, synthetic construct
  source 1..10: mol_type = other DNA
caggagttgg

SEQ ID NO: 662 - DNA, 10 bp, synthetic construct
  source 1..10: mol_type = other DNA
tgagtctcgc

SEQ ID NO: 663 - DNA, 10 bp, synthetic construct
  source 1..10: mol_type = other DNA
gttacaacac

SEQ ID NO: 664 - DNA, 10 bp, synthetic construct
  source 1..10: mol_type = other DNA
gcaatagatg

SEQ ID NO: 665 - DNA, 10 bp, synthetic construct
  source 1..10: mol_type = other DNA
aagagcctga

SEQ ID NO: 666 - DNA, 10 bp, synthetic construct
  source 1..10: mol_type = other DNA
gaacaagccg

SEQ ID NO: 667 - DNA, 10 bp, synthetic construct
  source 1..10: mol_type = other DNA
cgcacttatt

SEQ ID NO: 668 - DNA, 10 bp, synthetic construct
  source 1..10: mol_type = other DNA
tgcatgacgc

SEQ ID NO: 669 - DNA, 10 bp, synthetic construct
  source 1..10: mol_type = other DNA
gctgaaggaa

SEQ ID NO: 670 - DNA, 10 bp, synthetic construct
  source 1..10: mol_type = other DNA
gttgacgtta

SEQ ID NO: 671 - DNA, 10 bp, synthetic construct
  source 1..10: mol_type = other DNA
ctctattgag

SEQ ID NO: 672 - DNA, 10 bp, synthetic construct
  source 1..10: mol_type = other DNA
aagtgccgtc

SEQ ID NO: 673 - DNA, 10 bp, synthetic construct
  source 1..10: mol_type = other DNA
acgacaacca

SEQ ID NO: 674 - DNA, 10 bp, synthetic construct
  source 1..10: mol_type = other DNA
ctattgctac

SEQ ID NO: 675 - DNA, 10 bp, synthetic construct
  source 1..10: mol_type = other DNA
tcgttctcgg

SEQ ID NO: 676 - DNA, 10 bp, synthetic construct
  source 1..10: mol_type = other DNA
gatgataggt

SEQ ID NO: 677 - DNA, 10 bp, synthetic construct
  source 1..10: mol_type = other DNA
tacaaggaga

SEQ ID NO: 678 - DNA, 10 bp, synthetic construct
  source 1..10: mol_type = other DNA
gtccgcagtt

SEQ ID NO: 679 - DNA, 10 bp, synthetic construct
  source 1..10: mol_type = other DNA
agagtatgct

SEQ ID NO: 680 - DNA, 10 bp, synthetic construct
  source 1..10: mol_type = other DNA
cgaccggtta

SEQ ID NO: 681 - DNA, 10 bp, synthetic construct
  source 1..10: mol_type = other DNA
atatggacga

SEQ ID NO: 682 - DNA, 10 bp, synthetic construct
  source 1..10: mol_type = other DNA
agaattgtcc

SEQ ID NO: 683 - DNA, 10 bp, synthetic construct
  source 1..10: mol_type = other DNA
tatgttccag

SEQ ID NO: 684 - DNA, 10 bp, synthetic construct
  source 1..10: mol_type = other DNA
tgccaatgtt

SEQ ID NO: 685 - DNA, 10 bp, synthetic construct
  source 1..10: mol_type = other DNA
atgtccgtga

SEQ ID NO: 686 - DNA, 10 bp, synthetic construct
  source 1..10: mol_type = other DNA
ccggtcacta

SEQ ID NO: 687 - DNA, 10 bp, synthetic construct
  source 1..10: mol_type = other DNA
gccagttaat

SEQ ID NO: 688 - DNA, 10 bp, synthetic construct
  source 1..10: mol_type = other DNA
aatacagacg

SEQ ID NO: 689 - DNA, 10 bp, synthetic construct
  source 1..10: mol_type = other DNA
agcaatcaac

SEQ ID NO: 690 - DNA, 10 bp, synthetic construct
  source 1..10: mol_type = other DNA
tatcgcgcgt

SEQ ID NO: 691 - DNA, 10 bp, synthetic construct
  source 1..10: mol_type = other DNA
ccttaattcg

SEQ ID NO: 692 - DNA, 10 bp, synthetic construct
  source 1..10: mol_type = other DNA
gaagttagca

SEQ ID NO: 693 - DNA, 10 bp, synthetic construct
  source 1..10: mol_type = other DNA
ttggcctagg

SEQ ID NO: 694 - DNA, 10 bp, synthetic construct
  source 1..10: mol_type = other DNA
tgttaggtac

SEQ ID NO: 695 - DNA, 10 bp, synthetic construct
  source 1..10: mol_type = other DNA
ttccttgttg

SEQ ID NO: 696 - DNA, 10 bp, synthetic construct
  source 1..10: mol_type = other DNA
actccacgta

SEQ ID NO: 697 - DNA, 10 bp, synthetic construct
  source 1..10: mol_type = other DNA
attcagagtg

SEQ ID NO: 698 - DNA, 10 bp, synthetic construct
  source 1..10: mol_type = other DNA
tcgacattgc

SEQ ID NO: 699 - DNA, 10 bp, synthetic construct
  source 1..10: mol_type = other DNA
ggatgctcgt

SEQ ID NO: 700 - DNA, 10 bp, synthetic construct
  source 1..10: mol_type = other DNA
caactaggaa

SEQ ID NO: 701 - DNA, 10 bp, synthetic construct
  source 1..10: mol_type = other DNA
cctgttcagt

SEQ ID NO: 702 - DNA, 10 bp, synthetic construct
  source 1..10: mol_type = other DNA
aacgtctacc

SEQ ID NO: 703 - DNA, 10 bp, synthetic construct
  source 1..10: mol_type = other DNA
ttgagcatac

SEQ ID NO: 704 - DNA, 10 bp, synthetic construct
  source 1..10: mol_type = other DNA
aactgtgctt

SEQ ID NO: 705 - DNA, 10 bp, synthetic construct
  source 1..10: mol_type = other DNA
attggctgaa

SEQ ID NO: 706 - DNA, 10 bp, synthetic construct
  source 1..10: mol_type = other DNA
gaacctatct

SEQ ID NO: 707 - DNA, 10 bp, synthetic construct
  source 1..10: mol_type = other DNA
tcggcgcata

SEQ ID NO: 708 - DNA, 10 bp, synthetic construct
  source 1..10: mol_type = other DNA
gtcactactg

SEQ ID NO: 709 - DNA, 10 bp, synthetic construct
  source 1..10: mol_type = other DNA
cggtgtgcat

SEQ ID NO: 710 - DNA, 10 bp, synthetic construct
  source 1..10: mol_type = other DNA
agacaattgg

SEQ ID NO: 711 - DNA, 10 bp, synthetic construct
  source 1..10: mol_type = other DNA
tgactgcgaa

SEQ ID NO: 712 - DNA, 10 bp, synthetic construct
  source 1..10: mol_type = other DNA
tcaagccacc

SEQ ID NO: 713 - DNA, 10 bp, synthetic construct
  source 1..10: mol_type = other DNA
taatcagctg

SEQ ID NO: 714 - DNA, 10 bp, synthetic construct
  source 1..10: mol_type = other DNA
gcagattgca

SEQ ID NO: 715 - DNA, 10 bp, synthetic construct
  source 1..10: mol_type = other DNA
atggaactgt

SEQ ID NO: 716 - DNA, 10 bp, synthetic construct
  source 1..10: mol_type = other DNA
cgtacgacga

SEQ ID NO: 717 - DNA, 10 bp, synthetic construct
  source 1..10: mol_type = other DNA
attatcgagg

SEQ ID NO: 718 - DNA, 10 bp, synthetic construct
  source 1..10: mol_type = other DNA
taattcctgc

SEQ ID NO: 719 - DNA, 10 bp, synthetic construct
  source 1..10: mol_type = other DNA
agccaacgac

SEQ ID NO: 720 - DNA, 10 bp, synthetic construct
  source 1..10: mol_type = other DNA
ctaggcggtt

SEQ ID NO: 721 - DNA, 10 bp, synthetic construct
  source 1..10: mol_type = other DNA
tccagacgag

SEQ ID NO: 722 - DNA, 10 bp, synthetic construct
  source 1..10: mol_type = other DNA
cgtctcaacc

SEQ ID NO: 723 - DNA, 10 bp, synthetic construct
  source 1..10: mol_type = other DNA
gtatgtacgt

SEQ ID NO: 724 - DNA, 10 bp, synthetic construct
  source 1..10: mol_type = other DNA
aacacgctaa

SEQ ID NO: 725 - DNA, 10 bp, synthetic construct
  source 1..10: mol_type = other DNA
ggcgatgaag

SEQ ID NO: 726 - DNA, 10 bp, synthetic construct
  source 1..10: mol_type = other DNA
cggatatgta

SEQ ID NO: 727 - DNA, 10 bp, synthetic construct
  source 1..10: mol_type = other DNA
aaggttatgc

SEQ ID NO: 728 - DNA, 10 bp, synthetic construct
  source 1..10: mol_type = other DNA
acatcggtgc

SEQ ID NO: 729 - DNA, 10 bp, synthetic construct
  source 1..10: mol_type = other DNA
ttcagaccgt

SEQ ID NO: 730 - DNA, 10 bp, synthetic construct
  source 1..10: mol_type = other DNA
acagatctcc

SEQ ID NO: 731 - DNA, 10 bp, synthetic construct
  source 1..10: mol_type = other DNA
taactctgag

SEQ ID NO: 732 - DNA, 10 bp, synthetic construct
  source 1..10: mol_type = other DNA
ggtacgaatt

SEQ ID NO: 733 - DNA, 10 bp, synthetic construct
  source 1..10: mol_type = other DNA
atgagctaga

SEQ ID NO: 734 - DNA, 10 bp, synthetic construct
  source 1..10: mol_type = other DNA
cgttaagcat

SEQ ID NO: 735 - DNA, 10 bp, synthetic construct
  source 1..10: mol_type = other DNA
ttgtgttcct

SEQ ID NO: 736 - DNA, 10 bp, synthetic construct
  source 1..10: mol_type = other DNA
actgtgagcg

SEQ ID NO: 737 - DNA, 10 bp, synthetic construct
  source 1..10: mol_type = other DNA
atatgtgtgg

SEQ ID NO: 738 - DNA, 10 bp, synthetic construct
  source 1..10: mol_type = other DNA
gacatgtcat

SEQ ID NO: 739 - DNA, 10 bp, synthetic construct
  source 1..10: mol_type = other DNA
tagccacata

SEQ ID NO: 740 - DNA, 10 bp, synthetic construct
  source 1..10: mol_type = other DNA
cgaggatcac

SEQ ID NO: 741 - DNA, 10 bp, synthetic construct
  source 1..10: mol_type = other DNA
acgcagttag

SEQ ID NO: 742 - DNA, 10 bp, synthetic construct
  source 1..10: mol_type = other DNA
tcacggagct

SEQ ID NO: 743 - DNA, 10 bp, synthetic construct
  source 1..10: mol_type = other DNA
cttcacaacg

SEQ ID NO: 744 - DNA, 10 bp, synthetic construct
  source 1..10: mol_type = other DNA
cgttatgagt

SEQ ID NO: 745 - DNA, 10 bp, synthetic construct
  source 1..10: mol_type = other DNA
agaacagcgt

SEQ ID NO: 746 - DNA, 10 bp, synthetic construct
  source 1..10: mol_type = other DNA
tcctgcaagg

SEQ ID NO: 747 - DNA, 10 bp, synthetic construct
  source 1..10: mol_type = other DNA
cttcagtcaa

SEQ ID NO: 748 - DNA, 10 bp, synthetic construct
  source 1..10: mol_type = other DNA
aataagctcc

SEQ ID NO: 749 - DNA, 10 bp, synthetic construct
  source 1..10: mol_type = other DNA
gaaggcggaa

SEQ ID NO: 750 - DNA, 10 bp, synthetic construct
  source 1..10: mol_type = other DNA
acgattcgaa

SEQ ID NO: 751 - DNA, 10 bp, synthetic construct
  source 1..10: mol_type = other DNA
tcctgctctt

SEQ ID NO: 752 - DNA, 10 bp, synthetic construct
  source 1..10: mol_type = other DNA
ctcgaacacg

SEQ ID NO: 753 - DNA, 10 bp, synthetic construct
  source 1..10: mol_type = other DNA
actgttacac

SEQ ID NO: 754 - DNA, 10 bp, synthetic construct
  source 1..10: mol_type = other DNA
tgacaccaca

SEQ ID NO: 755 - DNA, 10 bp, synthetic construct
  source 1..10: mol_type = other DNA
ggtaaggtcg

SEQ ID NO: 756 - DNA, 10 bp, synthetic construct
  source 1..10: mol_type = other DNA
ctgtcgaggt

SEQ ID NO: 757 - DNA, 10 bp, synthetic construct
  source 1..10: mol_type = other DNA
aagagatagc

SEQ ID NO: 758 - DNA, 10 bp, synthetic construct
  source 1..10: mol_type = other DNA
ccattaccaa

SEQ ID NO: 759 - DNA, 10 bp, synthetic construct
  source 1..10: mol_type = other DNA
cacggacttc

SEQ ID NO: 760 - DNA, 10 bp, synthetic construct
  source 1..10: mol_type = other DNA
gtacattacg

SEQ ID NO: 761 - DNA, 10 bp, synthetic construct
  source 1..10: mol_type = other DNA
taggagacaa

SEQ ID NO: 762 - DNA, 10 bp, synthetic construct
  source 1..10: mol_type = other DNA
agatctatcg

SEQ ID NO: 763 - DNA, 10 bp, synthetic construct
  source 1..10: mol_type = other DNA
ttactgtgcg

SEQ ID NO: 764 - DNA, 10 bp, synthetic construct
  source 1..10: mol_type = other DNA
ccgaatcctc

SEQ ID NO: 765 - DNA, 10 bp, synthetic construct
  source 1..10: mol_type = other DNA
gctggattaa

SEQ ID NO: 766 - DNA, 10 bp, synthetic construct
  source 1..10: mol_type = other DNA
accgtctcgt

SEQ ID NO: 767 - DNA, 10 bp, synthetic construct
  source 1..10: mol_type = other DNA
agagcggaga

SEQ ID NO: 768 - DNA, 10 bp, synthetic construct
  source 1..10: mol_type = other DNA
gaacacggag

SEQ ID NO: 769 - DNA, 10 bp, synthetic construct
  source 1..10: mol_type = other DNA
agtcttgtga

SEQ ID NO: 770 - DNA, 10 bp, synthetic construct
  source 1..10: mol_type = other DNA
ttaaggcgag

SEQ ID NO: 771 - DNA, 10 bp, synthetic construct
  source 1..10: mol_type = other DNA
tcgtcaattc

SEQ ID NO: 772 - DNA, 10 bp, synthetic construct
  source 1..10: mol_type = other DNA
caccacttgt

SEQ ID NO: 773 - DNA, 10 bp, synthetic construct
  source 1..10: mol_type = other DNA
ttcgcagact

SEQ ID NO: 774 - DNA, 10 bp, synthetic construct
  source 1..10: mol_type = other DNA
gagactccgt

SEQ ID NO: 775 - DNA, 10 bp, synthetic construct
  source 1..10: mol_type = other DNA
tgcggagaac

SEQ ID NO: 776 - DNA, 10 bp, synthetic construct
  source 1..10: mol_type = other DNA
acatatcgcg

SEQ ID NO: 777 - DNA, 10 bp, synthetic construct
  source 1..10: mol_type = other DNA
atcaggacag

SEQ ID NO: 778 - DNA, 10 bp, synthetic construct
  source 1..10: mol_type = other DNA
gaattgcgtt

SEQ ID NO: 779 - DNA, 10 bp, synthetic construct
  source 1..10: mol_type = other DNA
tgtggagcct

SEQ ID NO: 780 - DNA, 10 bp, synthetic construct
  source 1..10: mol_type = other DNA
aggaacaaga

SEQ ID NO: 781 - DNA, 10 bp, synthetic construct
  source 1..10: mol_type = other DNA
ccaagcttct

SEQ ID NO: 782 - DNA, 10 bp, synthetic construct
  source 1..10: mol_type = other DNA
tggcattggc

SEQ ID NO: 783 - DNA, 10 bp, synthetic construct
  source 1..10: mol_type = other DNA
acaacgcggt

SEQ ID NO: 784 - DNA, 10 bp, synthetic construct
  source 1..10: mol_type = other DNA
gagctaccac

SEQ ID NO: 785 - DNA, 10 bp, synthetic construct
  source 1..10: mol_type = other DNA
ttgtaaccag

SEQ ID NO: 786 - DNA, 10 bp, synthetic construct
  source 1..10: mol_type = other DNA
gcactattct

SEQ ID NO: 787 - DNA, 10 bp, synthetic construct
  source 1..10: mol_type = other DNA
atggccaaca

SEQ ID NO: 788 - DNA, 10 bp, synthetic construct
  source 1..10: mol_type = other DNA
catactactc

SEQ ID NO: 789 - DNA, 10 bp, synthetic construct
  source 1..10: mol_type = other DNA
agcctgtcca

SEQ ID NO: 790 - DNA, 10 bp, synthetic construct
  source 1..10: mol_type = other DNA
tcatgtcggt

SEQ ID NO: 791 - DNA, 10 bp, synthetic construct
  source 1..10: mol_type = other DNA
acaggtggag

SEQ ID NO: 792 - DNA, 10 bp, synthetic construct
  source 1..10: mol_type = other DNA
caactcaact

SEQ ID NO: 793 - DNA, 10 bp, synthetic construct
  source 1..10: mol_type = other DNA
acttagtagc

SEQ ID NO: 794 - DNA, 10 bp, synthetic construct
  source 1..10: mol_type = other DNA
aggatatcca

SEQ ID NO: 795 - DNA, 10 bp, synthetic construct
  source 1..10: mol_type = other DNA
gcatcaagat

SEQ ID NO: 796 - DNA, 10 bp, synthetic construct
  source 1..10: mol_type = other DNA
aggaatgttc

SEQ ID NO: 797 - DNA, 10 bp, synthetic construct
  source 1..10: mol_type = other DNA
ttctactagc

SEQ ID NO: 798 - DNA, 10 bp, synthetic construct
  source 1..10: mol_type = other DNA
cagaggctat

SEQ ID NO: 799 - DNA, 10 bp, synthetic construct
  source 1..10: mol_type = other DNA
attgtgaagg

SEQ ID NO: 800 - DNA, 10 bp, synthetic construct
  source 1..10: mol_type = other DNA
gtccttaaca

SEQ ID NO: 801 - DNA, 10 bp, synthetic construct
  source 1..10: mol_type = other DNA
aggcgagctt

SEQ ID NO: 802 - DNA, 10 bp, synthetic construct
  source 1..10: mol_type = other DNA
tgctctcgat

SEQ ID NO: 803 - DNA, 10 bp, synthetic construct
  source 1..10: mol_type = other DNA
gaatggttca

SEQ ID NO: 804 - DNA, 10 bp, synthetic construct
  source 1..10: mol_type = other DNA
atcgagaatc

SEQ ID NO: 805 - DNA, 10 bp, synthetic construct
  source 1..10: mol_type = other DNA
ccaacattga

SEQ ID NO: 806 - DNA, 10 bp, synthetic construct
  source 1..10: mol_type = other DNA
attctccagt

SEQ ID NO: 807 - DNA, 10 bp, synthetic construct
  source 1..10: mol_type = other DNA
ggcattatca

SEQ ID NO: 808 - DNA, 10 bp, synthetic construct
  source 1..10: mol_type = other DNA
atcgtacatg

SEQ ID NO: 809 - DNA, 10 bp, synthetic construct
  source 1..10: mol_type = other DNA
tgtctacggc

SEQ ID NO: 810 - DNA, 10 bp, synthetic construct
  source 1..10: mol_type = other DNA
agaaccaatg

SEQ ID NO: 811 - DNA, 10 bp, synthetic construct
  source 1..10: mol_type = other DNA
gtcgacgaca

SEQ ID NO: 812 - DNA, 10 bp, synthetic construct
  source 1..10: mol_type = other DNA
cttggtatgt

SEQ ID NO: 813 - DNA, 10 bp, synthetic construct
  source 1..10: mol_type = other DNA
gacttatcct

SEQ ID NO: 814 - DNA, 10 bp, synthetic construct
  source 1..10: mol_type = other DNA
gcgtgaatca

SEQ ID NO: 815 - DNA, 10 bp, synthetic construct
  source 1..10: mol_type = other DNA
ctcctgttat

SEQ ID NO: 816 - DNA, 10 bp, synthetic construct
  source 1..10: mol_type = other DNA
atattagcgc

SEQ ID NO: 817 - DNA, 33 bp, synthetic construct
  source 1..33: mol_type = other DNA
  modified_base 32^33: mod_base = OTHER; note = thio
acactctttccctacacgacgctcttccgatct

SEQ ID NO: 818 - DNA, 33 bp, synthetic construct
  source 1..33: mol_type = other DNA
  modified_base 1: mod_base = OTHER; note = 5' phosphorylation
gatcggaagagcacacgtctgaactccagtcac

SEQ ID NO: 819 - DNA, 20 bp, synthetic construct
  source 1..20: mol_type = other DNA
acactctttccctacacgac

SEQ ID NO: 820 - DNA, 21 bp, synthetic construct
  source 1..21: mol_type = other DNA
gtgactggagttcagacgtgt

SEQ ID NO: 821 - DNA, 29 bp, synthetic construct
  source 1..29: mol_type = other DNA
  modified_base 1: mod_base = OTHER; note = 5' phosphorylation
aatgatacggcgaccaccgagatctacac

SEQ ID NO: 822 - DNA, 23 bp, synthetic construct
  source 1..23: mol_type = other DNA
  modified_base 1: mod_base = OTHER; note = 5' phosphorylation
aatgatacggcgaccaccgagat

SEQ ID NO: 823 - DNA, 24 bp, synthetic construct
  source 1..24: mol_type = other DNA
caagcagaagacggcatacgagat

SEQ ID NO: 824 - DNA, 24 bp, synthetic construct
  source 1..24: mol_type = other DNA
atctcgtatgccgtcttctgcttg

SEQ ID NO: 825 - DNA, 21 bp, synthetic construct
  source 1..21: mol_type = other DNA
caagcagaagacggcatacga